SEQUENCE LISTING (45 sequences)

SEQ ID NO: 1 - protein, 737 aa, Homo sapiens
MNGDSRAAVVTSPPPTTAPHKERYFDRVDENNPEYLRERNMAPDLRQDFNMMEQKKRVSMILQSPAFCEELESMIQEQFKKGKNPTGLLALQQIADFMTTNVPNVYPAAPQGGMAALNMSLGMVTPVNDLRGSDSIAYDKGEKLLRCKLAAFYRLADLFGWSQLIYNHITTRVNSEQEHFLIVPFGLLYSEVTASSLVKINLQGDIVDRGSTNLGVNQAGFTLHSAIYAARPDVKCVVHIHTPAGAAVSAMKCGLLPISPEALSLGEVAYHDYHGILVDEEEKVLIQKNLGPKSKVLILRNHGLVSVGESVEEAFYYIHNLVVACEIQVRTLASAGGPDNLVLLNPEKYKAKSRSPGSPVGEGTGSPPKWQIGEQEFEALMRMLDNLGYRTGYPYRYPALREKSKKYSDVEVPASVTGYSFASDGDSGTCSPLRHSFQKQQREKTRWLNSGRGDEASEEGQNGSSPKSKTKWTKEDGHRTSTSAVPNLFVPLNTNPKEVQEMRNKIREQNLQDIKTAGPQSQVLCGVVMDRSLVQGELVTASKAIIEKEYQPHVIVSTTGPNPFTTLTDRELEEYRREVERKQKGSEENLDEAREQKEKSPPDQPAVPHPPPSTPIKLEEDLVPEPTTGDDSDAATFKPTLPDLSPDEPSEALGFPMLEKEEEAHRPPSPTEAPTEASPEPAPDPAPVAEEAAPSAVEEGAAADPGSDGSPGKSPSKKKKKFRTPSFLKKSKKKSDS

SEQ ID NO: 2 - protein, 191 aa, Homo sapiens
MVKKLVMAQKRGETRALCLGVTMVVCAVITYYILVTTVLPLYQKSVWTQESKCHLIETNIRDQEELKGKKVPQYPCLWVNVSAAGRWAVLYHTEDTRDQNQQCSYIPGSVDNYQTARADVEKVRAKFQEQQVFYCFSAPRGNETSVLFQRLYGPQALLFSLFWPTFLLTGGLLIIAMVKSNQYLSILAAQK

SEQ ID NO: 3 - protein, 2221 aa, Homo sapiens
MVNENTRMYIPEENHQGSNYGSPRPAHANMNANAAAGLAPEHIPTPGAALSWQAAIDAARQAKLMGSAGNATISTVSSTQRKRQQYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIADVPAEDDPSPCALETGHGRQCQNGTVCKPGWDGPKHGITNFDNFAFAMLTVFQCITMEGWTDVLYWVNDAVGRDWPWIYFVTLIIIGSFFVLNLVLGVLSGEFSKEREKAKARGDFQKLREKQQLEEDLKGYLDWITQAEDIDPENEDEGMDEEKPRNMSMPTSETESVNTENVAGGDIEGENCGARLAHRISKSKFSRYWRRWNRFCRRKCRAAVKSNVFYWLVIFLVFLNTLTIASEHYNQPNWLTEVQDTANKALLALFTAEMLLKMYSLGLQAYFVSLFNRFDCFVVCGGILETILVETKIMSPLGISVLRCVRLLRIFKITRYWNSLSNLVASLLNSVRSIASLLLLLFLFIIIFSLLGMQLFGGKFNFDEMQTRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKKQELVEKPAVGESKEEKIELKSITADGESPPATKINMDDLQPNENEDKSPYPNPETTGEEDEEEPEMPVGPRPRPLSELHLKEKAVPMPEASAFFIFSSNNRFRLQCHRIVNDTIFTNLILFFILLSSISLAAEDPVQHTSFRNHILFYFDIVFTTIFTIEIALKILGNADYVFTSIFTLEIILKMTAYGAFLHKGSFCRNYFNILDLLVVSVSLISFGIQSSAINVVKILRVLRVLRPLRAINRAKGLKHVVQCVFVAIRTIGNIVIVTTLLQFMFACIGVQLFKGKLYTCSDSSKQTEAECKGNYITYKDGEVDHPIIQPRSWENSKFDFDNVLAAMMALFTVSTFEGWPELLYRSIDSHTEDKGPIYNYRVEISIFFIIYIIIIAFFMMNIFVGFVIVTFQEQGEQEYKNCELDKNQRQCVEYALKARPLRRYIPKNQHQYKVWYVVNSTYFEYLMFVLILLNTICLAMQHYGQSCLFKIAMNILNMLFTGLFTVEMILKLIAFKPKGYFSDPWNVFDFLIVIGSIIDVILSETNHYFCDAWNTFDALIVVGSIVDIAITEVNPAEHTQCSPSMNAEENSRISITFFRLFRVMRLVKLLSRGEGIRTLLWTFIKSFQALPYVALLIVMLFFIYAVIGMQVFGKIALNDTTEINRNNNFQTFPQAVLLLFRCATGEAWQDIMLACMPGKKCAPESEPSNSTEGETPCGSSFAVFYFISFYMLCAFLIINLFVAVIMDNFDYLTRDWSILGPHHLDEFKRIWAEYDPEAKGRIKHLDVVTLLRRIQPPLGFGKLCPHRVACKRLVSMNMPLNSDGTVMFNATLFALVRTALRIKTEGNLEQANEELRAIIKKIWKRTSMKLLDQVVPPAGDDEVTVGKFYATFLIQEYFRKFKKRKEQGLVGKPSQRNALSLQAGLRTLHDIGPEIRRAISGDLTAEEELDKAMKEAVSAASEDDIFRRAGGLFGNHVSYYQSDGRSAFPQTFTTQRPLHINKAGSSQGDTESPSHEKLVDSTFTPSSYSSTGSNANINNANNTALGRLPRPAGYPSTVSTVEGHGPPLSPAIRVQEVAWKLSSNRERHVPVCEDLELRRDSGSAGTQAHCLLLRRANPSRCHSRESQAAMAGQEETSQDETYEVKMNHDTEACSEPSLLSTEMLSYQDDENRQLTLPEEDKRDIRQSPKRGFLRSASLGRRASFHLECLKRQKDRGGDISQKTVLPLHLVHHQALAVAGLSPLLQRSHSPASFPRPFATPPATPGSRGWPPQPVPTLRLEGVESSEKLNSSFPSIHCGSWAETTPGGGGSSAARRVRPVSLMVPSQAGAPGRQFHGSASSLVEAVLISEGLGQFAQDPKFIEVTTQELADACDMTIEEMESAADNILSGGAPQSPNGALLPFVNCRDAGQDRAGGEEDAGCVRARGAPSEEELQDSRVYVSSL

SEQ ID NO: 4 - protein, 605 aa, Homo sapiens
MQCCGLVHRRRVRVSYGSADSYTSRPSDSDVSLEEDREAVRREAERQAQAQLEKAKTKPVAFAVRTNVSYSAAHEDDVPVPGMAISFEAKDFLHVKEKFNNDWWIGRLVKEGCEIGFIPSPVKLENMRLQHEQRAKQGKFYSSKSGGNSSSSLGDIVPSSRKSTPPSSAIDIDATGLDAEENDIPANHRSPKPSANSVTSPHSKEKRMPFFKKTEHTPPYDVVPSMRPVVLVGPSLKGYEVTDMMQKALFDFLKHRFEGRISITRVTADISLAKRSVLNNPSKHAIIERSNTRSSLAEVQSEIERIFELARTLQLVVLDADTINHPAQLSKTSLAPIIVYVKISSPKVLQRLIKSRGKSQAKHLNVQMVAADKLAQCPPELFDVILDENQLEDACEHLADYLEAYWKATHPPSSSLPNPLLSRTLATSSLPLSPTLASNSQGSQGDQRTDRSAPIRSASQAEEEPSVEPVKKSQHRSSSSAPHHNHRSGTSRGLSRQETFDSETQESRDSAYVEPKEDYSHDHVDHYASHRDHNHRDETHGSSDHRHRESRHRSRDVDREQDHNECNKQRSRHKSKDRYCEKDGEVISKKRNEAGEWNRDVYIRQ

SEQ ID NO: 5 - protein, 674 aa, Homo sapiens
MPSYTVTVATGSQWFAGTDDYIYLSLVGSAGCSEKHLLDKPFYNDFERGAVDSYDVTVDEELGEIQLVRIEKRKYWLNDDWYLKYITLKTPHGDYIEFPCYRWITGDVEVVLRDGRAKLARDDQIHILKQHRRKELETRQKQYRWMEWNPGFPLSIDAKCHKDLPRDIQFDSEKGVDFVLNYSKAMENLFINRFMHMFQSSWNDFADFEKIFVKISNTISERVMNHWQEDLMFGYQFLNGCNPVLIRRCTELPEKLPVTTEMVECSLERQLSLEQEVQQGNIFIVDFELLDGIDANKTDPCTLQFLAAPICLLYKNLANKIVPIAIQLNQIPGDENPIFLPSDAKYDWLLAKIWVRSSDFHVHQTITHLLRTHLVSEVFGIAMYRQLPAVHPIFKLLVAHVRFTIAINTKAREQLICECGLFDKANATGGGGHVQMVQRAMKDLTYASLCFPEAIKARGMESKEDIPYYFYRDDGLLVWEAIRTFTAEVVDIYYEGDQVVEEDPELQDFVNDVYVYGMRGRKSSGFPKSVKSREQLSEYLTVVIFTASAQHAAVNFGQYDWCSWIPNAPPTMRAPPPTAKGVVTIEQIVDTLPDRGRSCWHLGAVWALSQFQENELFLGMYPEEHFIEKPVKEAMARFRKNLEAIVSVIAERNKKKQLPYYYLSPDRIPNSVAI

SEQ ID NO: 6 - protein, 477 aa, Homo sapiens
MGAGVLVLGASEPGNLSSAAPLPDGAATAARLLVPASPPASLLPPASESPEPLSQQWTAGMGLLMALIVLLIVAGNVLVIVAIAKTPRLQTLTNLFIMSLASADLVMGLLVVPFGATIVVWGRWEYGSFFCELWTSVDVLCVTASIETLCVIALDRYLAITSPFRYQSLLTRARARGLVCTVWAISALVSFLPILMHWWRAESDEARRCYNDPKCCDFVTNRAYAIASSVVSFYVPLCIMAFVYLRVFREAQKQVKKIDSCERRFLGGPARPPSPSPSPVPAPAPPPGPPRPAAAAATAPLANGRAGKRRPSRLVALREQKALKTLGIIMGVFTLCWLPFFLANVVKAFHRELVPDRLFVFFNWLGYANSAFNPIIYCRSPDFRKAFQGLLCCARRAARRRHATHGDRPRASGCLARPGPPPSPGAASDDDDDDVVGATPPARLLEPWAGCNGGAAADSDSSLDEPCRPGFASESKV

SEQ ID NO: 7 - protein, 413 aa, Homo sapiens [sequence omitted]

SEQ ID NO: 8 - protein, 161 aa, Homo sapiens
MDQETVGNVVLLAIVTLISVVQNGFFAHKVEHESRTQNGRSFQRTGTLAFERVYTANQNCVDAYPTFLAVLWSAGLLCSQVPAAFAGLMYLFVRQKYFVGYLGERTQSTPGYIFGKRIILFLFLMSVAGIFNYYLIFFFGSDFENYIKTISTTISPLLLIP

SEQ ID NO: 9 - protein, 611 aa, Homo sapiens
MPEIVDTCSLASPASVCRTKHLHLRCSVDFTRRTLTGTAALTVQSQEDNLRSLVLDTKDLTIEKVVINGQEVKYALGERQSYKGSPMEISLPIALSKNQEIVIEISFETSPKSSALQWLTPEQTSGKEHPYLFSQCQAIHCRAILPCQDTPSVKLTYTAEVSVPKELVALMSAIRDGETPDPEDPSRKIYKFIQKVPIPCYLIALVVGALESRQIGPRTLVWSEKEQVEKSAYEFSETESMLKIAEDLGGPYVWGQYDLLVLPPSFPYGGMENPCLTFVTPTLLAGDKSLSNVIAHEISHSWTGNLVTNKTWDHFWLNEGHTVYLERHICGRLFGEKFRHFNALGGWGELQNSVKTFGETHPFTKLVVDLTDIDPDVAYSSVPYEKGFALLFYLEQLLGGPEIFLGFLKAYVEKFSYKSITTDDWKDFLYSYFKDKVDVLNQVDWNAWLYSPGLPPIKPNYDMTLTNACIALSQRWITAKEDDLNSFNATDLKDLSSHQLNEFLAQTLQRAPLPLGHIKRMQEVYNFNAINNSEIRFRWLRLCIQSKWEDAIPLALKMATEQGRMKFTRPLFKDLAAFDKSHDQAVRTYQEHKASMHPVTAMLVGKDLKVD

SEQ ID NO: 10 - DNA, 23 bp, Artificial Sequence
  Description of Artificial Sequence Synthetic Primer
aaatacagcgatgtggaggttcc

SEQ ID NO: 11 - DNA, 23 bp, Artificial Sequence
  Description of Artificial Sequence Synthetic Primer
cagttggtaatacagcttggcac

SEQ ID NO: 12 - DNA, 15 bp, Artificial Sequence
  Description of Artificial Sequence Synthetic Primer
tgcttccattctgcc

SEQ ID NO: 13 - DNA, 20 bp, Artificial Sequence
  Description of Artificial Sequence Synthetic Primer
gattggactggaagagtggg

SEQ ID NO: 14 - DNA, 19 bp, Artificial Sequence
  Description of Artificial Sequence Synthetic Primer
tgctcctacatcccaggca

SEQ ID NO: 15 - DNA, 21 bp, Artificial Sequence
  Description of Artificial Sequence Synthetic Primer
aatttggctctgaccttctcc

SEQ ID NO: 16 - DNA, 19 bp, Artificial Sequence
  Description of Artificial Sequence Synthetic Primer
gccgtctggtaattgtcca

SEQ ID NO: 17 - DNA, 21 bp, Artificial Sequence
  Description of Artificial Sequence Synthetic Primer
ggatcgctactttgccattac

SEQ ID NO: 18 - DNA, 19 bp, Artificial Sequence
  Description of Artificial Sequence Synthetic Primer
ggcataggcttggttcgtg

SEQ ID NO: 19 - DNA, 17 bp, Artificial Sequence
  Description of Artificial Sequence Synthetic Primer
cattcagatgcactggt

SEQ ID NO: 20 - DNA, 11 bp, Homo sapiens
agcacrgtcag

SEQ ID NO: 21 - DNA, 11 bp, Homo sapiens
cgttcygrtgt

SEQ ID NO: 22 - DNA, 300 bp, Homo sapiens
gacacctcgctgaggagagacccaggagcgaggcccctgccccgcccgaggcgaggtcccgcccagtcggcgccgcgtgaagagtgggagagaagtactgcgggggcgggggcgggggcgggggcgggggcgggggcagccgggagcctggagccagaccggggcggggccgggaccggggccagggaccagtggtgggaggaggctgcggcgctagatgcggacacctggaccgccgcgccgaggctcccggcgctcgctgctcccgcggcccgcgccatgccctcctacacggtcacc

SEQ ID NO: 23 - DNA, 52 bp, Homo sapiens
tcttgcccattcagatgcactggtacmgggccacccaccaggaagccatcaa

SEQ ID NO: 24 - DNA, 52 bp, Homo sapiens
gggagggcaggtggagaaggcattgtkctgcaagtggggagcagccctgggg

SEQ ID NO: 25 - DNA, 52 bp, Homo sapiens
tgggagggcaggtggagaaggcattgkgctgcaagtggggagcagccctggg

SEQ ID NO: 26 - DNA, 52 bp, Homo sapiens
gataagtgctttaaatgcattcttgtrtctaatccttacagtaaccctgctg

SEQ ID NO: 27 - DNA, 52 bp, Homo sapiens
tggtataacactgtgttgatccacacygttcactgttctatgtaaggtagtt

SEQ ID NO: 28 - DNA, 52 bp, Homo sapiens
cctcgccccgccggctaccccagcacrgtcagcactgtggagggccacgggc

SEQ ID NO: 29 - DNA, 52 bp, Homo sapiens
ctgtcccagcagggaaaggcacgttcyggtgtgtgaggatctggagctcagg

SEQ ID NO: 30 - DNA, 52 bp, Homo sapiens
cctcgttgctgcctcccgccagcgaargccccgagccgctgtctcagcagtg

SEQ ID NO: 31 - DNA, 52 bp, Homo sapiens
gccccgacttccgcaaggccttccagsgactgctctgctgcgcgcgcagggc

SEQ ID NO: 32 - DNA, 52 bp, Homo sapiens; identical to SEQ ID NO: 26

SEQ ID NO: 33 - DNA, 52 bp, Homo sapiens; identical to SEQ ID NO: 27

SEQ ID NO: 34 - DNA, 52 bp, Homo sapiens
acttcatagtgtctaccactggccccrcggggctctgcagcttccacttgag

SEQ ID NO: 35 - DNA, 52 bp, Homo sapiens; identical to SEQ ID NO: 28

SEQ ID NO: 36 - DNA, 52 bp, Homo sapiens; identical to SEQ ID NO: 29

SEQ ID NO: 37 - DNA, 3970 bp, Homo sapiens
gcacccaggtcgggcggtgggggcgagcggaggggctgaggggcggagaggcctggcgggccgctgctgcgggccaggggacgggggcggagccggagccggagccgacgggcggtggccgcactgggaccccggaatcccgcgcgctgcccacgattcgcttctgaggaacctagaaagattgtacaatgaatggtgattctcgtgctgcggtggtgacctcaccacccccgaccacagcccctcacaaggagaggtacttcgaccgagtagatgagaacaacccagagtacttgagggagaggaacatggcaccagaccttcgccaggacttcaacatgatggagcaaaagaagagggtgtccatgattctgcaaagccctgctttctgtgaagaattggaatcaatgatacaggagcaatttaagaaggggaagaaccccacaggcctattggcattacagcagattgcagattttatgaccacgaatgtaccaaatgtctacccagcagctccgcaaggagggatggctgccttaaacatgagtcttggtatggtgactcctgtgaacgatcttagaggatctgattctattgcgtatgacaaaggagagaagttattacggtgtaaattggcagcgttttatagactagcagatctctttgggtggtctcagcttatctacaatcatatcacaaccagagtgaactccgagcaggaacacttcctcattgtcccttttgggcttctttacagtgaagtgactgcatccagtttggttaagatcaatctacaaggagatatagtagatcgtggaagcactaatctgggagtgaatcaggccggcttcaccttacactctgcaatttatgctgcacgcccggacgtgaagtgcgtcgtgcacattcacaccccagcaggggctgcggtctctgcaatgaaatgtggcctcttgccaatctccccggaggcgctttcccttggagaagtggcttatcatgactaccatggcattctggttgatgaagaggaaaaagttttgattcagaaaaatctggggcctaaaagcaaggttcttattctccggaaccatgggctcgtgtcagttggagagagcgttgaggaggccttctattacatccataaccttgtggttgcctgtgagatccaggttcgaactctggccagtgcaggaggaccagacaacttagtcctgctgaatcctgagaagtacaaagccaagtcccgttccccagggtctccggtaggggaaggcactggatcgcctcccaagtggcagattggtgagcaggaatttgaagccctcatgcggatgctcgataatctgggctacagaactggctacccttatcgataccctgctctgagagagaagtctaaaaaatacagcgatgtggaggttcctgctagtgtcacaggttactcctttgctagtgacggtgattcgggcacttgctccccactcagacacagttttcagaagcagcagcgggagaagacaagatggctgaactctggccggggcgacgaagcttccgaggaagggcagaatggaagcagtcccaagtcgaagactaagtggactaaagaggatggacatagaacttccacctctgctgtccctaacctgtttgttccattgaacactaacccaaaagaggtccaggagatgaggaacaagatccgagagcagaatttacaggacattaagacggctggccctcagtcccaggttttgtgtggtgtagtgatggacaggagcctcgtccagggagagctggtgacggcctccaaggccatcattgaaaaggagtaccagccccacgtcattgtgagcaccacgggccccaaccccttcaccacactcacagaccgtgagctggaggagtaccgcagggaggtggagaggaagcagaagggctctgaagagaatctggacgaggctagagaacagaaagaaaagagtcctccagaccagcctgcggtcccccacccgcctcccagcactcccatcaagctggaggaagaccttgtgccggagccgactactggagatgacagtgatgctgccacctttaagccaactctccccgatctgtcccctgatgaaccttcagaagcactcggcttcccaatgttagagaaggaggaggaagcccatagacccccaagccccactgaggcccctactgaggccagccccgagccagccccagacccagccccggtggctgaagaggctgccccctcagctgtcgaggagggggccgccgcggaccctggcagcgatgggtctccaggcaagtccccgtccaaaaagaagaagaagttccgtaccccgtcctttctgaagaagagcaagaagaagagtgactcctgaaagccctgcgctaacactgtcctgtccggagcgaccctggctctgccagcgtccccggccacgtctgtgctctgtccttgtgtaatggaatgcaaaaaagccaagccctccgcctagaggtcccctcacgtgaccagccccgtgtagccccgggctgacccagtgtgtgctcagcagccccaccccaccctgccccttgtcctctcagagcctcagcttctgggggagacatgctctccccacaggggggaggcactaagtcatggtcctggctggaaggtactgaaggcttctgcagctttggctgcacgtcaccctcctgagcctcacctttcctgccgtccctcctgttgtgaaatcaccacattctgtctctgcttggcttcccctccaccctaaagtctcaggtgacggactcagactcctggcttcatgtggcattctctctgctcagtgatctcacttaaatctatatacaaagccttggtcccgtgaaaacactcgtgtgcccaccagcggccttgaagaggcaggtctgggccagatgctgggcaggaaaccccagcggcagatgggcctgtgtgcacccaacgtgatgctatgcatgtctgaccgacgatccctcgaccagaatcagattcaggagctcagtttctttttcacttgggtctctggattcctgtcatagggaaggtatatcaggaggggaagaggcctttctagaattttctttgagcaggtttacaatttagcttacatttttcgactgtgaacgtgaataggctgctttttgctttcttctttccagaccccacagtagagcacttttcacttatttgggggaggcttcaggggactgttctcaccttaactcagccagaaagatgccctagttgtgatcaaaggtaactcgaggtggagggtagccctggggcccctcgacatcaccgtcattgatggagcctgaaccgtgtgctcctcggcagatgctgttgttgttacttccctccaagaggctggaaaagggctcagagctgctgagcaggaaccggagggtgacccatttcaggaggtgccggtaccagcctgactaggtacaggcaagcttgtgtgggcccaacaggcccttggtagagctggtgccagatgtgggctcagatcctgggcatgatgggccgagccacctcggatcccactgattggccagccgagcgagaaccaggctgctgcatggcactgaccgccgcttccagcttcctctgagccgcagggcctgctacgcgggcaagcgtgctgcctctcttctgtgtcgttttgttgccaaggcagaatgaaaagtccttaaccgtggactcttcctttatcccctcctttaccccacatatgcaatgacttttaattttcacttttgtagtttaatcctttgtattacaacatgaaatatagttgcatatatggacaccgacttgggaggacaggtcctgaatgtcctttctccagtgtaacatgttttactcacaaataaaattctttcagcaaaaaaaaaaaaaaaaaaaaaaaaaaaaaaa

SEQ ID NO: 38 - DNA, 1518 bp, Homo sapiens
cagagagaagaagaaatatttccaaatataccaggctgatctttcgtagtttctcagtcttgctgtctgttgttgtttgaaatctgtttcccacgagactctgggaagatggaggtgcagggcttgggtaacagctgccttcctcagggctgggtacctgctctccgagtctgtgtctccaggctgtgtgctcagacttgaccatgtttgggcatggaggcttgccccagatggggaccaaaagctgcttccctggagacgggcggggaggccaaacttcctgctgaagctctgtggcctcttttggggtgggggcgggggtccaggcagaaagaaactgtctgctgctcaagacccacaggacgccgggaagactaaatgatcactgcccccagtgaatatggtgaagaagctggtgatggcccagaagcggggagagacacgagccctttgcctgggtgtaaccatggtggtgtgtgccgtcatcacctactacatcctggtcacgactgtgctgcccctctaccagaaaagcgtgtggacccaggaatccaagtgccacctgattgagaccaacatcagggaccaggaggagctgaagggcaagaaggtgccccagtacccatgcctgtgggtcaacgtgtcagctgccggcaggtgggctgtgctgtaccacacggaggacactcgggaccagaaccagcagtgctcctacatcccaggcagcgtggacaattaccagacggcccgggccgacgtggagaaggtcagagccaaattccaagagcagcaggtcttctactgcttctccgcacctcgggggaacgaaaccagcgtcctattccagcgcctctacgggccccaggccctcctcttctccctcttctggcccaccttcctgctgaccggtggcctcctcattatcgccatggtgaagagcaaccagtacctgtccatcctggcggcccagaagtagagccatccatccatgccataccacttgtcagggcacaggggactggctgggcccccagggctgctccccacttgcagcacaatgccttctccacctgccctcccactcttccagtccaatccacgctgtcttctgttgcaggactaacctttgagaaatccttttgtgaagtcattgcctgctcaagaatgtacagtggctccccaatgccttggaggccataaggccagccagttctagctctctattacctgtccccactcaactgactcatacctgtttccggctgcatcactatgtgccccacagagaacgatgatcgtcacctctgtgcctgagttctccctgttgtctcaaagcggtacccatcctcccccagaagctgtccccagcgagcctcccttctttgtttgaattctctaataagagcaacagcagctcccatttctagaacacatttacagtattactattttctaggatataaagtgccatatatatttttaattccaatattaataaatgtatgccaaacaacaata

SEQ ID NO: 39 - DNA, 2862 bp, Homo sapiens
gcaccacgccgcccgggcttctggggtgttccccaaccacggcccagccctgccacaccccccgcccccggcctccgcagctcggcatgggcgcgggggtgctcgtcctgggcgcctccgagcccggtaacctgtcgtcggccgcaccgctccccgacggcgcggccaccgcggcgcggctgctggtgcccgcgtcgccgcccgcctcgttgctgcctcccgccagcgaaagccccgagccgctgtctcagcagtggacagcgggcatgggtctgctgatggcgctcatcgtgctgctcatcgtggcgggcaatgtgctggtgatcgtggccatcgccaagacgccgcggctgcagacgctcaccaacctcttcatcatgtccctggccagcgccgacctggtcatggggctgctggtggtgccgttcggggccaccatcgtggtgtggggccgctgggagtacggctccttcttctgcgagctgtggacctcagtggacgtgctgtgcgtgacggccagcatcgagaccctgtgtgtcattgccctggaccgctacctcgccatcacctcgcccttccgctaccagagcctgctgacgcgcgcgcgggcgcggggcctcgtgtgcaccgtgtgggccatctcggccctggtgtccttcctgcccatcctcatgcactggtggcgggcggagagcgacgaggcgcgccgctgctacaacgaccccaagtgctgcgacttcgtcaccaaccgggcctacgccatcgcctcgtccgtagtctccttctacgtgcccctgtgcatcatggccttcgtgtacctgcgggtgttccgcgaggcccagaagcaggtgaagaagatcgacagctgcgagcgccgtttcctcggcggcccagcgcggccgccctcgccctcgccctcgcccgtccccgcgcccgcgccgccgcccggacccccgcgccccgccgccgccgccgccaccgccccgctggccaacgggcgtgcgggtaagcggcggccctcgcgcctcgtggccctgcgcgagcagaaggcgctcaagacgctgggcatcatcatgggcgtcttcacgctctgctggctgcccttcttcctggccaacgtggtgaaggccttccaccgcgagctggtgcccgaccgcctcttcgtcttcttcaactggctgggctacgccaactcggccttcaaccccatcatctactgccgcagccccgacttccgcaaggccttccagggactgctctgctgcgcgcgcagggctgcccgccggcgccacgcgacccacggagaccggccgcgcgcctcgggctgtctggcccggcccggacccccgccatcgcccggggccgcctcggacgacgacgacgacgatgtcgtcggggccacgccgcccgcgcgcctgctggagccctgggccggctgcaacggcggggcggcggcggacagcgactcgagcctggacgagccgtgccgccccggcttcgcctcggaatccaaggtgtagggcccggcgcggggcgcggactccgggcacggcttcccaggggaacgaggagatctgtgtttacttaagaccgatagcaggtgaactcgaagcccacaatcctcgtctgaatcatccgaggcaaagagaaaagccacggaccgttgcacaaaaaggaaagtttgggaagggatgggagagtggcttgctgatgttccttgttgttttttttttcttttcttttctttcttcttcttttttttttttttttttttttctgtttgtggtccggccttcttttgtgtgtgcgtgtgatgcatctttagatttttttcccccaccaggtggtttttgacactctctgagaggaccggagtggaagatgggtgggttaggggaagggagaagcattaggaggggattaaaatcgatcatcgtggctcccatccctttcccgggaacaggaacacactaccagccagagagaggagaatgacagtttgtcaagacatatttccttttgctttccagagaaatttcattttaatttctaagtaatgatttctgctgttatgaaagcaaagagaaaggatggaggcaaaataaaaaaaaatcacgtttcaagaaatgttaagctcttcttggaacaagccccaccttgctttccttgtgtagggcaaacccgctgtcccccgcgcgcctgggtggtcaggctgagggatttctacctcacactgtgcatttgcacagcagatagaaagacttgtttatattaaacagcttatttatgtatcaatattagttggaaggaccaggcgcagagcctctctctgtgacatgtgactctgtcaattgaagacaggacattaaaagagagcgagagagagaaacagttcagattactgcacatgtggataaaaacaaaaacaaaaaaaaggagtggttcaaaatgccatttttgcacagtgttaggaattacaaaatccacagaagatgttacttgcacaaaaagaaattaaatattttttaaagggagaggggctgggcagatcttaaataaaattcaaactctacttctgttgtctagtatgttattgagctaatgattcattgggaaaatacctttttatactcctttatcatggtactgtaactgtatccatattataaatataattatcttaaggattttttatttttttttatgtccaagtgcccacgtgaatttgctggtgaaagttagcacttgtgtgtaaattctacttcctcttgtgtgttttaccaagtatttatactctggtgcaactaactactgtgtgaggaattggtccatgtgcaataaataccaatgaagcacaatcaa

SEQ ID NO: 40 - DNA, 1242 bp, Homo sapiens
atggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttatctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctgtaa

SEQ ID NO: 41 - DNA, 2568 bp, Homo sapiens
gccagggaccagtggtgggaggaggctgcggcgctagatgcggacacctggaccgccgcgccgaggctcccggcgctcgctgctcccgcggcccgcgccatgccctcctacacggtcaccgtggccactggcagccagtggttcgccggcactgacgactacatctacctcagcctcgtgggctcggcgggctgcagcgagaagcacctgctggacaagcccttctacaacgacttcgagcgtggcgcggtggattcatacgacgtgactgtggacgaggaactgggcgagatccagctggtcagaatcgagaagcgcaagtactggctgaatgacgactggtacctgaagtacatcacgctgaagacgccccacggggactacatcgagttcccctgctaccgctggatcaccggcgatgtcgaggttgtcctgagggatggacgcgcaaagttggcccgagatgaccaaattcacattctcaagcaacaccgacgtaaagaactggaaacacggcaaaaacaatatcgatggatggagtggaaccctggcttccccttgagcatcgatgccaaatgccacaaggatttaccccgtgatatccagtttgatagtgaaaaaggagtggactttgttctgaattactccaaagcgatggagaacctgttcatcaaccgcttcatgcacatgttccagtcttcttggaatgacttcgccgactttgagaaaatctttgtcaagatcagcaacactatttctgagcgggtcatgaatcactggcaggaagacctgatgtttggctaccagttcctgaatggctgcaaccctgtgttgatccggcgctgcacagagctgcccgagaagctcccggtgaccacggagatggtagagtgcagcctggagcggcagctcagcttggagcaggaggtccagcaagggaacattttcatcgtggactttgagctgctggatggcatcgatgccaacaaaacagacccctgcacactccagttcctggccgctcccatctgcttgctgtataagaacctggccaacaagattgtccccattgccatccagctcaaccaaatcccgggagatgagaaccctattttcctcccttcggatgcaaaatacgactggcttttggccaaaatctgggtgcgttccagtgacttccacgtccaccagaccatcacccaccttctgcgaacacatctggtgtctgaggtttttggcattgcaatgtaccgccagctgcctgctgtgcaccccattttcaagctgctggtggcacacgtgagattcaccattgcaatcaacaccaaggcccgtgagcagctcatctgcgagtgtggcctctttgacaaggccaacgccacagggggcggtgggcacgtgcagatggtgcagagggccatgaaggacctgacctatgcctccctgtgctttcccgaggccatcaaggcccggggcatggagagcaaagaagacatcccctactacttctaccgggacgacgggctcctggtgtgggaagccatcaggacgttcacggccgaggtggtagacatctactacgagggcgaccaggtggtggaggaggacccggagctgcaggacttcgtgaacgatgtctacgtgtacggcatgcggggccgcaagtcctcaggcttccccaagtcggtcaagagccgggagcagctgtcggagtacctgaccgtggtgatcttcaccgcctccgcccagcacgccgcggtcaacttcggccagtacgactggtgctcctggatccccaatgcgcccccaaccatgcgagccccgccaccgactgccaagggcgtggtgaccattgagcagatcgtggacacgctgcccgaccgcggccgctcctgctggcatctgggtgcagtgtgggcgctgagccagttccaggaaaacgagctgttcctgggcatgtacccagaagagcattttatcgagaagcctgtgaaggaagccatggcccgattccgcaagaacctcgaggccattgtcagcgtgattgctgagcgcaacaagaagaagcagctgccatattactacttgtccccagaccggattccgaacagtgtggccatctgagcacactgccagtctcactgtgggaaggccagctgccccagccagatggactccagcctgcctggcaggctgtctggccaggcctcttggcagtcacatctcttcctccgaggccagtacctttccatttattctttgatcttcagggaactgcatagattgatcaaagtgtaaacaccatagggacccattctacacagagcaggactgcacagcgtcctgtccacacccagctcagcatttccacaccaagcagcaacagcaaatcacgaccactgatagatgtctattcttgttggagacatgggatgattattttctgttctatttgtgcttagtccaattccttgcacatagtaggtacccaattcaattactattgaatgaattaagaattggttgccataaaaataaatcagttcatttaaaaaaaaaaaaaaaaaa

SEQ ID NO: 42 - DNA, 2060 bp, Homo sapiens
ctctatcgacgagtctggtagctgagcgttgggctgtaggtcgctgtgctgtgtgatcccccagagccatgcccgagatagtggatacctgttcgttggcctctccggcttccgtctgccggaccaagcacctgcacctgcgctgcagcgtcgactttactcgccggacgctgaccgggactgctgctctcacggtccagtctcaggaggacaatctgcgcagcctggttttggatacaaaggaccttacaatagaaaaagtagtgatcaatggacaagaagtcaaatatgctcttggagaaagacaaagttacaagggatcgccaatggaaatctctcttcctatcgctttgagcaaaaatcaagaaattgttatagaaatttcttttgagacctctccaaaatcttctgctctccagtggctcactcctgaacagacttctgggaaggaacacccatatctctttagtcagtgccaggccatccactgcagagcaatccttccttgtcaggacactccttctgtgaaattaacctatactgcagaggtgtctgtccctaaagaactggtggcacttatgagtgctattcgtgatggagaaacacctgacccagaagacccaagcaggaaaatatacaaattcatccaaaaagttccaataccctgctacctgattgctttagttgttggagctttagaaagcaggcaaattggcccaagaactttggtgtggtctgagaaagagcaggtggaaaagtctgcttatgagttttctgagactgaatctatgcttaaaatagcagaagatctgggaggaccgtatgtatggggacagtatgacctattggtcctgccaccatccttcccttatggtggcatggagaatccttgccttacttttgtaactcctactctactggcaggcgacaagtcactctccaatgtcattgcacatgaaatatctcatagctggacagggaatctagtgaccaacaaaacttgggatcacttttggttaaatgagggacatactgtgtacttggaacgccacatttgcggacgattgtttggtgaaaagttcagacattttaatgctctgggaggatggggagaactacagaattcggtaaagacatttggggagacacatcctttcaccaaacttgtggttgatctgacagatatagaccctgatgtagcttattcttcagttccctatgagaagggctttgctttacttttttaccttgaacaactgcttggaggaccagagattttcctaggattcttaaaagcttatgttgagaagttttcctataagagcataactactgatgactggaaggatttcctgtattcctattttaaagataaggttgatgttctcaatcaagttgattggaatgcctggctctactctcctggactgcctcccataaagcccaattatgatatgactctgacaaatgcttgtattgccttaagtcaaagatggattactgccaaagaagatgatttaaattcattcaatgccacagacctgaaggatctctcttctcatcaattgaatgagtttttagcacagacgctccagagggcacctcttccattggggcacataaagcgaatgcaagaggtgtacaacttcaatgccattaacaattctgaaatacgattcagatggctgcggctctgcattcaatccaagtgggaggacgcaattcctttggcgctaaagatggcaactgaacaaggaagaatgaagtttacccggcccttattcaaggatcttgctgcctttgacaaatcccatgatcaagctgtccgaacctaccaagagcacaaagcaagcatgcatcccgtgactgcaatgctggtggggaaagacttaaaagtggattaaagacctgcgtattgatgattttagagatttctcttttttaaatggaattcgtaaagaaatataaaacttcagctcacaattaaaactgtctttttagttttggctttttattgttttgttggtgattttactgaaataaagatgagctacttcttc

SEQ ID NO: 43 - DNA, 13432 bp, Homo sapiens
ggaaggggcccggatgtactgaggatgcgttacagtttcactcgaggaggcagtagtggaaaggagcagtttttggggtttgatgccataatgggaatcaggtaatcgtcggcggggaagaagaaacgctgcagaccacggcttcctcgaatcttgcgcgaaagccgccggcctcggaggagggattaatccagacccgccggggggtgttttcacatttcttcctcttcgtggctgctcctcctattaaaaccatttttggtccatggtcaatgagaatacgaggatgtacattccagaggaaaaccaccaaggttccaactatgggagcccacgccccgcccatgccaacatgaatgccaatgcggcagcggggctggcccctgagcacatccccaccccgggggctgccctgtcgtggcaggcggccatcgacgcagcccggcaggctaagctgatgggcagcgctggcaatgcgaccatctccacagtcagctccacgcagcggaagcggcagcaatatgggaaacccaagaagcagggcagcaccacggccacacgcccgccccgagccctgctctgcctgaccctgaagaaccccatccggagggcctgcatcagcattgtcgaatggaaaccatttgaaataattattttactgactatttttgccaattgtgtggccttagcgatctatattccctttccagaagatgattccaacgccaccaattccaacctggaacgagtggaatatctctttctcataatttttacggtggaagcgtttttaaaagtaatcgcctatggactcctctttcaccccaatgcctacctccgcaacggctggaacctactagattttataattgtggttgtggggctttttagtgcaattttagaacaagcaaccaaagcagatggggcaaacgctctcggagggaaaggggccggatttgatgtgaaggcgctgagggccttccgcgtgctgcgccccctgcggctggtgtccggagtcccaagtctccaggtggtcctgaattccatcatcaaggccatggtccccctgctgcacatcgccctgcttgtgctgtttgtcatcatcatctacgccatcatcggcttggagctcttcatggggaagatgcacaagacctgctacaaccaggagggcatagcagatgttccagcagaagatgacccttccccttgtgcgctggaaacgggccacgggcggcagtgccagaacggcacggtgtgcaagcccggctgggatggtcccaagcacggcatcaccaactttgacaactttgccttcgccatgctcacggtgttccagtgcatcaccatggagggctggacggacgtgctgtactgggtcaatgatgccgtaggaagggactggccctggatctattttgttacactaatcatcatagggtcattttttgtacttaacttggttctcggtgtgcttagcggagagttttccaaagagagggagaaggccaaggcccggggagatttccagaagctgcgggagaagcagcagctagaagaggatctcaaaggctacctggattggatcactcaggccgaagacatcgatcctgagaatgaggacgaaggcatggatgaggagaagccccgaaacatgagcatgcccaccagtgagaccgagtccgtcaacaccgaaaacgtggctggaggtgacatcgagggagaaaactgcggggccaggctggcccaccggatctccaagtcaaagttcagccgctactggcgccggtggaatcggttctgcagaaggaagtgccgcgccgcagtcaagtctaatgtcttctactggctggtgattttcctggtgttcctcaacacgctcaccattgcctctgagcactacaaccagcccaactggctcacagaagtccaagacacggcaaacaaggccctgctggccctgttcacggcagagatgctcctgaagatgtacagcctgggcctgcaggcctacttcgtgtccctcttcaaccgctttgactgcttcgtcgtgtgtggcggcatcctggagaccatcctggtggagaccaagatcatgtccccactgggcatctccgtgctcagatgcgtccggctgctgaggattttcaagatcacgaggtactggaactccttgagcaacctggtggcatccttgctgaactctgtgcgctccatcgcctccctgctccttctcctcttcctcttcatcatcatcttctccctcctggggatgcagctctttggaggaaagttcaactttgatgagatgcagacccggaggagcacattcgataacttcccccagtccctcctcactgtgtttcagatcctgaccggggaggactggaattcggtgatgtatgatgggatcatggcttatggcggcccctcttttccagggatgttagtctgtatttacttcatcatcctcttcatctgtggaaactatatcctactgaatgtgttcttggccattgctgtggacaacctggctgatgctgagagcctcacatctgcccaaaaggaggaggaagaggagaaggagagaaagaagctggccaggactgccagcccagagaagaaacaagagttggtggagaagccggcagtgggggaatccaaggaggagaagattgagctgaaatccatcacggctgacggagagtctccacccgccaccaagatcaacatggatgacctccagcccaatgaaaatgaggataagagcccctaccccaacccagaaactacaggagaagaggatgaggaggagccagagatgcctgtcggccctcgcccacgaccactctctgagcttcaccttaaggaaaaggcagtgcccatgccagaagccagcgcgtttttcatcttcagctctaacaacaggtttcgcctccagtgccaccgcattgtcaatgacacgatcttcaccaacctgatcctcttcttcattctgctcagcagcatttccctggctgctgaggacccggtccagcacacctccttcaggaaccatattctgttttattttgatattgtttttaccaccattttcaccattgaaattgctctgaagatgactgcttatggggctttcttgcacaagggttctttctgccggaactacttcaacatcctggacctgctggtggtcagcgtgtccctcatctcctttggcatccagtccagtgcaatcaatgtcgtgaagatcttgcgagtcctgcgagtactcaggcccctgagggccatcaacagggccaaggggctaaagcatgtggttcagtgtgtgtttgtcgccatccggaccatcgggaacatcgtgattgtcaccaccctgctgcagttcatgtttgcctgcatcggggtccagctcttcaagggaaagctgtacacctgttcagacagttccaagcagacagaggcggaatgcaagggcaactacatcacgtacaaagacggggaggttgaccaccccatcatccaaccccgcagctgggagaacagcaagtttgactttgacaatgttctggcagccatgatggccctcttcaccgtctccaccttcgaagggtggccagagctgctgtaccgctccatcgactcccacacggaagacaagggccccatctacaactaccgtgtggagatctccatcttcttcatcatctacatcatcatcatcgccttcttcatgatgaacatcttcgtgggcttcgtcatcgtcacctttcaggagcagggggagcaggagtacaagaactgtgagctggacaagaaccagcgacagtgcgtggaatacgccctcaaggcccggcccctgcggaggtacatccccaagaaccagcaccagtacaaagtgtggtacgtggtcaactccacctacttcgagtacctgatgttcgtcctcatcctgctcaacaccatctgcctggccatgcagcactacggccagagctgcctgttcaaaatcgccatgaacatcctcaacatgctcttcactggcctcttcaccgtggagatgatcctgaagctcattgccttcaaacccaagcactatttctgtgatgcatggaatacatttgacgccttgattgttgtgggtagcattgttgatatagcaatcaccgaggtaaacccagctgaacatacccaatgctctccctctatgaacgcagaggaaaactcccgcatctccatcaccttcttccgcctgttccgggtcatgcgtctggtgaagctgctgagccgtggggagggcatccggacgctgctgtggaccttcatcaagtccttccaggccctgccctatgtggccctcctgatcgtgatgctgttcttcatctacgcggtgatcgggatgcaggtgtttgggaaaattgccctgaatgataccacagagatcaaccggaacaacaactttcagaccttcccccaggccgtgctgctcctcttcaggtgtgccaccggggaggcctggcaggacatcatgctggcctgcatgccaggcaagaagtgtgccccagagtccgagcccagcaacagcacggagggtgaaacaccctgtggtagcagctttgctgtcttctacttcatcagcttctacatgctctgtgccttcctgatcatcaacctctttgtagctgtcatcatggacaactttgactacctgacaagggactggtccatccttggtccccaccacctggatgagtttaaaagaatctgggcagagtatgaccctgaagccaagggtcgtatcaaacacctggatgtggtgaccctcctccggcggattcagccgccactaggttttgggaagctgtgccctcaccgcgtggcttgcaaacgcctggtctccatgaacatgcctctgaacagcgacgggacagtcatgttcaatgccaccctgtttgccctggtcaggacggccctgaggatcaaaacagaagggaacctagaacaagccaatgaggagctgcgggcgatcatcaagaagatctggaagcggaccagcatgaagctgctggaccaggtggtgccccctgcaggtgatgatgaggtcaccgttggcaagttctacgccacgttcctgatccaggagtacttccggaagttcaagaagcgcaaagagcagggccttgtgggcaagccctcccagaggaacgcgctgtctctgcaggctggcttgcgcacactgcatgacatcgggcctgagatccgacgggccatctctggagatctcaccgctgaggaggagctggacaaggccatgaaggaggctgtgtccgctgcttctgaagatgacatcttcaggagggccggtggcctgttcggcaaccacgtcagctactaccaaagcgacggccggagcgccttcccccagaccttcaccactcagcgcccgctgcacatcaacaaggcgggcagcagccagggcgacactgagtcgccatcccacgagaagctggtggactccaccttcaccccgagcagctactcgtccaccggctccaacgccaacatcaacaacgccaacaacaccgccctgggtcgcctccctcgccccgccggctaccccagcacggtcagcactgtggagggccacgggccccccttgtcccctgccatccgggtgcaggaggtggcgtggaagctcagctccaacaggtgccactcccgggagagccaggcagccatggcgggtcaggaggagacgtctcaggatgagacctatgaagtgaagatgaaccatgacacggaggcctgcagtgagcccagcctgctctccacagagatgctctcctaccaggatgacgaaaatcggcaactgacgctcccagaggaggacaagagggacatccggcaatctccgaagaggggtttcctccgctctgcctcactaggtcgaagggcctccttccacctggaatgtctgaagcgacagaaggaccgagggggagacatctctcagaagacagtcctgcccttgcatctggttcatcatcaggcattggcagtggcaggcctgagccccctcctccagagaagccattcccctgcctcattccctaggccttttgccaccccaccagccacacctggcagccgaggctggcccccacagcccgtccccaccctgcggcttgagggggtcgagtccagtgagaaactcaacagcagcttcccatccatccactgcggctcctgggctgagaccacccccggtggcgggggcagcagcgccgcccggagagtccggcccgtctccctcatggtgcccagccaggctggggccccagggaggcagttccacggcagtgccagcagcctggtggaagcggtcttgatttcagaaggactggggcagtttgctcaagatcccaagttcatcgaggtcaccacccaggagctggccgacgcctgcgacatgaccatagaggagatggagagcgcggccgacaacatcctcagcgggggcgccccacagagccccaatggcgccctcttaccctttgtgaactgcagggacgcggggcaggaccgagccgggggcgaagaggacgcgggctgtgtgcgcgcgcggggtcgaccgagtgaggaggagctccaggacagcagggtctacgtcagcagcctgtagtgggcgctgccagatgcgggcttttttttatttgtttcaatgttcctaatgggttcgtttcagaagtgcctcactgttctcgtgacctggagttaaccggaacagcgtcttcattcatttctgttgggaccagacgcggagcctgggtgcgcgagccgccctccgggaggaaggcgcccggctgcgtctgcagaggcggggagaggaggcggcgagggtcccggggcgcgaggaaggcgcctgccctctcccagctcgcaggccccgggcccggccgcgcctccgcggggagagcaccccggcttcccgcgcgccctcaccaaaaggaccctacagcaaacgggtgtctttcgactctgcttgtagaaaccatttgcacatattctgtacgagcctcgctgtctccctagagccagggccctgcggatttggagaagggagcggggcaggacttccaggaggaccccaacccggcccggagagggaggaggaggcctccaggggcgcggagctctggggatgggcgtcgggccggcagtggtgcggctcactccgtccctgcccacctgcgacgggatcccccgaccggcacgggccacgccgagctcccggccagccgccggcccgcaggcagcgcgagggaggagctgcgccgccggctccgcccaaccaggtggtgctgagcttccgctgagcgctcttttgttttgtggtttgacacttttcttgacagcatgttgcagtttcttttcggttttggttttttttaaatgttttattttgctttcccagcgggaggggaggaagaagagtgtttacaaagtcctgtagccccctcacctttctgttttcacttttgccaatgtacatcgggtttggttttcttgtattatttaaacggttgtggtttcctttttccacggaggttcaatagaagccgctgcaggagagttttaccaaccattgtgtatgcccaataatttgttatcatttccttaggtagtaacctatttttgttctggtttggttcggttatctaatggaaaggtaactggcaatgcacttgatgtggtcttgcacatgtgggtgatagagttgggttcctttttatgctgggtgtacaggtgggtttgggagagaggagcatgcgcgagagagtctccgagtgtgtgcgacgcgtgtgtgtgtggtgggttgtctgtgtgcatatgtcctgcccgtgtatatgcacccacaccatgtgcccgtgcacaccagtgactacgcagtcccccctttctggtttagctgtgggaagatctgaatctggggccgtttgaaagcaaaaacaaaccactgtctctgcttctgaaacgggaatcagtaactctttgcattttctgtcccacaagatatgcaaaaacaatgcaataatattcatttaaaaatacaattgtgagttgtgttggcattaaaactgtattttaaaaaaagacagaaatttaagggaaaaacacaagaaggcattttgcttcaatatattccgtgtaatgttttattgcattgataatgtttctgttgaagaaaccgttatacttgaattcaggtcagtttcagtatttttcaaatatttttttaaaactgaattgcaattgtgccaagcgaatataatgaattgaattaagttggttttcggattcacttcttgtatattttgctgcatgtaaagtaaatcattttgtatttggagtgtgacaagctttacctttgaactcaagtgcttttctatatgtggttgggggaaagggaacaagttttctttagtttgcacaatgagcaaaggtatcaccagtgtagtcattattctgctctccacaaacaggtttggacattactgttttgcatatcttgtgtttgcttacatttccctcaatttttccaaaatcgtttgctgggtatgtttgtaccgcctcttgctgtgagagaccaggacctattttattccagtcttcactctgtccactctgctctggtcatctgatttggtacttctccaagaacagcccttcactgtgaggtgcagggaggcgttctgatgagccctcagtcactgggccgtcatccgcatcccccatggaagaggtagctggctttcccttcccttccaccacacggaattttctctttggcttccttaggaaagtgtacactaaccgggaggataaaattaaagtcaggctgcttggagggaggggcatcctcacttccggattcttgttgctctacccaacaaggacagcaggggctcgagaaaggaactggtgaaaccctgatccatctgaaagtcaactctgcgtgctcctttctccatcccttcctcactctggagcagcctttccttcaggcttgccctaatgtttgggctgccggggagggggccaggacaagggaagaggcatccggagctcacagtgggggtgggaacagatttttgtgggggcatctctaatgctcacttatatctccctagaacatcactcttttggtgctgtgtccttcaaatgtatgtcaacagtggtggctgaaaagggactgctttggggaaaacaggacccaaccattcacccagaattgacccattaaatctcttccagtcctagtgttccctgagcccctcttggcacatatataagtaagctagaaattacaataagggacagtccattcctctatgacagcttgctggactgattcatgacaaagtggagaaatgtactcaatactccccggttaacacagtctagaaacagagtttctttatggatatccacacccaagtcatccaaactttcttgattccttttcactgccatcaaggtcctctagaaattgagtttaggtatcatcctttgaaaagttcccaagatttctaccaggaggtacacacaggcgttccctgtctagggcaggaggactatcctagcttgaccttctgatccactagaataagactggcgtatgatgcctgtcatcagaacagactggcacaagtagtgacatcaatgaaccacagcacaatcttccaagtgatgtctactctccacctaaaatggaattttccccatgaccttgtaaaacataattgtcacatcttccatacccctcctgacagcccccaagtgtcaggagaaaacagtcaggggctaagggcccaagggacttgaagaaacaacagtttaaggtctgcagtttggtcaacttaattcttgtcctccgaccagccctgcctctttcatttccagaccttggagaatttttcccagctttgattcagaaggtactagttataacccctttccttcttcttaatccaataggcctcactctcactgggaaatccactcaaaggaacaaggcaatgtctctcattctatttcccagttccaaattccaggtgcttgtctggagtgaagctacccgttactttctcccagcttttctccacccagcatgtctcctgcccatgcagctgaagacagtggggcaacctcaggagaagcagacctttccatgcccaagttcatctcctgagcaacagtgacacctagaaaatgaggactttggaagtcacccaaaagatggtggctactttatggagtcctgaagatacacagccaccactcctaaaggcaaagaaagaaaacacgaatgtaggtcagggatagagtggaaccctggtcatcggggtttttagcctcatcgtgggaaaggtggtaaaggaggatgatggcatctccatccctagaggccaagaattgaaatatcattgtcaaggattagaaacaattcagcaaagaggccacaaaaagggcctgctgactcccagaagacctctttaaaccccaggggaggcaaatacttgctgatggagtctgggccgtttccatattttaaagaagacctgcctctggggcaaatgtcagcacagagaggactgggaggagaatggaggcaagaaaagggcatattttgactccctctgtgcctcttcccagttcatggaaggatgtgttcagcttacccacccacagtgaccagtgtggtggagccgctgacatctcaaggatctatttgggaaggtgagaagagtactcattccatctgggggtgttgttccagccacatcagcctacctggtgggatgtgggggtgtctgccaccctgtcccccctctgctgatgtccctcccctcaggctgtccaggtgccacctgacacaggctgctgtgcaaagacaggcggggaagcccaaacctcactcccagggaggccctcagccgccagagtccaggttctccagaggctacgatttgaggaggttgagggggaagacaggagggaaagaaaagtcctacaactgtcaggaatggggcacctttccctgtccctaagcaaagctccctcttcccactgccctccccagccccagctccctgtcctccccaacacctagtgagaaagacggtgcgtggaagggagtcccatgggcagatgcttacacgacctctttgtgaagcctcttctgggtttaacttcattcatcaatttattcttatgtcaaagcaatgaaacttttctttctggagccagataccaatacaacaggtgaacgggtttctgccacatctctacattgacgggggatgcttgaacaacccccctcactacacagacacacaccgttaaggcacaagggctggggttgagctctagatgagggactttcctgctcctgcaagggtgagcactgtatacacagacaaggagggtgcagtagagtgactcccttggatggaagtagtaccatcagaacctactattattatgacataaattctatttacatacattgagagaatactacaatcaacactttttcctgggatgactttaagaggtttgagccacagcacctgaagtggcaaagatccatggtctttgtagggtattagagaactcttccagtcacctctgaaagcactctagatcttgcagctgagtggatgaagtgtaacaaatctgttgcacgctgagaggagtcagaattagcatttttcatgaaagttccccacgtctctactaagaatgaggaagaaaagactaagactaggtaattacacagaggcttgaaatgttacatcaccagagccaagtcctctcccttcagatcagttactggctgctacacagggacacccccaccttttcagggcatcccatgcactccacttctcaggatctaaggaatttgactttgtagggatcccagaaagggcactgtgccacttcccctggtgtgaatcagacatacattgtacattcatttctaaaattcactcatgcacctcaaaccaaggtcattatccaaaaaaaaaaaaaaaaagctctgggtggaagagtttgtaagttttaagagagggtcatttctatgtgaggaaatgcagaaatggacagaatgattcttattcactgtttgggtctggagaattcccattgtggaaatcttagagatctcaagtttattaccaagggaataaggaaaaaaaggtgagcaggcaccaggccaagcagtggctcccttgccaaggaacctgaggctgcaggtttcagggacccccttgaagaaacctctcctggccattggccaggagaaaagagaagtctctcctgtagagtcacaagagaagcaaaagagggtgggtcactgggtcctggacatagcccccaaccccaagacttcccaatatggagaaactacaccaatgtttaaaaggggaaaaggaaagaacttgtacatcaagggaagatgatttgtaaacacacagtcctgtgcagaaagatccccttcaggaggtgtctccagcatcccaaagctgtgcgcaccttctcttttcctgcctcaggccacctatgcatccagctgcagcccatacccacacctgaaatccatctcttgaatcccagccaggttatataccaccccattgccatgtcctgtcctggccagaatgcatgctgttcccccaagcctcgtgggagtgaggccatgggaaacagagatgagcatgtctggacaagtctgtgatggtagtggatgagaataacccatggcaaaacacgcacattcattaagaaatagggcgacagattccccgttggtgaagcactgaaaggtctattactctcataatattgctgttttattttaatccaccagagctaccatgcaaaactttcctcctgtgaaacgctccagataaagctcctctaatctccccttccctcatgtcctccagctcaaacccaccttcatcccccaaaccaatctgtatcatgcctgttatcagagaggcacagaaagatgggcagtgcctccgttgtcaccattccccacacccctacacacccccacaccctcccctccaggctccacgacttcacagtcttactgttgtaaatatcattgtacagtttgtaatcctcaaataatcccattgtcagaggcctcgcctgatgggccttctcaccctcgagaaaggccagggaatctagaaggggcaacccttcaaggagagcttcagggtcatctctgtgtgagacactattgtatattcctgtaagattgcatttttatctaaggaatgatgttatttaaaaaacaaacaaaaaacacaaaaaataagaattgcaaataaatttcttaacaatgtct

SEQ ID NO: 44 - DNA, 1839 bp, Homo sapiens
atgaaggccacctggatcaggcttctgaaaagagccaagggaggaaggctgaagaattctgatatctgtggttcggcagactcctacactagccgtccatccgattccgatgtatctctggaggaggaccgggaggcagtgcgcagagaagcggagcggcaggcccaggcacagttggaaaaagcaaagacaaagcccgttgcatttgcggttcggacaaatgtcagctacagtgcggcccatgaagatgatgttccagtgcctggcatggccatctcattcgaagcaaaagattttctgcatgttaaggaaaaatttaacaatgactggtggatagggcgattggtaaaagaaggctgtgaaatcggattcattccaagcccagtcaaactagaaaacatgaggctgcagcatgaacagagagccaagcaagggaaattctactccagtaaatcaggaggaaattcatcatccagtttgggtgacatagtacctagttccagaaaatcaacacctccatcatctgctatagacatagatgctactggcttagatgcagaagaaaatgatattccagcaaaccaccgctcccctaaacccagtgcaaacagtgtaacgtcaccccactccaaagagaaaagaatgcccttctttaagaagacagagcacactcctccgtatgatgtggtaccttccatgcgaccagtggtcctagtgggcccttctctgaagggctacgaggtcacagatatgatgcaaaaagcgctgtttgattttttaaaacacagatttgaagggcggatatccatcacaagggtcaccgctgacatctcgcttgccaaacgctcggtattaaacaatcccagtaagcacgcaataatagaaagatccaacacaaggtcaagcttagcggaagttcagagtgaaatcgaaaggatttttgaacttgcaagaacattgcagttggtggtccttgacgcggatacaattaatcatccagctcaactcagtaaaacctccttggcccctattatagtatatgtaaagatttcttctcctaaggttttacaaaggttaataaaatctcgagggaaatctcaagctaaacacctcaacgtccagatggtagcagctgataaactggctcagtgtcctccagagctgttcgatgtgatcttggatgagaaccagcttgaggatgcctgtgagcaccttgccgactatctggaggcctactggaaggccacccatcctcccagcagtagcctccccaaccctctccttagccgtacattagccacttcaagtctgcctcttagccccaccctagcctctaattcacagggttctcaaggtgatcagaggactgatcgctccgctcctatccgttctgcttcccaagctgaagaagaacctagtgtggaaccagtcaagaaatcccagcaccgctcttcctcctcagccccacaccacaaccatcgcagtgggacaagtcgcggcctctccaggcaagagacatttgactcggaaacccaggagagtcgagactctgcctacgtagagccaaaggaagattattcccatgaccacgtggaccactatgcctcacaccgtgaccacaaccacagagacgagacccacgggagcagtgaccacagacacagggagtcccggcaccgttcccgggacgtggatcgagagcaggaccacaacgagtgcaacaagcagcgcagccgtcataaatccaaggatcgctactgtgaaaaggatggagaagtgatatcaaaaaaacggaatgaggctggggagtggaacagggatgtttacatcccccaatga

SEQ ID NO: 45 - DNA, 873 bp, Homo sapiens
acttccccttcctgtacagggcaggttgtgcagctggaggcagagcagtcctctctggggagcctgaagcaaacatggatcaagaaactgtaggcaatgttgtcctgttggccatcgtcaccctcatcagcgtggtccagaatggattctttgcccataaagtggagcacgaaagcaggacccagaatgggaggagcttccagaggaccggaacacttgcctttgagcgggtctacactgccaaccagaactgtgtagatgcgtaccccactttcctcgctgtgctctggtctgcggggctactttgcagccaagttcctgctgcgtttgctggactgatgtacttgtttgtgaggcaaaagtactttgtcggttacctaggagagagaacgcagagcacccctggctacatatttgggaaacgcatcatactcttcctgttcctcatgtccgttgctggcatattcaactattacctcatcttctttttcggaagtgactttgaaaactacataaagacgatctccaccaccatctcccctctacttctcattccctaactctctgctgaatatggggttggtgttctcatctaatcaatacctacaagtcatcataattcagctcttgagagcattctgctcttctttagatggctgtaaatctattggccatctgggcttcacagcttgagttaaccttgcttttccgggaacaaaatgatgtcatgtcagctccgccccttgaacatgaccgtggccccaaatttgctattcccatgcattttgtttgtttcttcacttatcctgttctctgaagatgttttgtgaccaggtttgtgttttcttaaaataaaatgcagagacatgtttt

(19) United States
(12) Patent Application Publication
(10) Pub. No.: US 2010/0021903 A1
(43) Pub. Date: Jan. 28, 2010

(54) Use of Genetic Determinants in Cardiovascular Risk Assessment

(75) Inventors: Julie Johnson, Melrose, FL (US); Amber L. Beitelshees, Gainesville, FL (US); Michael Pacanowski, Gainesville, FL (US); Harm Knot, Gainesville, FL (US)

Correspondence Address: EDWARDS ANGELL PALMER & DODGE LLP, P.O. BOX 55874, BOSTON, MA, 02205, US

(73) Assignee: University of Florida Research Foundation, Inc., Gainesville, FL (US)

(21) Appl. No.: 12/466,895

(22) Filed: May 15, 2009

Related U.S. Application Data
(63) Continuation of application No. PCT/US2007/024068, filed on Nov. 15, 2007.
(60) Provisional application No. 60/859,370, filed on Nov. 15, 2006.

(51) Int. Cl. C12Q 1/68; C40B 40/08; C07H 21/04

(52) U.S. Cl. 435/6; 506/17; 536/24.33

(57) ABSTRACT

The invention generally provides compositions and methods of using a subject's genetic information for the selection of prophylactic or therapeutic agents and treatment regimens, and related methods for assaying the risk of an adverse cardiovascular event in the patient.

### CROSS-REFERENCE TO RELATED APPLICATION

This application claims the benefit of U.S. Provisional Application No. 60/859,370, filed Nov. 15, 2006, the entire contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: R01 HL74730. The government may have certain rights in the invention.

### BACKGROUND OF THE INVENTION

Human genetic variation underlies disparities in how individual patients respond to drug therapies. Progress in pharmacogenetics suggests that a variety of genetic and environmental factors produce variability in drug responsiveness. Analyzing a patient's genotype at one or more genetic loci that are correlated with drug responsiveness can provide for the individualization of treatment regimens and streamline the identification of effective therapies. In hypertension, for example, data from the National Health and Nutrition Examination Survey show that only 56% of treated hypertensive patients have their blood pressure under control despite the availability of many drug classes from which to choose therapy. This suggests that the current empirical approach to identifying appropriate drug therapy for individual patients is not working. If those with difficult-to-control hypertension or those susceptible to adverse cardiovascular outcomes could be identified earlier, more aggressive therapies could be pursued at the time that treatment is initiated. In addition, those with hypertension amenable to hypertensive monotherapy could also be identified, lessening the likelihood of polypharmacy. Improved methods for assessing adverse cardiovascular risk and selecting hypertensive therapy are required.

### SUMMARY OF THE INVENTION

As described below, the present invention generally provides compositions and methods of using a patient's genetic information for the selection of prophylactic or therapeutic agents and treatment regimens, and related methods for assaying the risk of an adverse cardiovascular event in the patient.

In a first aspect, the invention generally provides a method of identifying a subject (e.g., human or veterinary patient) as having a propensity to have an adverse cardiovascular event comprising analysing a subject sample for an alteration in the nucleic acid sequence of any one or more of CACNA1C, CACNB2, ALOX5, α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), or arachidonate 5-lipoxygenase-activating protein (ALOX5AP) nucleic acid molecule gene relative to a wild-type reference sequence, the presence or absence of the alteration indicating propensity to have an adverse cardiovascular event. In one embodiment, the alteration is in one, two, three, four, or five of these genes. In one embodiment, the alteration identifies the subject as having an increased propensity to have an adverse cardiovascular event (e.g., a fatal or nonfatal myocardial infarction or a stroke). In one embodiment, the alteration in the ADD1 gene is ADD1 460Trp polymorphism. In another embodiment, the alteration in the β1-adrenergic receptor gene is the 46G-79C-523C haplotype. In yet another embodiment, the alteration in the β1-adrenergic receptor gene is the 49S-389R haplotype. In still another embodiment, the alteration in the β2-adrenergic receptor gene variants is the 46G-79G-523C haplotype. In still another embodiment, the alteration in the LTA4H gene variant is the rs2247570 TT and rs1978331 AA. In still another embodiment, the alteration identifies the subject as having a reduced propensity to have an adverse cardiovascular event. In still another embodiment, the alteration in the KCNMB1 gene is the Lys65 variant or the Leu110 variant. In yet another embodiment, the alteration in the ALOX5AP gene is the GTC haplotype.

In another aspect, the invention provides a method for identifying a subject as having an allele variant correlated with increased or decreased efficacy of a treatment. The method involves analyzing a nucleic acid sample obtained from said subject to determine whether said sample comprises at least one allele variant in any one or more of the following genes: CACNA1C, CACNB2, ALOX5, α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), and arachidonate 5-lipoxygenase-activating protein (ALOX5AP) gene relative to a wild-type reference sequence, the variant being correlated to increased or decreased responsiveness to a treatment for a cardiovascular disorder. In one embodiment, the treatment is β-blocker therapy. In another embodiment, the treatment is verapamil SR therapy. In yet another embodiment, the allele variant Lys65 in the calcium activated potassium channel (KCNMB1) gene identifies the subject as responsive to verapamil therapy. In yet another embodiment, the allele variant Leu110 in the calcium activated potassium channel (KCNMB1) gene identifies the subject as responsive to a calcium channel blocker. In still another embodiment, the allele variants 46G-79C-523C in the β2-adrenergic receptor identifies responsiveness to verapamil therapy. In another embodiment, the absence of the allele variants 46G-79C-523C in the β2-adrenergic receptor gene identifies responsiveness to atenolol therapy. In yet another embodiment, the allele variants 49S-389R in the β1-adrenergic receptor gene identifies responsiveness to β-blocker therapy. In still another embodiment, the homozygous allele variants AA in the LTA4H gene identifies responsiveness to atenolol therapy. In still another embodiment, the G-allele variant in the LTA4H gene identifies equal responsiveness to verapamil SR or atenolol therapy.

In a related aspect, the invention provides a method for identifying a cardiovascular therapy for a subject. The method involves analyzing a nucleic acid sample obtained from said subject to determine whether the sample comprises at least one allele variant correlated to increased or decreased responsiveness to a treatment for a cardiovascular condition; and selecting a treatment as a treatment regimen for the subject having said allele. In one embodiment, the allele variant Lys65 in the calcium activated potassium channel (KCNMB1) gene identifies verapamil therapy as a treatment regimen for the subject. In another embodiment, the allele variant Leu110 in the calcium activated potassium channel (KCNMB1) gene identifies a calcium channel blocker therapy as a treatment regimen for the subject. In still another embodiment, the allele variants 46G-79C-523C in the β2-adrenergic receptor identifies verapamil therapy as a treatment regimen for the subject. In still another embodiment, the absence of the allele variants 46G-79C-523C in the β2-adrenergic receptor gene identifies atenolol therapy as a treatment regimen for the subject. In still another embodiment, the allele variants 49S-389R in the β1-adrenergic receptor gene identifies β-blocker therapy as a treatment regimen for the subject. In still another embodiment, the homozygous allele variants AA in the LTA4H gene identifies atenolol therapy as a treatment regimen for the subject. In still another embodiment, the G-allele variant in the LTA4H gene identifies verapamil SR or atenolol therapy as a treatment regimen for the subject.

In another related aspect, the invention provides a method for identifying a subject in need of early and aggressive cardiovascular therapy. The method involves analyzing a nucleic acid sample obtained from the subject to determine whether said sample comprises at least one allele variant correlated to a propensity to have an adverse cardiovascular event, thereby identifying the subject as in need of early and aggressive cardiovascular therapy. In one embodiment, the method further involves identifying a cardiovascular disease risk factor selected from the group consisting of age, race, sex, body mass index, blood pressure, smoking status, low density lipid (LDL) or high density lipid (HDL) cholesterol level, systolic blood pressure, dyastolic blood pressure, history of heart failure, diabetes, renal insufficiency, or left ventricular hypertrophy, alcohol consumption history, smoking history, exercise history, diet, and family history of cardiovascular disease.

In another related aspect, the invention provides a method for determining a subject's propensity for an adverse cardiovascular event. The method involves determining, in a sample from said subject, the presence or absence of an allelic variant in a gene that is any one or more of CACNA1C, CACNB2, ALOX5, α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), and arachidonate 5-lipoxygenase-activating protein (ALOX5AP) gene; assessing age, race, sex, body mass index, blood pressure, smoking status, low density lipid (LDL) or high density lipid (HDL) cholesterol level, systolic blood pressure, dyastolic blood pressure, history of heart failure, diabetes, renal insufficiency, or left ventricular hypertrophy, alcohol consumption history, smoking history, exercise history, diet, and family history of cardiovascular disease; and correlating the presence of the allele variant and one or more cardiovascular disease risk factors with the need for early and aggressive cardiovascular therapy, where a positive correlation identifies the subject as having a propensity for an adverse cardiovascular event. In one embodiment, the method further involves selecting an appropriate treatment regimen. In another embodiment, the method further involves administering the treatment regimen to the subject.

In yet another aspect, the invention provides a method for identifying a subject as in need of prophylactic cardiovascular therapy. The method involves determining, in a sample from said subject, the presence or absence of an allelic variant in a gene that is any one or more of CACNA1C, CACNB2, ALOX5, α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), and arachidonate 5-lipoxygenase-activating protein (ALOX5AP) gene; assessing age, race, sex, body mass index, blood pressure, smoking status, low density lipid (LDL) or high density lipid (HDL) cholesterol level, systolic blood pressure, dyastolic blood pressure, history of heart failure, diabetes, renal insufficiency, or left ventricular hypertrophy, alcohol consumption history, smoking history, exercise history, diet, and family history of cardiovascular disease; and correlating the presence of the allele variant and one or more cardiovascular disease risk factors with the need for prophylactic cardiovascular therapy.

In yet another aspect, the invention provides a nucleic acid microarray contains a nucleic acid molecule containing at least a fragment of two, three, four, or more of the following genes CACNA1C, CACNB2, ALOX5, α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), and arachidonate 5-lipoxygenase-activating protein (ALOX5AP) gene. In one embodiment, the nucleic acid sequences comprise sequences for at least two different alleles for each of said genes.

In yet another aspect, the invention provides a primer set containing primers that bind to two or more genes that is any one or more of CACNA1C, CACNB2, ALOX5, α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), and arachidonate 5-lipoxygenase-activating protein (ALOX5AP) gene. In one embodiment, at least one primer binds differentially to two different alleles of one of said genes.

In yet another aspect, the invention provides a kit for determining the presence of absence of an allelic variant in a biological sample from a subject, the kit containing a nucleic acid sequence that hybridizes to a gene that is any one or more of α-adducin (ADD1) gene, calcium activated potassium channel (KCNMB1) gene, 1-adrenergic receptor (ADRB1) gene, β2-adrenergic receptor (ADRB2) gene, leukotriene A4 hydrolase (LTA4H), and arachidonate 5-lipoxygenase-activating protein (ALOX5AP) gene. In one embodiment, the method further comprises determining one or more factors selected from the group consisting of the subject's age, race, sex, body mass index, blood pressure, smoking status, low density lipid (LDL) or high density lipid (HDL) cholesterol level, systolic blood pressure, dyastolic blood pressure, history of heart failure, diabetes, renal insufficiency, or left ventricular hypertrophy.

In another aspect, the invention provides a method for selecting a cardiovascular therapy for a subject, the method comprising identifying a subject as having or lacking an allelic variant in CACNA1C (e.g., a SNP identified by NCBI Ref Seq rs10848683 or rs1051375).

In another aspect, the invention provides a method for selecting a cardiovascular therapy for a subject, the method comprising identifying a subject as having or lacking an allelic variant in CACNB2. In one embodiment, the method identifies the presence or absence of a single nucleotide polymorphism identified by NCBI Ref Seq: rs120036.

In another aspect, the invention provides a method for selecting a cardiovascular therapy for a subject, the method comprising identifying a subject as having or lacking an allelic variant in ALOX5 (e.g., a tandem repeat promoter polymorphism).

In various embodiments, the therapy selected is beta blocker therapy (e.g., atenolol).

In various embodiments of any previous aspect, the method further involves reporting test results to a subject or to a health provider. In various embodiments of any previous aspect, the test results are used to select a method of treatment.

In another aspect, the invention provides a representation (e.g., represented on a computer screen, a database, or a paper or other print out) of the test results of any previous aspect. In one embodiment, the representation further contains information relating to selection of a treatment or prophylactic method.

In another aspect, the invention provides a pharmaceutical composition comprising an effective amount of a therapeutic agent (e.g., a beta blocker, such as atenolol) labeled for use in a patient selected by the method of any previous aspect.

In various embodiments of any of the above aspects, the method further involves determining one or more factors selected from the group consisting of the subject's age, race, sex, body mass index, blood pressure, smoking status, low density lipid (LDL) or high density lipid (HDL) cholesterol level, systolic blood pressure, dyastolic blood pressure, history of heart failure, diabetes, renal insufficiency, or left ventricular hypertrophy. In other embodiments of any of the above aspects, the sample is an oral tissue sample (e.g., a buccal swab, scraping, or wash) or biological fluid sample (e.g., saliva, urine, or blood). In other embodiments of any of the above aspects, the presence or absence of the allele variant is identified by amplifying or failing to amplify an amplification product from the sample. In still other embodiments of any of the above aspects, the amplification product is digested with a restriction enzyme before analysis. In still other embodiments of any of the above aspects, the presence or absence of the allele variant is identified by hybridizing the nucleic acid sample with a primer labeled with a detectable moiety. In other embodiments of any of the above aspects, the detectable moiety is detected in an enzymatic assay, radioassay, immunoassay, or by detecting fluorescence. In other embodiments of any of the above aspects, the primer is labeled with a detectable dye (e.g., SYBR Green I, YO-PRO-1, thiazole orange, Hex, pico green, edans, fluorescein, FAM, or TET). In other embodiments of any of the above aspects, the primers are located on a chip. In other embodiments of any of the above aspects, the primers for amplification are specific for alleles of the genes.

The invention provides a pharmacogenetics-based approach that uses genetic information to assess a patient's risk of an adverse cardiovascular outcome and to select antihypertensive agents and treatment regimens for the patient. Other features and advantages of the invention will be apparent from the detailed description, and from the claims.

### DEFINITIONS

By “agent” is meant any small molecule chemical compound, antibody, nucleic acid molecule, or polypeptide, or fragments thereof.

By “adverse cardiovascular event” is meant a primary clinical outcome. Exemplary adverse cardiovascular events include stroke, fatal or non-fatal myocardial infarction, or cardiovascular-related death.

By “allele variant” is meant any alteration in a nucleic acid sequence relative to a wild-type reference sequence. Allele variants include polymorphisms, such as single nucleotide polymorphisms and restriction fragment length polymorphisms. Alterations in nucleic acid sequence may be in a coding or non-coding region of a gene. Such alterations in nucleotide sequence need not result in a change in the amino acid sequence of an encoded protein.

By “analysing” or “testing” is meant any method for detecting a genetic mutation in a polynucleotide or polypeptide. Such methods include, but are not limited to, direct sequencing, hybridization, amplification, or any other method of detecting an alteration in an amino acid or nucleic acid sequence.

By “α-adducin polypeptide” is meant a protein having at least 85% identity to the amino acid sequence provided at NCBI Reference Sequence No. NP_{—}001110 and having blood pressure modulating function. An exemplary α-adducin amino acid sequence is provided below.

α-adducin NP_{—}001110.

| | |
|---|---|
| 1 | mngdsraavv tspppttaph keryfdrvde nnpeylrern mapdlrqdfn mmeqkkrvsm |
| 61 | ilqspafcee lesmiqeqfk kgknptglla lqqiadfmtt nvpnvypaap qggmaalnms |
| 121 | lgmvtpvndl rgsdsiaydk gekllrckla afyrladlfg wsqliynhit trvnseqehf |
| 181 | livpfgllys evtasslvki nlqgdivdrg stnlgvnqag ftlhsaiyaa rpdvkcvvhi |
| 241 | htpagaavsa mkcgllpisp ealslgevay hdyhgilvde eekvliqknl gpkskvlilr |
| 301 | nhglvsvges veeafyyihn lvvaceiqvr tlasaggpdn lvllnpekyk aksrspgspv |
| 361 | gegtgsppkw qigeqefeal mrmldnlgyr tgypyrypal rekskkysdv evpasvtgys |
| 421 | fasdgdsgtc splrhsfqkq qrektrwlns grgdeaseeg qngsspkskt kwtkedghrt |
| 481 | stsavpnlfv plntnpkevq emrnkireqn lqdiktagpq sqvlcgvvmd rslvqgelvt |
| 541 | askaiiekey qphvivsttg pnpfttltdr eleeyrreve rkqkgseenl deareqkeks |
| 601 | ppdqpavphp ppstpiklee dlvpepttgd dsdaatfkpt lpdlspdeps ealgfpmlek |
| 661 | eeeahrppsp teapteaspe papdpapvae eaapsaveeg aaadpgsdgs pgkspskkkk |
| 721 | kfrtpsflkk skkksds |

By “α-adducin (ADD1) gene” is meant a nucleic acid molecule encoding an α adducin polypeptide. An exemplary α-adducin sequence is provided at NCBI Reference Sequence No. NM_{—}001119, which is hereby incorporated by reference in its entirety.

By “calcium activated potassium channel polypeptide” is meant a protein having at least 85% identity to the amino acid sequence provided at NCBI Reference Sequence No. NP_{—}004128 and having potassium conducting activity. An exemplary calcium activated potassium channel amino acid sequence is provided below.

Calcium activated potassium channel polypeptide; NCBI Reference Sequence No. NP_{—}004128

| | |
|---|---|
| 1 | mvkklvmaqk rgetralclg vtmvvcavit yyilvttvlp lyqksvwtqe skchlietni |
| 61 | rdqeelkgkk vpqypclwvn vsaagrwavl yhtedtrdqn qqcsyipgsv dnyqtaradv |
| 121 | ekvrakfqeq qvfycfsapr gnetsvlfqr lygpqallfs lfwptflltg glliiamvks |
| 181 | nqylsilaaq k. |

By “calcium activated potassium channel (KCNMB1) nucleic acid molecule” is meant a polynucleotide encoding a calcium activated potassium channel. An exemplary calcium activated potassium channel (KCNMB1) nucleic acid molecule is provided at NM_{—}004137, which is hereby incorporated by reference in its entirety.

By “CACNA1C” is meant a protein having at least 85% identity to the amino acid sequence provided at NCBI Reference Sequence No. Q13936 and having calcium conducting activity. An exemplary Voltage-dependent L-type calcium channel subunit alpha-1C (CACNA1C) amino acid sequence is provided below.

CACNA1C polypeptide; NCBI Reference No. 013936:

| | |
|---|---|
| 1 | mvnentrmyi peenhqgsny gsprpahanm nanaaaglap ehiptpgaal swqaaidaar |
| 61 | qaklmgsagn atistvsstq rkrqqygkpk kqgsttatrp prallcltlk npirracisi |
| 121 | vewkpfeiii lltifancva laiyipfped dsnatnsnle rveylfliif tveaflkvia |
| 181 | ygllfhpnay lrngwnlldf iivvvglfsa ileqatkadg analggkgag fdvkalrafr |
| 241 | vlrplrlvsg vpslqvvlns iikamvpllh iallvlfvii iyaiiglelf mgkmhktcyn |
| 301 | qegiadvpae ddpspcalet ghgrqcqngt vckpgwdgpk hgitnfdnfa famltvfqci |
| 361 | tmegwtdvly wvndavgrdw pwiyfvtlii igsffvlnlv lgvlsgefsk erekakargd |
| 421 | fqklrekqql eedlkgyldw itqaedidpe nedegmdeek prnmsmptse tesvntenva |
| 481 | ggdiegencg arlahrisks kfsrywrrwn rfcrrkcraa vksnvfywlv iflvflntlt |
| 541 | iasehynqpn wltevqdtan kallalftae mllkmyslgl qayfvslfnr fdcfvvcggi |
| 601 | letilvetki msplgisvlr cvrllrifki trywnslsnl vasllnsvrs iaslllllfl |
| 661 | fiiifsllgm qlfggkfnfd emqtrrstfd nfpqslltvf qiltgedwns vmydgimayg |
| 721 | gpsfpgmlvc iyfiilficg nyillnvfla iavdnladae sltsaqkeee eekerkklar |
| 781 | taspekkqel vekpavgesk eekielksit adgesppatk inmddlqpne nedkspypnp |
| 841 | ettgeedeee pempvgprpr plselhlkek avpmpeasaf fifssnnrfr lqchrivndt |
| 901 | iftnlilffi llssislaae dpvqhtsfrn hilfyfdivf ttiftieial kilgnadyvf |
| 961 | tsiftleiil kmtaygaflh kgsfcrnyfn ildllvvsvs lisfgiqssa invvkilrvl |
| 1021 | rvlrplrain rakglkhvvq cvfvairtig nivivttllq fmfacigvql fkgklytcsd |
| 1081 | sskqteaeck gnyitykdge vdhpiiqprs wenskfdfdn vlaammalft vstfegwpel |
| 1141 | lyrsidshte dkgpiynyrv eisiffiiyi iiiaffmmni fvgfvivtfq eqgeqeyknc |
| 1201 | eldknqrqcv eyalkarplr ryipknqhqy kvwyvvnsty feylmfvlil lnticlamqh |
| 1261 | ygqsclfkia mnilnmlftg lftvemilkl iafkpkgyfs dpwnvfdfli vigsiidvil |
| 1321 | setnhyfcda wntfdalivv gsivdiaite vnpaehtqcs psmnaeensr isitffrlfr |
| 1381 | vmrlvkllsr gegirtllwt fiksfqalpy vallivmlff iyavigmqvf gkialndtte |
| 1441 | inrnnnfqtf pqavlllfrc atgeawqdim lacmpgkkca pesepsnste getpcgssfa |
| 1501 | vfyfisfyml cafliinlfv avimdnfdyl trdwsilgph hldefkriwa eydpeakgri |
| 1561 | khldvvtllr riqpplgfgk lcphrvackr lvsmnmplns dgtvmfnatl falvrtalri |
| 1621 | ktegnleqan eelraiikki wkrtsmklld qvvppagdde vtvgkfyatf liqeyfrkfk |
| 1681 | krkeqglvgk psqrnalslq aglrtlhdig peirraisgd ltaeeeldka mkeavsaase |
| 1741 | ddifrraggl fgnhvsyyqs dgrsafpqtf ttqrplhink agssqgdtes psheklvdst |
| 1801 | ftpssysstg snaninnann talgrlprpa gypstvstve ghgpplspai rvqevawkls |
| 1861 | snrerhvpvc edlelrrdsg sagtqahcll lrranpsrch sresqaamag qeetsqdety |
| 1921 | evkmnhdtea csepsllste mlsyqddenr qltlpeedkr dirqspkrgf lrsaslgrra |
| 1981 | sfhleclkrq kdrggdisqk tvlplhlvhh qalavaglsp llqrshspas fprpfatppa |
| 2041 | tpgsrgwppq pvptlrlegv esseklnssf psihcgswae ttpggggssa arrvrpvslm |
| 2101 | vpsqagapgr qfhgsasslv eavliseglg qfaqdpkfie vttqeladac dmtieemesa |
| 2161 | adnilsggap qspngallpf vncrdaggdr aggeedagcv rargapseee lqdsrvyvss |
| 2221 | l |

By “CACNA1C nucleic acid molecule” is meant a polynucleotide encoding a CACNA1C polypeptide or a variant of the polynucleotide. Exemplary CACNA1C nucleic acid sequences useful as references include the following: NM_{—}000719, BC093695, which are hereby incorporated by reference in its entirety.

By “CACNB2 polypeptide” is meant a protein having at least 85% amino acid sequence identity to NCBI Reference No. NP_{—}000715 and having calcium conducting activity. The amino acid sequence of NP_{—}000715 calcium channel, voltage-dependent, beta-2 subunit is provided below:

| | |
|---|---|
| 1 | mqccglvhrr rvrvsygsad sytsrpsdsd vsleedreav rreaerqaqa qlekaktkpv |
| 61 | afavrtnvsy saaheddvpv pgmaisfeak dflhvkekfn ndwwigrlvk egceigfips |
| 121 | pvklenmrlq heqrakqgkf yssksggnss sslgdivpss rkstppssai didatgldae |
| 181 | endipanhrs pkpsansvts phskekrmpf fkktehtppy dvvpsmrpvv lvgpslkgye |
| 241 | vtdmmqkalf dflkhrfegr isitrvtadi slakrsvlnn pskhaiiers ntrsslaevq |
| 301 | seierifela rtlqlvvlda dtinhpaqls ktslapiivy vkisspkvlq rliksrgksq |
| 361 | akhlnvqmva adklaqcppe lfdvildenq ledacehlad yleaywkath ppssslpnpl |
| 421 | lsrtlatssl plsptlasns qgsqgdqrtd rsapirsasq aeeepsvepv kksqhrssss |
| 481 | aphhnhrsgt srglsrqetf dsetqesrds ayvepkedys hdhvdhyash rdhnhrdeth |
| 541 | gssdhrhres rhrsrdvdre qdhnecnkqr srhkskdryc ekdgeviskk rneagewnrd |
| 601 | vyirq |

By “CACNB2 nucleic acid molecule” is meant a polynucleotide encoding a CACNB2 polypeptide or variant thereof. Exemplary CACNB2 nucleic acid sequences useful as references include the following: NCBI Accession No. AF423192, AH010523, each of which are hereby incorporated by reference in their entirety.

By “ALOX5 polypeptide” is meant a polypeptide having at least 85% amino acid sequence identity to the amino acid sequence provided at NCBI Reference No. NP_{—}000689.

The amino acid sequence of NP_{—}000689 is provided below:

| | |
|---|---|
| 1 | mpsytvtvat gsqwfagtdd yiylslvgsa gcsekhlldk pfyndferga vdsydvtvde |
| 61 | elgeiqlvri ekrkywlndd wylkyitlkt phgdyiefpc yrwitgdvev vlrdgrakla |
| 121 | rddqihilkq hrrkeletrq kqyrwmewnp gfplsidakc hkdlprdiqf dsekgvdfvl |
| 181 | nyskamenlf inrfmhmfqs swndfadfek ifvkisntis ervmnhwqed lmfgyqflng |
| 241 | cnpvlirrct elpeklpvtt emvecslerq lsleqevqqg nifivdfell dgidanktdp |
| 301 | ctlqflaapi cllyknlank ivpiaiqlnq ipgdenpifl psdakydwll akiwvrssdf |
| 361 | hvhqtithll rthlvsevfg iamyrqlpav hpifkllvah vrftiaintk areqlicecg |
| 421 | lfdkanatgg gghvqmvqra mkdltyaslc fpeaikargm eskedipyyf yrddgllvwe |
| 481 | airtftaevv diyyegdqvv eedpelqdfv ndvyvygmrg rkssgfpksv ksreqlseyl |
| 541 | tvviftasaq haavnfgqyd wcswipnapp tmrappptak gvvtieqivd tlpdrgrscw |
| 601 | hlgavwalsq fqenelflgm ypeehfiekp vkeamarfrk nleaivsvia ernkkkqlpy |
| 661 | yylspdripn svai |

By ALOX5P nucleic acid molecule” is meant a polynucleotide encoding a ALOX5P polypeptide or variant thereof. An exemplary ALOX5P nucleic acid sequences useful as a reference includes NM_{—}000698, which is hereby incorporated by reference in its entirety.

By “cardiovascular condition” is meant a disorder of the cardiovascular system. Non-limiting examples of cardiovascular conditions include atherosclerosis, primary myocardial infarction, secondary myocardial infarction, angina pectoris (including both stable and unstable angina), congestive heart failure, sudden cardiac death, cerebral infarction, restenosis, syncope, ischemia, reperfusion injury, vascular occlusion, carotid obstructive disease, transient ischemic attack, and the like.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “Beta1-adrenergic receptor (ADRB1) polypeptide” is meant a protein having at least 85% amino acid sequence identity to NCBI Reference Sequence No. NP_{—}000675 that binds epinephrine or norepinephrine. An exemplary Beta1-adrenergic receptor amino acid sequence is provided below.

Beta-1-adrenergic receptor [Homo sapiens]. NP_{—}000675

| | |
|---|---|
| 1 | mgagvlvlga sepgnlssaa plpdgaataa rllvpasppa sllppasesp eplsqqwtag |
| 61 | mgllmalivl livagnvlvi vaiaktprlq tltnlfimsl asadlvmgll vvpfgativv |
| 121 | wgrweygsff celwtsvdvl cvtasietlc vialdrylai tspfryqsll trararglvc |
| 181 | tvwaisalvs flpilmhwwr aesdearrcy ndpkccdfvt nrayaiassv vsfyvplcim |
| 241 | afvylrvfre aqkqvkkids cerrflggpa rppspspspv papapppgpp rpaaaaatap |
| 301 | langragkrr psrlvalreq kalktlgiim gvftlcwlpf flanvvkafh relvpdrlfv |
| 361 | ffnwlgyans afnpiiycrs pdfrkafqgl lccarraarr rhathgdrpr asgclarpgp |
| 421 | ppspgaasdd ddddvvgatp parllepwag cnggaaadsd ssldepcrpg faseskv |

By “Beta1-adrenergic receptor (ADRB1) nucleic acid molecule” is meant a polynucleotide encoding a beta1-adrenergic receptor. An exemplary β1-adrenergic nucleic acid sequence is provided at NCBI Reference Sequence No. NM_{—}000684.

By “Beta2-adrenergic receptor (ADRB2) polypeptide” is meant a protein having at least 85% identity to the amino acid sequence provided at NCBI Reference Sequence No. AAB82151 and having norepinephrine or epinephrine binding activity. An exemplary beta2-adrenergic receptor amino acid sequence is provided below.

Beta2-adrenergic receptor [Homo sapiens]. AAB82151

| | |
|---|---|
| 1 | mgqpgngsaf llapnrshap dhdvtqqrde vwvvgmgivm slivlaivfg nvlvitaiak |
| 61 | ferlqtvtny fitslacadl vmglavvpfg aahilmkmwt fgnfwcefwt sidvlcvtas |
| 121 | ietlcviavd ryfaitspfk yqslltknka rviilmvwiv sglisflpiq mhwyrathqe |
| 181 | aincyanetc cdfftnqaya iassivsfyv plvimvfvys rvfqeakrql qkidksegrf |
| 241 | hvqnlsqveq dgrtghglrr sskfclkehk alktlgiimg tftlcwlpff ivnivhviqd |
| 301 | nlirkevyil lnwigyvnsg fnpliycrsp dfriafqell clrrsslkay gngyssngnt |
| 361 | geqsgyhveq ekenkllced lpgtedfvgh qgtvpsdnid sqgrncstnd sll |

By “Beta2-adrenergic receptor (ADRB2) nucleic acid molecule” is meant a polynucleotide encoding a beta2-adrenergic receptor polypeptide. An exemplary beta2-adrenergic receptor nucleic acid molecule is provided at NCBI Reference Sequence No AF022956, which is hereby incorporated by reference in its entirety.

By “arachidonate 5-lipoxygenase-activating protein” is meant a protein having at least 85% identity to the amino acid sequence provided at NCBI Reference Sequence No. NP_{—}001620 and having 5-lipoxygenase enzyme activating activity. An exemplary arachidonate 5-lipoxygenase-activating protein amino acid sequence is provided below.

| | |
|---|---|
| 1 | mdqetvgnvv llaivtlisv vqngffahkv ehesrtqngr sfqrtgtlaf ervytanqnc |
| 61 | vdayptflav lwsagllcsq vpaafaglmy lfvrqkyfvg ylgertqstp gyifgkriil |
| 121 | flflmsvagi fnyylifffg sdfenyikti sttispllli p |

By “arachidonate 5-lipoxygenase-activating nucleic acid molecule” is meant a polynucleotide encoding a arachidonate 5-lipoxygenase-activating protein. An exemplary arachidonate 5-lipoxygenase-activating nucleic acid molecule is provided at NCBI Reference Sequence No. NM_{—}001629, which is hereby incorporated by reference in its entirety.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “early and aggressive cardiovascular therapy” is meant a treatment approach that aims to control blood pressure rapidly, which for some individuals will require the use of multiple antihypertensive drugs. If desirable, such therapy may also include glucose control, weight loss control, and smoking cessation.

By “leukotriene A4 hydrolase polypeptide” is meant a protein having at least 85% identity to the amino acid sequence provided at NCBI Reference Sequence No. NP_{—}000886 and having hydrolase enzymatic activity. An exemplary leukotriene A4 hydrolase sequence is provided below.

Leukotriene A4 hydrolase [Homo sapiens]. NP_{—}000886

| | |
|---|---|
| 1 | mpeivdtcsl aspasvcrtk hlhlrcsvdf trrtltgtaa ltvqsqednl rslvldtkdl |
| 61 | tiekvvingq evkyalgerq sykgspmeis lpialsknqe ivieisfets pkssalqwlt |
| 121 | peqtsgkehp ylfsqcqaih crailpcqdt psvkltytae vsvpkelval msairdgetp |
| 181 | dpedpsrkiy kfiqkvpipc ylialvvgal esrqigprtl vwsekeqvek sayefsetes |
| 241 | mlkiaedlgg pyvwgqydll vlppsfpygg menpcltfvt ptllagdksl snviaheish |
| 301 | swtgnlvtnk twdhfwlneg htvylerhic grlfgekfrh fnalggwgel qnsvktfget |
| 361 | hpftklvvdl tdidpdvays svpyekgfal lfyleqllgg peiflgflka yvekfsyksi |
| 421 | ttddwkdfly syfkdkvdvl nqvdwnawly spglppikpn ydmtltnaci alsqrwitak |
| 481 | eddlnsfnat dlkdlsshql netlaqtlqr aplplghikr mqevynfnai nnseirfrwl |
| 541 | rlciqskwed aiplalkmat eqgrmkftrp lfkdlaafdk shdqavrtyq ehkasmhpvt |
| 601 | amlvgkdlkv d |

By “leukotriene A4 hydrolase gene” is meant a gene encoding an leukotriene A4 hydrolase polypeptide. The sequence of an exemplary LTA4H gene is provided at NCBI Reference Sequence No. NM_{—}000895.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. And S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “increased or decreased responsiveness to a treatment” is meant having an enhanced or reduced reaction to therapy. Where a subject has increased responsiveness to therapy, a reduced level of a therapeutic agent may be required, or a given amount produces an enhanced therapeutic outcome relative to a control condition. Where a subject has a reduced responsiveness to therapy, an increased amount or number of therapeutic agents is required to achieve a given therapeutic effect.

By “responsive” when used in the context of a patient administered a therapeutic agent is meant having greater than 50% probability that the agent will produce a beneficially therapeutic effect in the patient. Preferably, the probability that the agent will produce a beneficial therapeutic effect is at least about 75%, 85%, or 100%.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “genetic information” is meant knowledge related to the genotype of a subject. In particular, information related to polymorphisms, including single nucleotide polymorphisms, in the nucleic acid sequence of one or both chromosomes.

By “hybridize” is meant pair to form a double-stranded molecule between complementary polynucleotide sequences, or portions thereof, under various conditions of stringency.

By “marker” is meant any protein or polynucleotide having an alteration in expression level or activity that is associated with a disease or disorder.

By “metric” is meant a measure. A metric may be used, for example, to correlate a cardiovascular disease or the propensity to develop a cardiovascular disease with a variant allele of a nucleic acid molecule of interest. In other embodiments, the metric may be used to correlate the cardiovascular disease or propensity to develop the cardiovascular disease with a variant allele of a nucleic acid molecule of interest together with other risk factors. Exemplary metrics include, but are not limited to, mathematical formulas or algorithms, such as ratios. The metric to be used is that which best discriminates between differing alleles in a subject having a cardiovascular disease and a normal control subject. Depending on the metric that is used, the diagnostic indicator of cardiovascular disease may be significantly above or below a reference value (e.g., from a control subject not having the variant allele).

“Microarray” means a collection of nucleic acid molecules or polypeptides from one or more organisms arranged on a solid support (for example, a chip, plate, or bead).

By “polymorphism” is meant a genetic variation, mutation, deletion or addition in an α-adducin (ADD1), calcium activated potassium channel (KCNMB1), β1-adrenergic receptor (ADRB1), β2-adrenergic receptor (ADRB2), or leukotriene A4 hydrolase (LTA4H) nucleic acid molecule that is indicative of a predisposition to develop a cardiovascular disease. A polymorphism may be present in the promoter sequence, an open reading frame, intronic sequence, or untranslated 3′ region of an gene.

By “propensity” is meant a predisposition to a condition. Subjects having an increased propensity for a disease or clinical outcome are at increased risk of the disease or outcome.

By “reference” is meant a standard or control condition.

By “substantially identical” is meant a exhibiting at least 50% identity to a reference sequence. Preferably, such a sequence is at least 60%, more preferably 80% or 85%, and most preferably 90%, 95% or even 99% identical at the amino acid level or nucleic acid to the sequence used for comparison.

By “treatment regimen” is meant the methods used to achieve a desirable physiological effect in a subject. Such methods include, but are not limited to, the selection and administration of therapeutic or prophylactic agents or combinations thereof, dosages, dose frequency, and modes of administration.

By “variant carrier” is meant a subject having at least one nucleic acid alteration relative to a reference sequence. Exemplary nucleic acid alterations include the presence of Trp460 in at least one copy of an ADD1 gene.

### BRIEF DESCRIPTION OF THE DRAWINGS

FIG. 1 presents a Kaplan Meier Curve for primary outcome event by ADD1 variant carrier status.

FIG. 2 presents a Kaplan Meier Curve for primary outcome event by ADD1 variant carrier status and diuretic use. The term “Trp460” indicates an ADD1 variant carrier; Gly460Gly indicates ADD1 wild type homozygote; Diuretic indicates use of diuretic; No Diuretic indicates that no diuretic was used.

FIG. 3 shows the effect of ADD1 variant carrier status on primary outcome by subgroups of patients at baseline. Legend: P for interaction (Black vs. not Black)=0.09; P>0.30 for all other interaction terms. *Data not shown for 226 patients with ethnicity=‘other’.

FIG. 4 presents a Kaplan-Meier curves of time to sustained blood pressure control by KCNMB1 codon 65 genotype in verapamil SR monotherapy patients. Sustained blood pressure control was defined as the time point at which blood pressure control (<140/90 mmHg) was achieved and maintained for at least 50% of subsequent visits. Log Rank p=0.01.

FIG. 5 shows the odds ratios for a Lys65 variant on probability of requiring multiple drugs to achieve blood pressure control. Model 1 is adjusted for age, race, and sex. Model 2 is adjusted for all model 1 variables plus baseline systolic blood pressure and dyastolic blood pressure, body mass index (BMI), and history of heart failure, diabetes, renal insufficiency, and left ventricular hypertrophy. FIG. 5A presents results with verapamil SR monotherapy patients and FIG. 5B presents stable background therapy patients.

FIG. 6 presents a Kaplan-Meier curve of time to primary outcome event by Val110Leu variant carrier status in all INternational VErapamil SR-trandolapril STudy (INVEST)-GENES cohort. Black line represents Leu110 carriers, gray line represents Val110Val; log rank p=0.047

FIG. 7 presents a Kaplan-Meier curve of time to primary outcome event by Val110Leu and treatment strategy in all INternational VErapamil SR-trandolapril STudy (INVEST)-GENES cohort. CCB indicates verapamil SR-based strategy; BB indicates atenolol-based strategy; Leu car. indicates Leu 110 variant carrier.

FIG. 8 shows an association between Val110Leu variant carrier status and primary outcome in subgroups

FIG. 9 shows allelic RNA ratios for KCNMB1 measured with marker SNP Val110Leu in heterozygous human heart tissues. Allelic expression ratios of DNA and RNA were measured with SNaPshot assay. RNA ratios were normalized by that of DNA, which were set to 1. Allelic DNA ratio is 1±0.13 (SD). A: major allele, B: minor allele.

FIG. 10 is a graph showing the variability of the antihypertensive drug response.

FIG. 11 is a schematic diagram showing the β1-adrenergic receptor in the lipid bilayer and the response to epinephrine (E) or norepinephrine (NE) binding. The meaning of abbreviations used in the figure is as follows: Gsa denotes the alpha-subunit of guanine nucleotide-binding stimulatory protein of adenylyl cyclase; AC denotes adenylate cyclase; cAMP denotes cyclic adenosine monophosphate; PKA denotes protein kinase A; LTCC denotes L type calcium channels; SR denotes sarcoplasmic reticulum; SERCA2A denotes cardiac sarcoplasmic reticulum Ca²⁺-ATPase; PLB denotes phospholamban

FIG. 12 is a schematic diagram that outlines the functional effects of ADRB1 variants in vitro. The Ser49Gly results in reduced N-glycosylation, agonist affinity, and basil activity, resulting in greater agonist-mediated downregulation of the receptor. The Arg389Gly allele results in reduced Gs coupling, lower basal and agonist-stimulated adenylyl cyclase activity.

FIG. 13 is a graph showing the functional effects of ADRB1 variants ex vivo.

FIG. 14 shows the functional effects of ADBR1 haplotypes in vitro.

FIG. 15 shows the blood pressure response to metoprolol by ADRB1 diplotype.

FIG. 16 is a schematic diagram showing treatment strategies for verapamil SR and atenolol.

FIG. 17 shows primary outcome results for atenolol or verapamil SR treatment strategies as a function of cumulative survival. Primary outcomes include all-cause mortality, nonfatal myocardial infarction, and nonfatal strokes.

FIG. 18 lists the baseline characteristics of the INVEST study population.

FIG. 19 shows pie charts that provide the breakdown of allelic variation in Ser49Gly and Arg389Gly in ADRB1 by ethnic/racial group.

FIG. 20 shows pie charts that provide the breakdown of allelic variation in patients carrying Ser49Gly and Arg389Gly in ADRB1 by ethnic/racial group.

FIG. 21 is a cumlative survival chart of primary outcomes by treatment strategy.

FIG. 22 depicts secondary outcomes by treatment strategy. Secondary outcomes include all-cause mortality, cardiovascular mortality, nonfatal stroke, total stroke, nonfatal myocardial infarction.

FIG. 23 shows blood pressure as a function of treatment strategy over the course of 24 months.

FIG. 24 is a table that shows hazard ratios and primary outcome correlated with ethnic group, clinical condition and treatment strategy.

FIG. 25 is a table showing the effect of ADRB1 haplotype on primary outcome as a function of Hazard ration. SR denotes β₁-AR variant 49S-389R haplotype; SG denotes Ser49 389Gly haplotype; GR denotes 49Gly-389Arg haplotype.

FIG. 26 depicts the effect of the ADRB1 variant 49S-389R haplotype on secondary outcomes. In particular, carriers of the ADRB1 variant 49S-389R haplotype are at higher risk for cardiovascular mortality, total myocardial infarction (fatal and nonfatal), and all cause mortality

FIG. 27 is a graph showing the cumulative survival from primary outcome of carriers of the ADRB1 variant 49S-389R haplotype relative to non-carriers.

FIG. 28 is a graph showing the cumulative survival from all-cause mortality of carriers of the ADRB1 variant 49S-389R haplotype relative to non-carriers.

FIG. 29 is a graph showing the cumulative survival from all-cause mortality of carriers of the ADRB1 variant 49S-389R haplotype as a function of treatment with verapamil or atenolol relative to non-carriers treated with verapamil or atenolol.

FIG. 30 depicts the effect of the ADRB1 variant 49S-389R haplotype on secondary outcomes as a function of treatment strategy. In particular, carriers of the ADRB1 variant 49S-389R haplotype are at higher risk for cardiovascular mortality, total myocardial infarction (fatal and nonfatal), and all cause mortality

FIG. 31 shows covariate-adjusted blood pressure response to 6 weeks of atenolol monotherapy by ADRB2 haplotype Gly16-Glu27-523C (G-E-C). (error bar: standard error of the means)

FIG. 32 shows the adjusted odds ratios for ADRB2 haplotype 16G-E27-523C on the probability of needing more antihypertensive drugs to reach blood pressure control.

FIG. 33 provides exemplary nucleotide sequences of CACNA1C, CACNB2, ALOX5, α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), or arachidonate 5-lipoxygenase-activating protein (ALOX5AP). Such sequences can serve as wild-type reference sequences.

FIG. 34 depicts ADRB1 145A-1 165C haplotype associations with secondary outcomes. Abbreviations: HR, hazard ratio; 95% CI, 95% confidence interval.

FIG. 35 provides a graph and a table showing all-cause mortality and mean blood pressures at end of follow-up by ADRB1 haplotype and atenolol/verapamil SR therapy. Abbreviations: AC, 145A-1 165C haplotype; HR, hazard ratio; 95% CI, 95% confidence interval; AT, atenolol; VE, verapamil SR; SBP, systolic blood pressure; DBP, diastolic blood pressure Hazard ratios based on reduced model adjusted for age, sex, race/ethnicity.

FIG. 36 is a graph that provides a pharmacogenetic analysis of common ADRB2 haplotypes.

FIG. 37 shows that two SNPs (rs1051375 and rs10848683) were associated with significantly better outcomes with β-blocker therapy. For rs1051375, there was a highly significant gene*treatment interaction (p<0.0007).

FIG. 38 shows that ADRB1 codon 389 genotype influences the risk for development of diabetes during antihypertensive therapy.

### DETAILED DESCRIPTION OF THE INVENTION

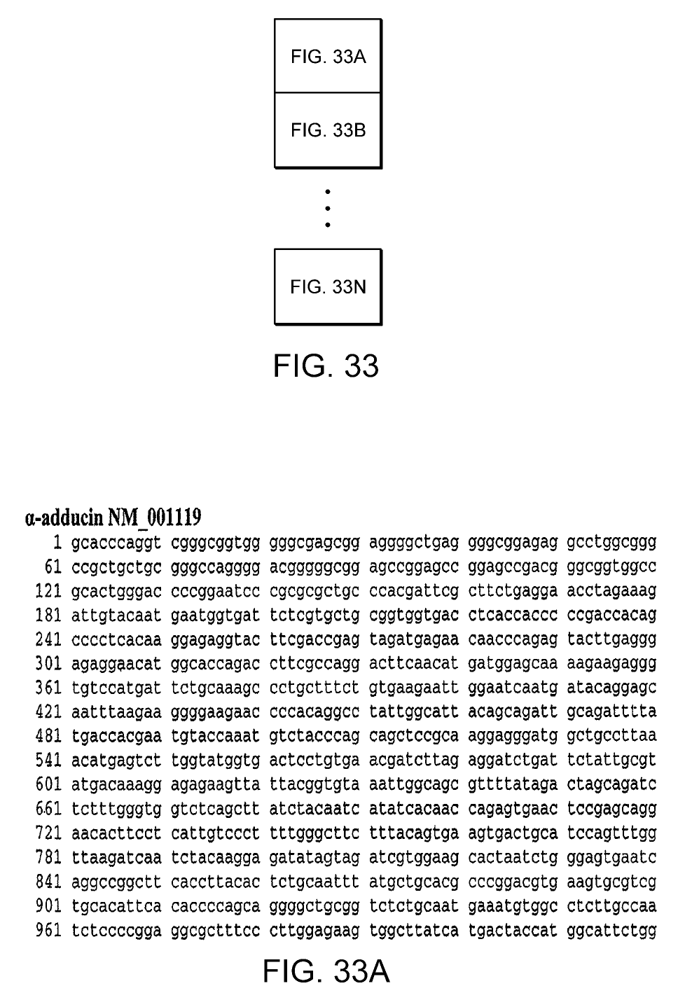

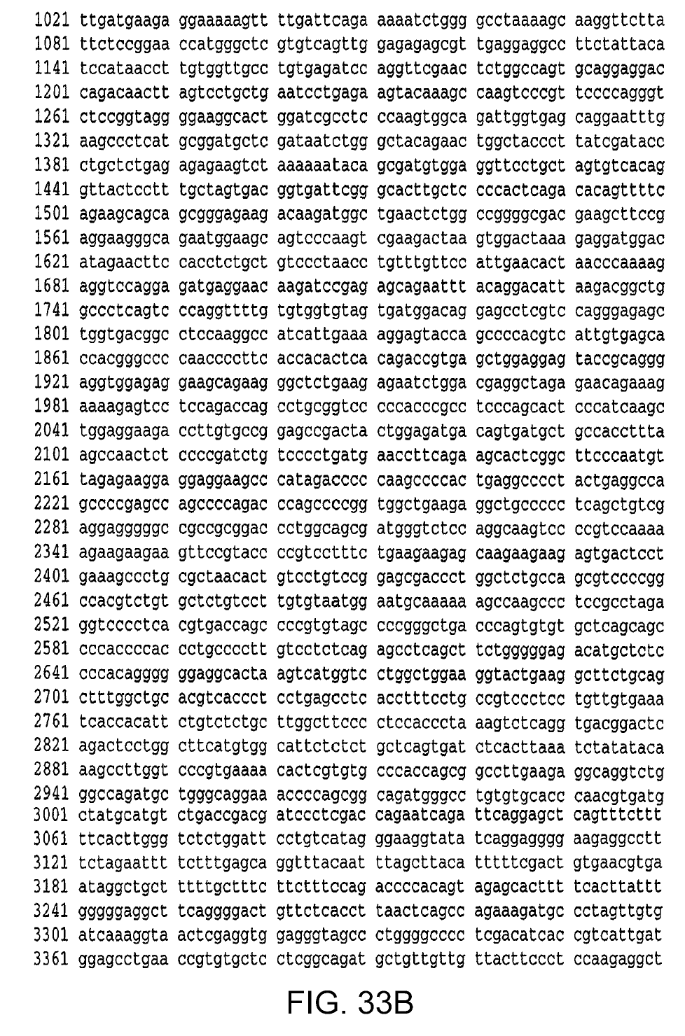

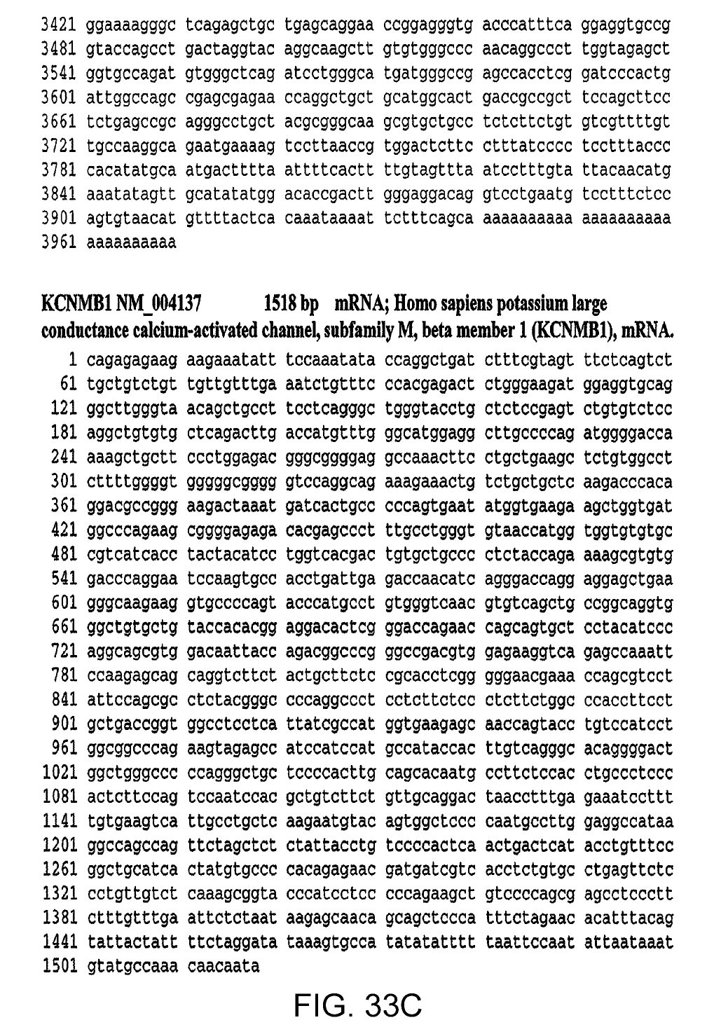

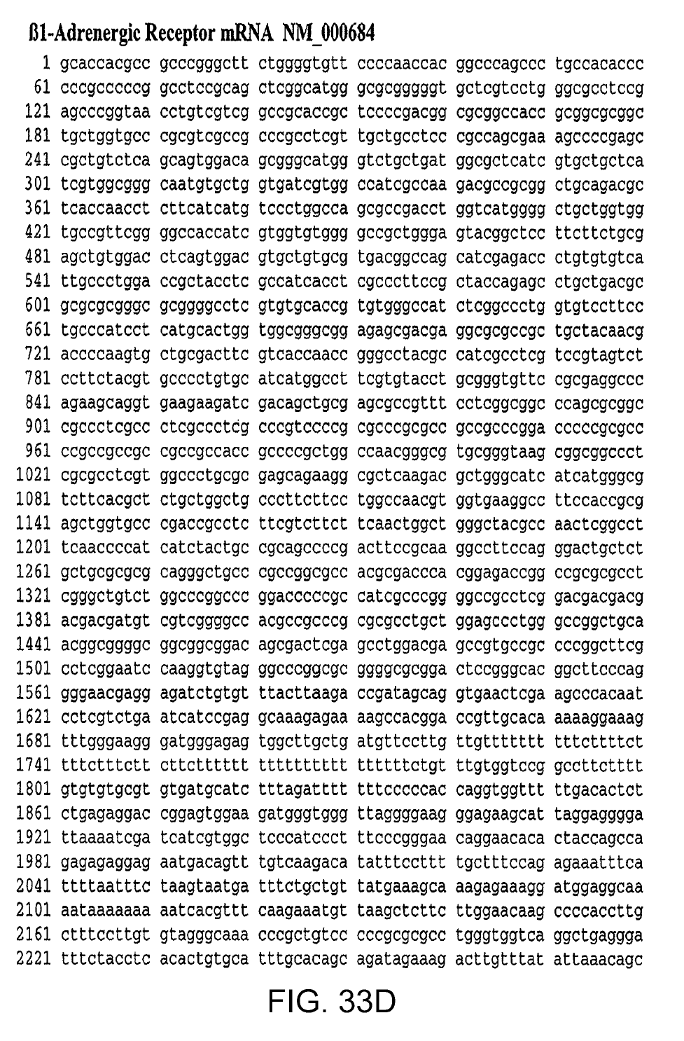

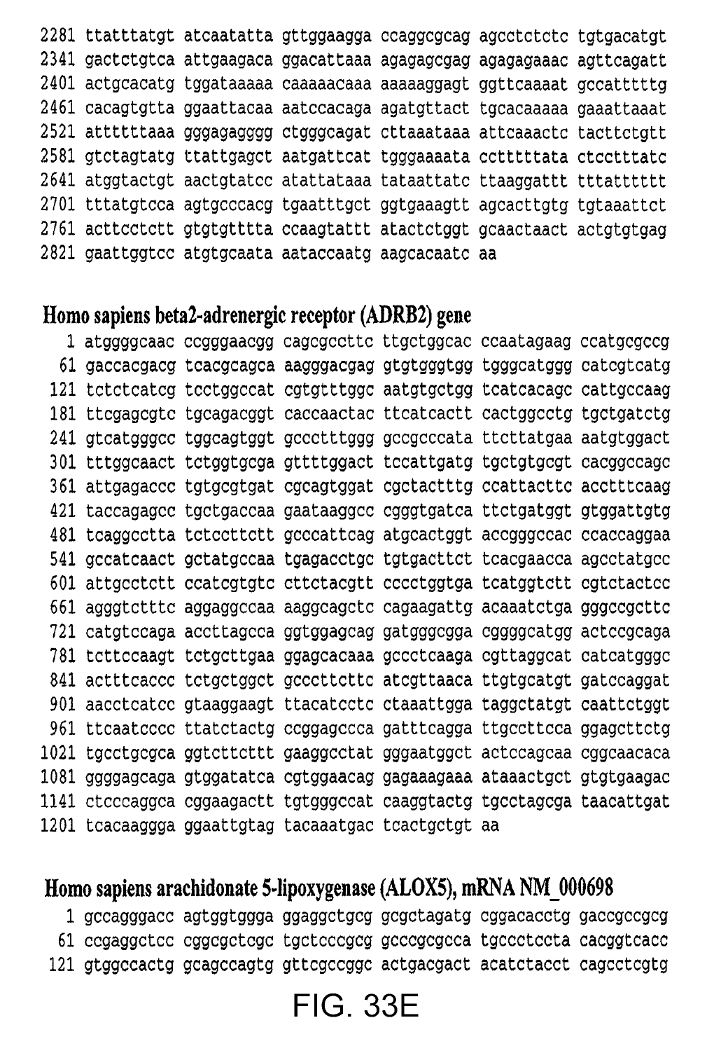

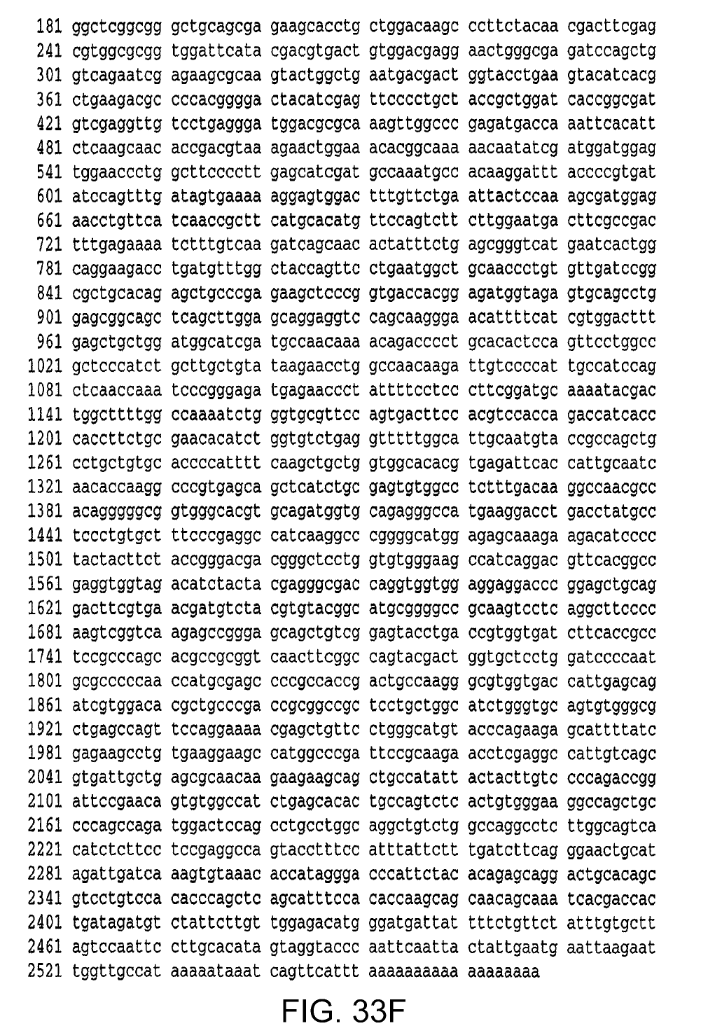

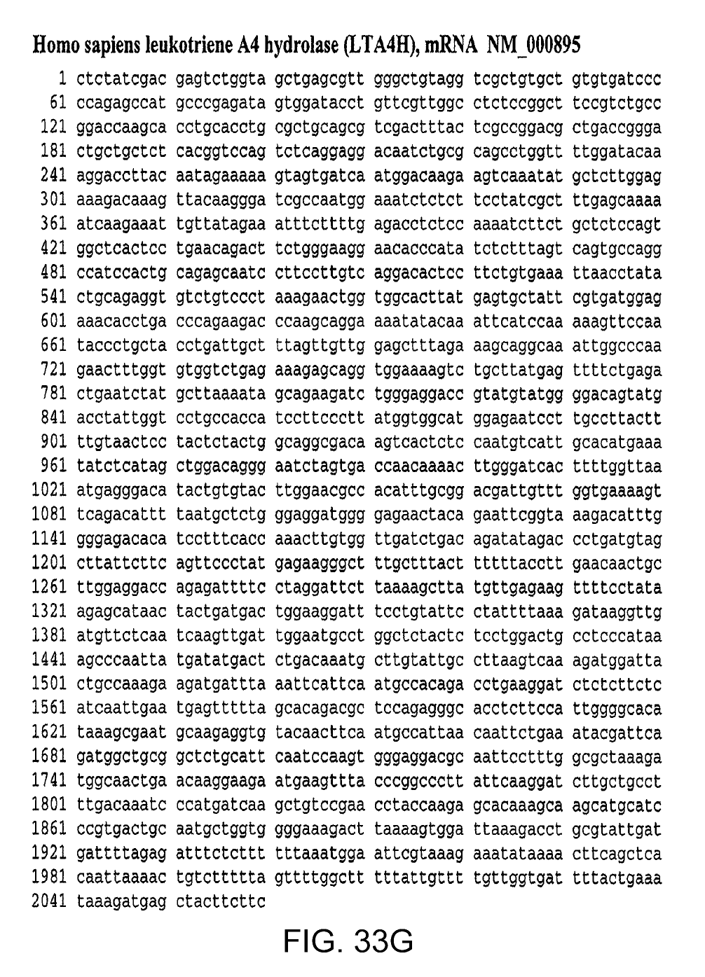

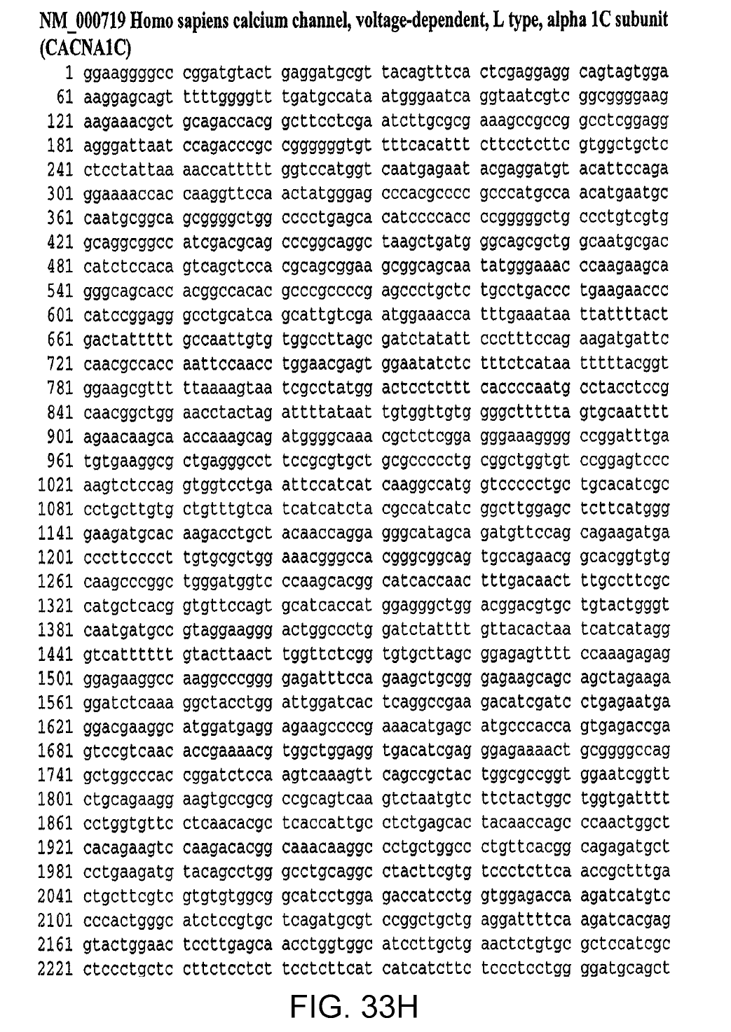

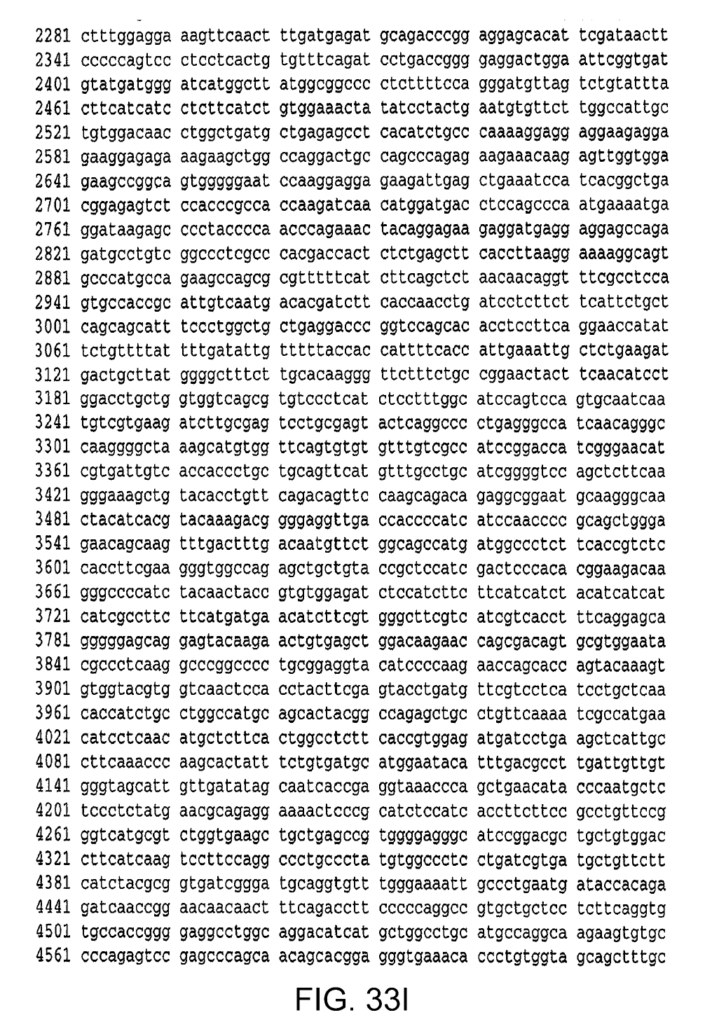

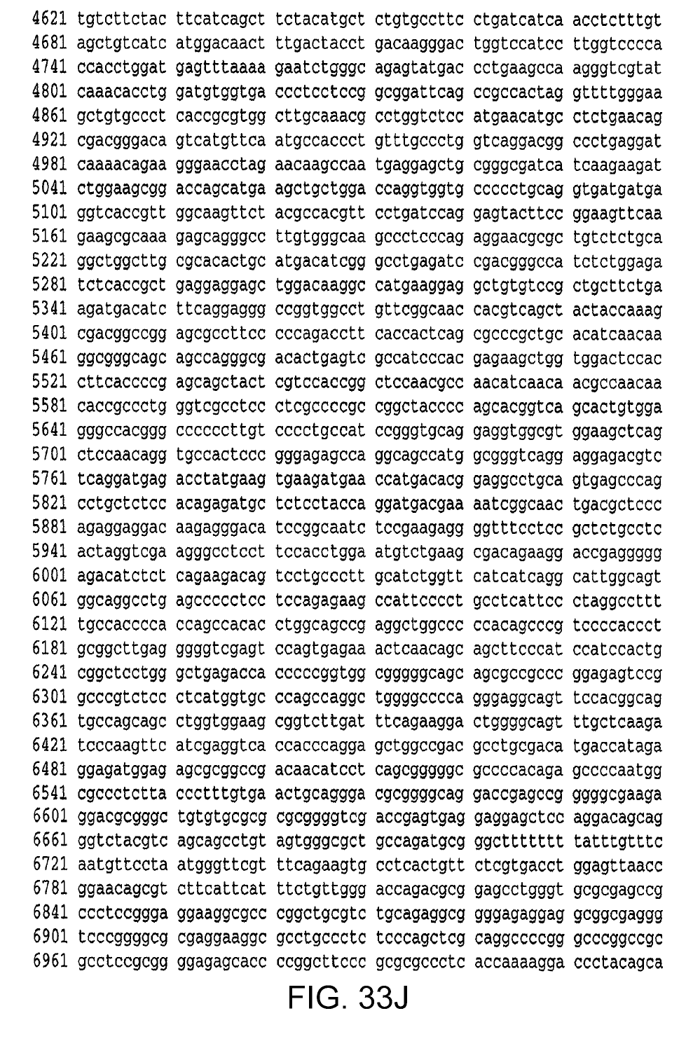

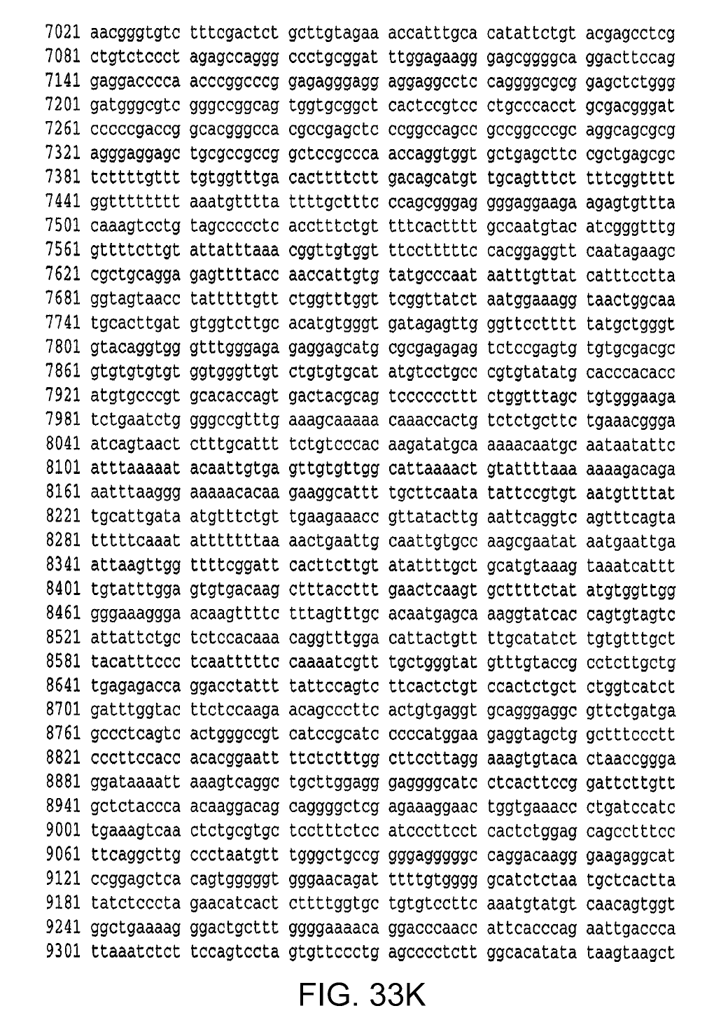

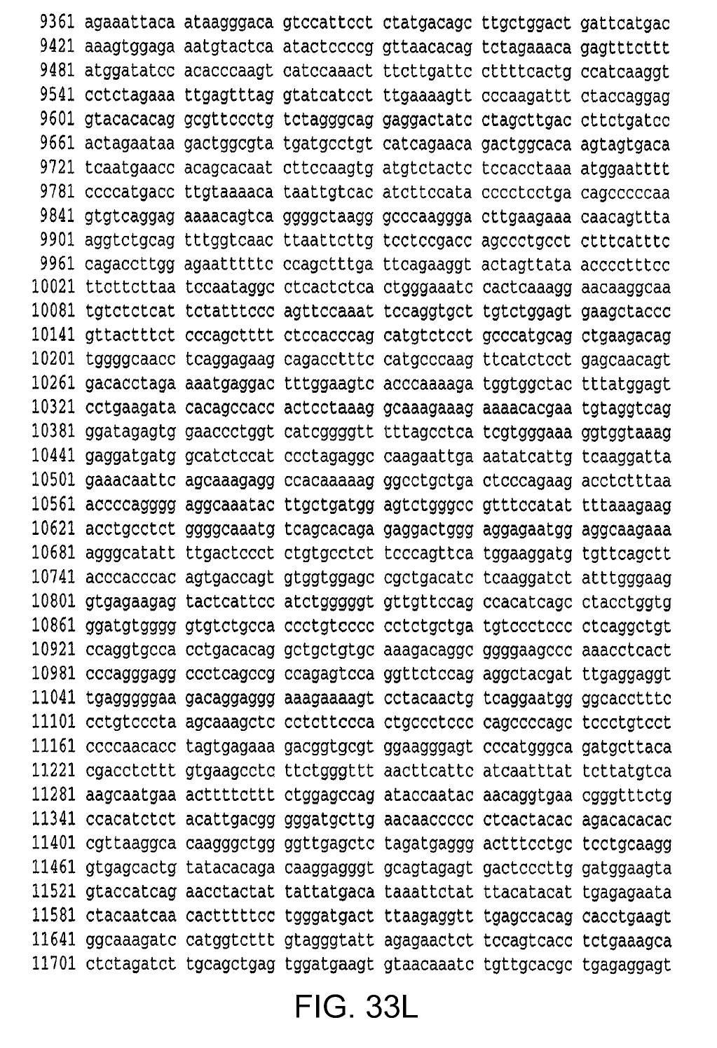

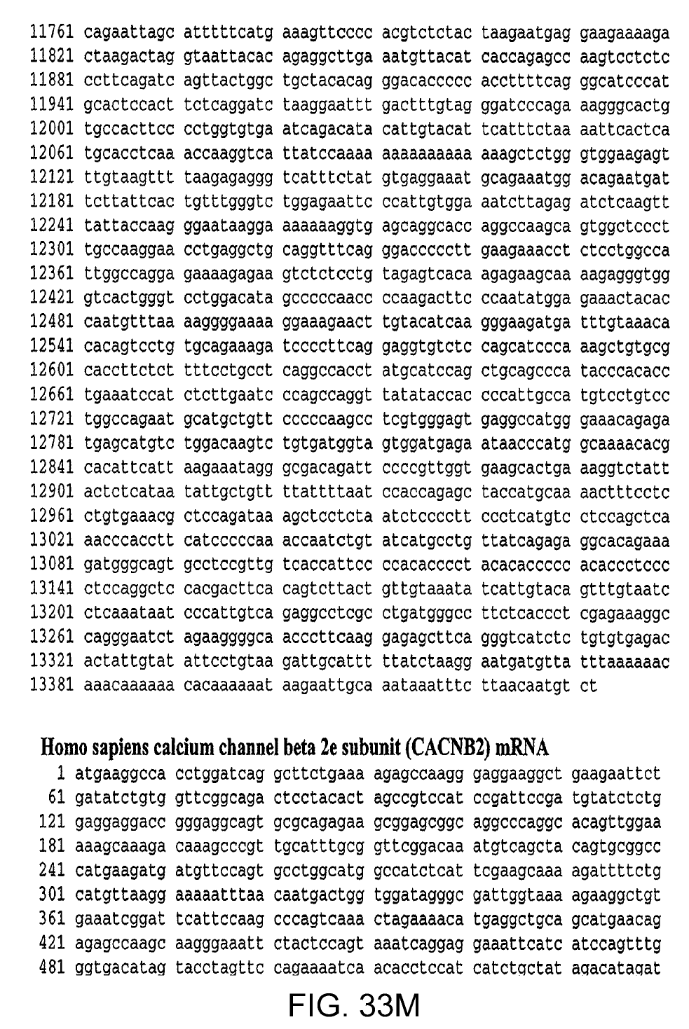

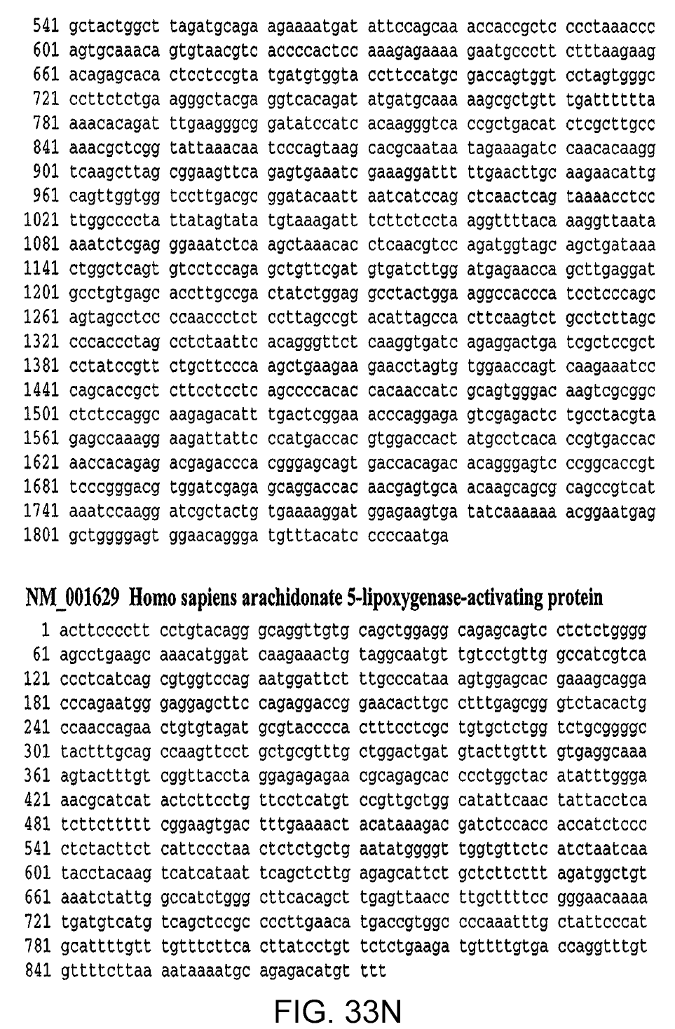

The invention features compositions and methods that are useful for the individualized selection of antihypertensive agents, therapeutic regimens and patient risk assessment. The invention is based, at least in part, on the discovery of correlations between cardiovascular outcomes and genotype for individuals having specific sequence variations. This information can be used for the selection of particular therapeutic or prophylactic agents or treatment regimens by genotype. Specifically, genetic information related to variations in nucleic acid sequence in the α-adducin (ADD1) gene, calcium activated potassium channel (KCNMB1) gene, Beta1-adrenergic receptor (ADRB1) gene, Beta2-adrenergic receptor (ADRB2) gene, the leukotriene A4 hydrolase (LTA4H) gene, the arachidonate 5-lipoxygenase-activating protein (ALOX5AP), CACNA1C, CACNB2, and ALOX5 can influence therapeutic agent selection and/or risk of an adverse cardiovascular outcome (FIG. 33).

### α-Adducin

The α-adducin (ADD1) gene has emerged as both a potential risk factor for hypertension, as well as candidate for effect modification of antihypertensive therapy (Barlassina et al., J Hypertens. 1997; 15:1567-71; Bianchi et al., Ann N Y Acad Sci. 2003; 986:660-8). Adducin is a ubiquitously expressed heterodimeric cytoskeleton protein that promotes the binding of spectrin with actin and may modulate a variety of other cell functions such as ion transport (Bianchi et al., Ann N Y Acad Sci. 2003; 986:660-8; Matsuoka et al., Cellular and Molecular Life Sciences. 2000; 57:884-895). Animal models established that single nucleotide polymorphisms (SNPs) in the ADD1 gene lead to increased tubular sodium reabsorption and hypertension (Tripodi et al., J Clin Invest. 1996; 97:2815-22; Cusi et al., Kidney Int. 1996; 49:1754-9). In humans, a guanine to thymine single nucleotide polymorphism at nucleotide 614 in exon 10 of the ADD1 gene (rs4961) leads to a glycine (Gly) to tryptophan (Trp) change at amino acid position 460. An association between this polymorphism an elevated untreated blood pressure, hypertension, salt sensitivity, and response to diuretic therapy has been proposed (Lanzani et al., Journal of Hypertension. 2005; 23:543-549; Cusi et al., Lancet. 1997; 349:1353-1357; Grant et al., Hypertension. 2002; 39:191-196; Glorioso et al., Hypertension. 1999; 34:649-654; Barlassina et al., Kidney International. 2000; 57:1083-1090; Sciarrone et al., Hypertension. 2003; 41:398-403); however, as with other candidate genes investigated in multifactorial polygenic diseases, the evidence in support of this association has been inconsistent between studies and across populations (Bianchi et al., American Journal of Hypertension. 2000; 13:739-743; Castejon et al., American Journal of Hypertension. 2003; 16:1018-1024; Ciechanowicz et al., Kidney & Blood Pressure Research. 2001; 24:201-206; Turner et al., American Journal of Hypertension. 2003; 16:834-839; Maitland-van der Zee et al., Pharmacogenet Genomics. 2005; 15:287-93).

The present report overcomes weaknesses present in previous studies, and provides the first evaluation of a large, ethnically diverse cohort of coronary artery disease patients with well controlled blood pressure. Results reported herein indicate that the ADD1 460Trp polymorphism is a strong independent predictor for cardiovascular morbidity and mortality; and that this effect is particularly strong in blacks. Accordingly, methods of the invention identify carriers of the ADD1 460Trp as at high risk for adverse cardiovascular outcomes; and indicate that such patients would benefit from early and aggressive therapeutic intervention to ameliorate this risk. Such therapeutic intervention is particularly advantageous for patients that identify themselves as black given that such patients are at higher risk of adverse outcomes than those of other racial/ethnic backgrounds.

Contrary to previous studies, no differences in the response to diuretic therapy were observed between ADD1 variant carriers and non-carriers. This indicates that ADD1 genotype is not a clinically or economically useful guide in selecting a diuretic as part of antihypertensive therapy.

### Large-Conductance Calcium and Voltage-Dependent Potassium Channel

The large-conductance calcium and voltage-dependent potassium (BK) channel found in vascular smooth muscle is comprised of pore-forming-α and regulatory-β1 subunits. The BK channel, particularly the β1 subunit, functions in a negative feedback mechanism to enhance calcium sensitivity, decrease cell excitability, and limit smooth muscle contraction (Fernandez-Fernandez et al., J Clin Invest 2004; 113(7):1032-9). The gene that encodes the β1 subunit of the BK channel is KCNMB1, which is located on chromosome 5q34. KCNMB1 has two commonly occurring, non-synonymous, single nucleotide polymorphisms (SNPs), Glu65Lys and Val110Leu.

Calcium channel blockers are commonly used in the treatment of hypertension and angina, but the response to such agents is widely variable. For example, a study of hypertensive subjects found systolic blood pressure response to verapamil to range from a 33 mmHg decline to a 4 mmHg increase, with an average 12 mmHg decrease (Nguyen et al., J Clin Pharmacol 2000; 40:1480-7). As reported herein, a genetic component to this variability in response to calcium channel blockers has been identified. Calcium activated potassium channel (KCNMB1) genotype influences responsiveness to verapamil SR and risk of adverse cardiovascular outcomes. Importantly, Lys65 variant carrier status was significantly associated with the need for fewer drugs to achieve BP control. Lys65 variant carriers had the most favorable BP response to verapamil.

In addition, the Leu110 variant was associated with a 33% reduced risk of adverse cardiovascular outcomes among patients with hypertension and coronary artery disease. Methods of the invention identify hypertensive coronary artery disease patients who are Leu110 carriers as benefiting from therapy with a calcium channel blocker. Methods of the invention identify women, individuals less than 70 years, and Black individuals that carry the Leu110 variant as having a lower risk of adverse outcomes than individuals of other genotypes.

### Beta1 and Beta2 Adrenergic Receptor (ADRB1)

The β₁ and β₂ adrenergic receptors are seven-transmembrane Gₛ-protein-coupled receptors. Both the β₁ and β₂ adrenergic receptors are expressed in the cardiovascular system, and more particularly, both receptors are co-expressed in cardiomyocytes. The β₁ and β₂ adrenergic receptor (B1AR and B2AR) genes ADRB1 and ADRB2 have common nonsynonymous coding polymorphisms that have proven to be functionally important. ADRB1 variant 145 A>G (Ser49Gly or S49G, rs1801252) and 1165 C>G (Arg389Gly or R389G, rs1801253) have been shown to have altered agonist-promoted receptor down-regulation and decreased Gs coupling and receptor binding (Small et al., Annu Rev Pharmacol Toxicol. 2003; 43:381-411; Mason et al., J Biol Chem. 1999; 274:12670-12674; Rathz et al., J Cardiovasc Pharmacol. 2002; 39:155-160). These two polymorphisms are in linkage disequilibrium so that the haplotype Gly49Gly389 rarely occurs.

In the coding region of the human ADRB2, variant 46 G>A (Gly16Arg or G16R, rs1042713) and 79 C>G (Gln27Glu or Q27E, rs1042714) have been reported to have effects on agonist-induced receptor downregulation and densensitization (Green et al., Biochemistry. 1994; 33:9414-9419; Liggett et al., N Engl J Med. 2002; 346:536-538; Dishy et al., N Engl J Med. 2001; 345:1030-1035). A common synonymous variant 523 C>A at codon 175 (rs1042718) has been associated with several clinical phenotypes, but its functional basis is not known.

Common polymorphisms within the β1-adrenergic receptor include an alteration at nucleotide 46 A>G that results in a R16G, an alteration at nucleotide 79 C>G that results in a Q27E, and an alteration at nucleotide 523 C>A. Methods of the invention identify carriers of the 46G-79C-523C haplotype as having increased risk of an adverse cardiovascular outcome. Such patients could benefit from early and aggressive therapeutic intervention. Methods of the invention indicate that non-carriers of 46G-79G-523C benefit from atenolol therapy, while 46G-79G-523C homozygotes benefit from verapamil therapy.

β1-AR variants (49S-389R haplotype) may be of prognostic importance in hypertensive patients with coronary artery disease. Methods of the invention identify β1-AR variants (49S-389R haplotype) as at high risk for adverse cardiovascular outcomes and also identify these patients as benefiting from β-blocker therapy.

Beta2-adrenergic receptor (ADRB2) genotype differentially effects cardiovascular outcomes for patients treated with atenolol versus verapamil; leukotriene A4 hydrolase (LTA4H) genotype differentially effects cardiovascular outcomes for those treated with atenolol versus verapamil. ADRB2 variants may also modify the outcomes associated with antihypertensive therapy, such that non-carriers of 46G-79G-523C may have better outcomes with atenolol, while verapamil SR may be preferred in 46G-79G-523C homozygotes.

### Arachidonic Acid 5-Lipoxygenase Pathway

The enzyme 5-lipoxygenase (5-LO) catalyzes the first two reactions in the pathway leading to the formation of leukotrienes from arachidonic acid. Leukotrienes are proinflammatory lipid mediators that are associated with atherosclerosis. Leukotriene A(4) hydrolase (LTA4H; 151570), which is present at gene map locus 12q22, is one of the next enzymes in the arachidonic acid cascade. Recent studies suggest a role for the arachidonic acid 5-lipoxygenase pathway in cardiovascular disease. An association between single nucleotide polymorphisms (SNP) and haplotypes of arachidonate 5-lipoxygenase pathway genes and cardiovascular events was identified as reported in more detail below. The haplotype GTC in arachidonate 5-lipoxygenase-activating protein (ALOX5AP) gene was associated with decreased risk of adverse events in Caucasians; for LTA4H, two SNPs in the LTA4H gene (rs2247570 TT and rs1978331 AA) were positively associated with event risk in African Americans. Thus, methods of the invention identify patients, particularly black patients with cardiac arterial disease, that carry variants in arachidonate 5-lipoxygenase pathway genes as having an increased risk of having an adverse cardiovascular outcome. Methods of the invention identify patients that are LTA4H (rs2660845) AA homozygotes as benefiting from atenolol treatment; patients having the LTA4H G-allele have similar benefits regardless of whether they receive verapamil SR or atenolol treatment.

### Detection of Cardiovascular Disease-Related Allele Variants

The present invention features diagnostic assays that employ genetic information (e.g., sequence information related to α-adducin (ADD1), calcium activated potassium channel (KCNMB1), β1-adrenergic receptor (ADRB1), β2-adrenergic receptor (ADRB2), leukotriene A4 hydrolase (LTA4H), arachidonate 5-lipoxygenase-activating protein (ALOX5AP) CACNA1C, CACNB2, or ALOX5, nucleic acid molecules) in evaluating cardiovascular disease prognosis or therapy selection in a subject. Accordingly, the invention provides assays that assess a subject's risk of an adverse cardiovascular event (e.g., fatal or nonfatal myocardial infarction, stroke, or cardiovascular-related death) or the subject's risk of other pathology (e.g., diabetes) using genetic information. Allele variants or other sequence variations are determined using any method known in the art. In one embodiment, PCR probes that are capable of detecting an alteration in an α-adducin (ADD1), calcium activated potassium channel (KCNMB1), β1-adrenergic receptor (ADRB1), β2-adrenergic receptor (ADRB2), leukotriene A4 hydrolase (LTA4H), arachidonate 5-lipoxygenase-activating protein (ALOX5AP), CACNA1C, CACNB2, ALOX5 nucleic acid molecule, including genomic sequences, or closely related molecules, are used to hybridize to a nucleic acid sequence derived from a subject having a cardiovascular disease or at risk of developing such conditions. The specificity of the probe, whether it is made from a highly specific region, e.g., the 5′ regulatory region, or from a less specific region, e.g., a conserved motif, and the stringency of the hybridization or amplification (maximal, high, intermediate, or low), determines whether the probe hybridizes to a naturally occurring sequence, allelic variants, or other related sequences. Hybridization techniques may be used to identify mutations indicative of a cardiovascular disease or adverse clinical outcome in CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) nucleic acid molecule. In yet another embodiment, humans may be diagnosed for a propensity to develop a cardiovascular disease or adverse clinical outcome by direct analysis of the sequence of CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) nucleic acid molecule.

In a preferred embodiment, CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) allele variants are detected in a subject with a history of cardiovascular disease and compared to corresponding nucleic acid sequences present in a reference sample. Reference samples preferably include samples taken from subjects with no personal or family history of cardiovascular disease. Alterations in the sequence of a CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) nucleic acid as compared to the reference sample can be used to diagnose a cardiovascular condition or to predict a propensity to develop a cardiovascular condition. Alterations in the nucleic acid sequence of CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) as compared to a reference sequence can also be used to predict the subject's risk of an adverse cardiovascular event, or to select a therapeutic or prophylactic agent or treatment regimen.

The present invention provides methods of selecting therapeutic or prophylactic agents or treatment regimens and subsequently treating cardiovascular disease and/or disorders or symptoms thereof. In one embodiment, the methods comprise administering a therapeutically effective amount of a pharmaceutical composition comprising a compound of the formulae herein (e.g., verapamil SR, atenolol) to a subject (e.g., a mammal such as a human). Thus, one embodiment is a method of treating a subject suffering from or susceptible to a cardiovascular disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) allele variants may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with cardiovascular disease, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Medical History Metrics

In addition to use of the genetic analysis disclosed herein, the present invention makes use of additional factors in gauging an individual's risk for developing a cardiovascular disease. In particular, one could consider factors, including, for example, age, race or ethnic group, sex, body mass index, blood pressure, smoking status, alcohol consumption history, smoking history, exercise history, diet, family history of cardiovascular disease, low density lipid (LDL) or high density lipid (HDL) cholesterol level, systolic blood pressure, dyastolic blood pressure, history of heart failure, diabetes, renal insufficiency, left ventricular hypertrophy, or combinations thereof to improve the predictive accuracy of conventional methods. A history of cardiovascular disease, myocardial infarction, or stroke in a relative and the age—at which the relative was diagnosed with cardiovascular disease or suffered the myocardial infarction or stroke are also important personal history factors. The inclusion of personal history measures with genetic data in an analysis to predict a phenotype, such as a cardiovascular disease, adverse cardiovascular outcome, or selection of therapeutic agent or treatment regimen, is grounded in the observation that almost all phenotypes are derived from a dynamic interaction between an individual's genes and the environment in which these genes act.

The invention employs metrics (e.g., mathematical methods) for evaluating whether a relationship exists between genetic information and risk of an adverse cardiovascular outcome. The predictive accuracy of such methods is generally improved when the effect of one or more other factors on cardiovascular prognosis is considered. A metric may be used, for example, to correlate a cardiovascular disease or the propensity to develop a cardiovascular disease with a variant allele of a nucleic acid molecule of interest, alone or in combination with other factors. In one embodiment, a metric (e.g., an algorithm or mathematical formula) is used to determine whether a correlation exists between the presence of an allele variant and a cardiovascular disease or adverse cardiovascular event. Metrics are generally used to identify a positive or negative relationship between an allelic variant and a diagnosis, prognosis, or risk of adverse outcome.

Standard methods are used to detect an allele variant in any bodily fluid or tissue sample derived from a subject. Bodily fluids, include, but are not limited to, urine, serum, plasma, saliva, and blood. Tissue samples include, but are not limited to, buccal swabs, washings, or scrapings. An allele variant present in particular nucleic acid molecules or polypeptides may be correlated with a cardiovascular disease or adverse cardiovascular event. Such allele variants are useful in determining a diagnosis or prognosis of a subject. Oligonucleotides or longer fragments derived from CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) nucleic acid sequence may be used as a probe to identify subjects having a genetic variation, mutation, or polymorphism (e.g., restriction fragment length polymorphism (RFLP) or single nucleotide polymorphism (SNP)) in CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) nucleic acid molecule. Such mutations are indicative of a predisposition to develop a cardiovascular condition, an adverse cardiovascular outcome, risk of other pathology (e.g., diabetes) or to select a therapeutic regimen for a subject. Such polymorphisms may affect nucleic acid or polypeptide expression levels or biological activity.

Detection of genetic variation, mutation, or polymorphism relative to a normal, reference sample (e.g., wild-type nucleic acid sequence) can be used as a diagnostic indicator of cardiovascular disease or adverse outcome, the propensity to develop a cardiovascular disease, or may be helpful in selecting a therapeutic agent or treatment regimen for a subject. Genetic alterations may be present in the promoter sequence, open reading frame, intronic sequence, or untranslated 3′ region of CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) gene. Information related to genetic alterations can be used to diagnose a subject as having a cardiovascular disease, or a propensity to develop such a condition or other pathology. As noted throughout, specific alterations in CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) gene can be correlated with the likelihood of developing a cardiovascular disease or adverse cardiovascular outcome, or the predisposition or propensity to develop the same.

One skilled in the art, having detected a given mutation, can then assay one or more factors (e.g., age, race, sex, body mass index, blood pressure, smoking status, low density lipid (LDL) or high density lipid (HDL) cholesterol level, systolic blood pressure, dyastolic blood pressure, history of heart failure, diabetes, renal insufficiency, or left ventricular hypertrophy, alcohol consumption history, smoking history, exercise history, diet, and family history of cardiovascular disease). Each of these factors constitutes a cardiovascular disease risk factor. The presence of such factors together with the presence or absence of an allelic variant is then correlated with an increase or decrease in the likelihood that the subject has or will have a cardiovascular disease, adverse cardiovascular event, or to select a therapeutic agent or treatment regimen most likely to result in a beneficial outcome for the subject.

### Treatment Selection

Analyzing a subject's genotype at one or more genetic loci that are correlated with drug responsiveness provides for individualized treatment regimens. Assaying the subject's genotype prior to administering a therapeutic streamlines the selection of effective therapeutic agents. The present invention provides for the identification of subjects with difficult-to-control hypertension or those susceptible to adverse cardiovascular outcomes. Early genetic identification allows more aggressive therapies to be administered at the time that treatment is initiated. In addition, it reduces polypharmacy in patient's whose cardiovascular condition is ameliorated by monotherapy.

In one embodiment, the presence or absence of an allele variant is used to identify a cardiovascular disease that is responsive to treatment with a particular therapeutic agent (e.g., verapamil or atenolol). In other embodiments, methods for assaying the risk of an adverse cardiovascular outcome are useful in selecting an appropriate cardiovascular therapy. Where the risk of an adverse cardiovascular outcome is high, the methods of the invention indicate the need for early and aggressive cardiovascular therapy. An early and/or aggressive cardiovascular risk reduction management strategy is characterized by the use of two or more agents for the control of hypertension, along with aggressive management of other cardiovascular risk factors (e.g. aggressive lowering of cholesterol, aggressive glucose control in diabetics, smoking cessation, exercise for all and weight loss in those overweight or obese).

Therapeutic agents for the treatment of cardiovascular conditions are known in the art. Treatment regimens for heart failure may include any one or more of ACE inhibitors, such as captopril (Capoten®), enalapril (Vasotec®), ramipril (Altace®), lisinopril (Prinivil®, Zestril®), quinapril (Accupril®), fosinopril (Monopril®), benazepril (Lotensin®), moexipril (Univasc®), trandolapril, perindopril; diuretics, such as hydrochlorothiazide (HydroDIURIL®), chlorothiazide (Diuril®), furosemide (Lasix®), bumetanide (Bumex®), spironolactone (Aldactone®), triamterene (Dyrenium®), metolazone (Zaroxolyn®), torsemide, indapamide, polythiazide, amiloride, combination agents (Dyazide®); vasodilators, such as isosorbide dinitrate (Isordil®), nesiritide (Natrecor®), hydralazine (Apresoline®), nitrates, minoxidil; digitalis glycosides; angiotensin receptor blockers (ARBs), such as losartan and candesartan; and β-blockers, including carvedilol (Coreg®), metoprolol (Lopressor®, Toprol XL®), atenolol, bisoprolol, labetalol, propranolol, sotalol, pindolol, penbutolol, acebutolol, timolol, nadolol, betaxolol; anti-coagulants, including warfarin (Coumadin®), and heparin; angiotensin II receptor blockers including losartan (Cozaar®), valsartan (Diovan®), irbesartan (Avapro®), candesartan, eprosartan, telmisartan, olmesartan; Treatment regimens for hypertension include any one or more of calcium channel blockers, Amlodipine (Norvasc), Diltiazem (Cardizem, Dilacor XR), Nifedipine (Adalat, Procardia).

Treatment regimens for hypertension and other cardiovascular conditions may include any one or more of diuretics, including amiloride (Midamor), bumetanide (Bumex), chlorothiazide (Diuril), chlorthalidone (Hygroton), furosemide (Lasix), hydrochlorothiazide (Esidrix, Hydrodiuril), indapamide (Lozol), spironolactone (Aldactone); ACE inhibitors including Benazepril (Lotensin), Captopril (Capoten), Enalapril (Vasotec), Fosinopril (Monopril), Lisinopril (Prinivil, Zestril), Moexipril (Univasc), Perindopril (Aceon), Quinapril (Accupril), Ramipril (Altace), and Trandolapril (Mavik); Angiotensin-2 Receptor Antagonists Candesartan (Atacand), Eprosartan (Teveten), Irbesartan (Avapro), Losartan (Cozaar), Telmisartan (Micardis), Valsartan (Diovan); Beta blockers including Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (Inderal), Sotalol (Betapace), Timolol (Blocadren); Calcium channel blockers including Amlodipine (Norvasc, Lotrel), Bepridil (Vascor), Diltiazem (Cardizem, Tiazac), Felodipine (Plendil), Nifedipine (Adalat, Procardia), Nimodipine (Nimotop), Nisoldipine (Sular), Verapamil (Calan, Isoptin, Verelan); Alpha Blockers including Doxazosin mesylate (Cardura), Prazosin hydrochloride (Minipress), Prazosin and polythiazide (Minizide), Terazosin hydrochloride (Hytrin); Central Alpha Agonists including Clonidine hydrochloride (Catapres), Clonidine hydrochloride and chlorthalidone (Clorpres, Combipres), Guanabenz Acetate (Wytensin), Guanfacine hydrochloride (Tenex), Methyldopa (Aldomet), Methyldopa and chlorothiazide (Aldoclor), and hydrochlorothiazide (Aldoril).

### Prophylaxis

The invention provides for the identification of candidates for prophylactic cardiovascular treatment. Where a subject is identified as having an allelic variant that increases the risk of cardiovascular disease or an adverse cardiovascular event, the invention further provides for the selection of prophylactic or therapeutic methods. Those assessed as at high risk of developing an adverse outcome of cardiovascular disease are considered as those who might benefit from prophylaxis. Obesity is one risk factor that is associated with an increase in cardiovascular disease. Obesity is increasingly prevalent, both in the U.S. and in the developing world. It is estimated that 33% of the US population is obese (Kuczmarski et al., JAMA. 1994; 272:205-211). Obesity is associated with a number of comorbidities, including heart disease and diabetes. Diets high in fat and calories and reduced physical activity likely contribute to the increase in obesity, heart disease and diabetes. One measure of obesity is body mass index (BMI), which is defined as weight in kilograms divided by height in meters squared (kg/m²). A BMI <21 appears to be associated with protection from coronary heart disease mortality. However, a BMI <25 is generally considered healthy. Subjects having a BMI >25 to 30 are at increased risk for coronary atherosclerosis and diabetes. Subjects having a BMI >30 are considered obese. Obesity is associated with increases in insulin resistance, hyperglycemia, high blood pressure, blood cholesterol and triglyceride levels, and reduced levels of high density lipoproteins (HDLs).

Diabetes is an independent risk factor for cardiovascular disease (Wilson, Circulation. 1998; 97:1837-1847). In assessing a diabetic or obese subject's risk for an adverse outcome associated with cardiovascular disease (e.g., myocardial infarction or stroke) other contributing risk factors include smoking, elevated blood pressure, abnormal serum lipids and lipoproteins, and hyperglycemia, excess body weight and abdominal obesity, physical inactivity, and genetic predisposition to cardiovascular disease. Subjects having a genetic propensity to develop cardiovascular disease and one or more risk factors are likely to benefit from prophylaxis. Prophylaxis includes the use of medications to reduce hypertension; the use of statins to reduce cholesterol; the use of sulfonylurea, metformin, or insulin to control hyperglycemia, the use of behavioral or therapeutic methods to control weight or create a beneficial change in the body mass index, to reduce smoking or alcohol consumption, or to increase exercise.

### Methods for Monitoring Cardiovascular Condition

Any of the diagnostic or prognostic methods and metrics described above can be used to monitor a subject with a history of cardiovascular disease or to diagnose a cardiovascular condition or to predict a propensity to develop an adverse cardiovascular event. A subject having an allelic variant is subject to cardiovascular monitoring that is performed on a regular basis (e.g., once a month, once every six months, yearly, every other year, or less frequently) to assist in the diagnosis, prediction, or prevention of future cardiovascular adverse events or conditions. Such monitoring includes, but is not limited to, assaying systolic or diastolic blood pressure, monitoring blood cholesterol level (low density or high density) or blood glucose, body mass index, weight gain or loss, smoking cessation, alcohol consumption, body mass index, cardiac function, or left ventricular function.

The diagnostic methods described herein can be used individually or in combination with any other diagnostic method described herein for a more accurate assessment of the presence of, severity of, or estimated time of onset of cardiovascular disease or adverse cardiovascular event of a cardiovascular condition. In addition, the diagnostic methods described herein can be used in combination with any other diagnostic methods determined to be useful for the accurate diagnosis of the presence of, severity of, or estimated time of onset of a cardiovascular disease.

In one embodiment, the diagnostic methods described herein can be used in combination with methods designed to monitor and/or manage a cardiovascular disease in a subject. In one example, if a subject is determined to have CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) allele variant, then the methods of the invention are combined with methods of measuring monitoring blood cholesterol level (low density or high density), body mass index, weight gain or loss, smoking cessation, alcohol consumption, body mass index, cardiac function, or left ventricular function, or other prognostic, diagnostic, or clinical parameter. In this embodiment, the diagnostic levels of any or all of these factors, are measured repeatedly as a method of not only diagnosing disease but also monitoring the treatment and management of the cardiovascular disease or condition. If desired, the monitoring is conducted prior to, during, or following treatment with a therapeutic agent, such as verapamil SR or atenolol. Such monitoring may be useful, for example, in assessing the efficacy of a particular drug in a subject or in assessing disease progression. If desired, treatment efficacy or disease progression is correlated with the subject's genotype.

### Diagnostic Methods and Treatment Selection

The diagnostic methods of the invention can be used, for example, with patients that have a disease or condition associated with a cardiovascular disease, in an effort to facilitate selection of an appropriate course of treatment. The diagnostic methods of the invention can also be used with patients who have not yet developed, but who are at risk of developing, a cardiovascular disease or condition, or with patients that are at an early stage of developing such a disease or condition. The methods of the invention can be used to diagnose (or to prevent or treat) the disorders described herein in any mammal, for example, in humans, domestic pets, or livestock.

A mutation in a CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) gene can be detected in a subject in any tissue where the cell contains a nucleus, even one in which this protein is not expressed in that cell or tissue. It may be preferable to detect genes variations in other, more easily obtained sample types, such as in blood or biological fluid samples. Detection of a mutation in a gene encoding a CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H) protein, or arachidonate 5-lipoxygenase-activating protein (ALOX5AP) can be carried out using any standard diagnostic technique. For example, a biological sample obtained from a patient can be analyzed for one or more mutations in nucleic acid molecules encoding a CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) using a mismatch detection approach. Generally, this approach involves polymerase chain reaction (PCR) amplification of nucleic acid molecules from a patient sample, followed by identification of a mutation (i.e., a mismatch) by detection of altered hybridization, aberrant electrophoretic gel migration, binding, or cleavage mediated by mismatch binding proteins, or by direct nucleic acid molecule sequencing. Any of these techniques can be used to facilitate detection of a mutant gene encoding a CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H) or arachidonate 5-lipoxygenase-activating protein (ALOX5AP) protein, and each is well known in the art. For instance, examples of these techniques are described by Orita et al. (Proc. Natl. Acad. Sci. U.S.A. 86:2766-2770, 1989) and Sheffield et al. (Proc. Natl. Acad. Sci. U.S.A. 86:232-236, 1989).

As noted above, in addition to facilitating diagnosis of an existing disease or condition, mutation detection assays also provide an opportunity to diagnose a predisposition to disease related to a mutation in a CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H) or arachidonate 5-lipoxygenase-activating protein (ALOX5AP) gene before the onset of symptoms. For example, a patient who is heterozygous for a gene encoding an abnormal CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) protein (or an abnormal amount thereof) that suppresses normal CACNA1C, CACNB2, ALOX5, α-adducin (ADD1), calcium activated potassium channel (KCNMB1), β1-adrenergic receptor (ADRB1), β2-adrenergic receptor (ADRB2), or leukotriene A4 hydrolase (LTA4H) biological activity or expression may show no clinical symptoms of a disease related to such proteins, and yet possess a higher than normal probability of developing such disease. Given such a diagnosis, a patient can take precautions to minimize exposure to adverse environmental factors, and can carefully monitor their medical condition, for example, through frequent physical examinations, by losing weight, reducing alcohol consumption or smoking, or increasing exercise.

While it may be preferable to carry out diagnostic methods for detecting a mutation in a CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) gene using genomic DNA from readily accessible tissues or fluids, as noted above, genomic DNA, mRNA encoding this protein, or the protein itself, can also be assayed from tissue samples in which it is expressed. The sequence or expression levels of a gene encoding CACNA1C, CACNB2, ALOX5, α-adducin (ADD1), calcium activated potassium channel (KCNMB1), β1-adrenergic receptor (ADRB1), β2-adrenergic receptor (ADRB2), leukotriene A4 hydrolase (LTA4H), or arachidonate 5-lipoxygenase-activating protein (ALOX5AP) can be assayed, for example, in an oral tissue sample or a biological fluid from a patient. The presence of a mutation that results in an alteration in gene expression can be determined by using any of a number of standard techniques that are well known in the art, including northern blot analysis and quantitative PCR (see, e.g., Ausubel et al., supra; PCR Technology: Principles and Applications for DNA Amplification, H. A. Ehrlich, Ed., Stockton Press, NY; Yap et al. Nucl. Acids. Res. 19:4294, 1991).

Once a biologic sample from a subject has been obtained (e.g., a bodily fluid, such as urine, saliva, plasma, serum, or a tissue sample, such as buccal tissue sample or buccal cell) detection of a sequence variation or allelic variant is typically undertaken. Virtually any method known to the skilled artisan is employed. Perhaps the most direct method is to actually determine the sequence of either genomic DNA or cDNA and compare these sequences to the known alleles of the gene. This can be a fairly expensive and time-consuming process. Nevertheless, this technology is used by numerous bioinformatics companies with interests in single nucleotide polymorphisms (SNPs) including Celera, Curagen, Incyte, Variagenics and Genaissance. Commercially available methods exist for the high volume low cost sequencing of multiple genetic samples. A variation on the direct sequence determination method is the Gene Chip™ method available from Affymatrix. Alternatively, robust and less expensive ways of detecting DNA sequence variation are also commercially available. For example, Perkin Elmer adapted its TAQman Assay™ to detect sequence variation. Orchid BioSciences has a method called SNP-IT™. (SNP-Identification Technology) that uses primer extension with labeled nucleotide analogs to determine which nucleotide occurs at the position immediately 3′ of an oligonucleotide probe. the extended base is then identified using direct fluorescence, indirect colorimetric assay, mass spectrometry, or fluorescence polarization. Sequenom uses a hybridization capture technology plus MALDI-TOF (Matrix Assisted Laser Desorption/Ionization—Time-of-Flight mass spectrometry) to detect SNP genotypes with their MassARRAY™ system. Promega provides the READIT™ SNP/Genotyping System (U.S. Pat. No. 6,159,693). In this method, DNA or RNA probes are hybridized to target nucleic acid sequences. Probes that are complementary to the target sequence at each base are depolymerized with a proprietary mixture of enzymes, while probes which differ from the target at the interrogation position remain intact. The method uses pyrophosphorylation chemistry in combination with luciferase detection to provide a highly sensitive and adaptable SNP scoring system. Third Wave Technologies has the Invader OS™ method that uses a proprietary Cleavaseg enzymes, which recognize and cut only the specific structure formed during the Invader process. Invader OS relies on linear amplification of the signal generated by the Invader process, rather than on exponential amplification of the target. The Invader OS assay does not utilize PCR in any part of the assay. In addition, there are a number of forensic DNA testing labs and many research labs that use gene-specific PCR, followed by restriction endonuclease digestion and gel electrophoresis (or other size separation technology) to detect restriction fragment length polymorphisms (RFLPs). Any of a variety of methods exist for detecting sequence variations may be used in the methods of the invention. The particular method used is not important in the estimation of cardiovascular risk or treatment selection. The key in risk determination and treatment selection is the identification of particular allelic variants and the correlation of these sequence variations with treatment selection, therapy benefit, or risk of an adverse cardiovascular event. This genetic information is useful in isolation, but may be even more useful when coupled with other factors of clinical significance to cardiovascular health. Such factors include age, race, sex, body mass index, blood pressure, smoking status, alcohol consumption history, smoking history, exercise history, diet, family history of cardiovascular disease, low density lipid (LDL) or high density lipid (HDL) cholesterol level, systolic blood pressure, dyastolic blood pressure, history of heart failure, diabetes, renal insufficiency, or left ventricular hypertrophy. The invention employs metrics (e.g., mathematical methods) for evaluating whether a relationship exists between genetic information and risk of an adverse cardiovascular outcome. The predictive accuracy of such methods is generally improved when the effect of one or more other factors on cardiovascular prognosis is considered. A metric may be used, for example, to correlate a cardiovascular disease or the propensity to develop a cardiovascular disease with a variant allele of a nucleic acid molecule of interest, alone or in combination with other factors. In one embodiment, a metric (e.g., an algorithm or mathematical formula) is used to determine whether a correlation exists between the presence of an allele variant and a cardiovascular disease or adverse cardiovascular event.

Polynucleotide amplifications are typically template-dependent. Such amplifications generally rely on the existence of a template strand to make additional copies of the template. Primers are short nucleic acids that are capable of priming the synthesis of a nascent nucleic acid in a template-dependent process, which hybridize to the template strand. Typically, primers are from ten to thirty base pairs in length, but longer sequences can be employed. Primers may be provided in double-stranded and/or single-stranded form, although the single-stranded form generally is preferred.

Often, pairs of primers are designed to selectively hybridize to distinct regions of a template nucleic acid, and are contacted with the template DNA under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

Polymerase Chain Reaction

A number of template dependent processes are available to amplify the oligonucleotide sequences present in a given template sample. One of the best known amplification methods is the polymerase chain reaction. In PCR, pairs of primers that selectively hybridize to nucleic acids are used under conditions that permit selective hybridization. The term primer, as used herein, encompasses any nucleic acid that is capable of priming the synthesis of a nascent nucleic acid in a template-dependent process. Primers may be provided in double-stranded or single-stranded form, although the single-stranded form is preferred. Primers are used in any one of a number of template dependent processes to amplify the target gene sequences present in a given template sample. One of the best known amplification methods is PCR, which is described in detail in U.S. Pat. Nos. 4,683,195, 4,683,202 and 4,800,159, each incorporated herein by reference.

In PCR, two primer sequences are prepared which are complementary to regions on opposite complementary strands of the target-gene(s) sequence. The primers will hybridize to form a nucleic-acid:primer complex if the target-gene(s) sequence is present in a sample. An excess of deoxyribonucleoside triphosphates is added to a reaction mixture along with a DNA polymerase, e.g., Taq polymerase, that facilitates template-dependent nucleic acid synthesis. If the target-gene(s) sequence:primer complex has been formed, the polymerase will cause the primers to be extended along the target-gene(s) sequence by adding on nucleotides. By raising and lowering the temperature of the reaction mixture, the extended primers will dissociate from the target-gene(s) to form reaction products, excess primers will bind to the target-gene(s) and to the reaction products and the process is repeated. These multiple rounds of amplification, referred to as “cycles”, are conducted until a sufficient amount of amplification product is produced.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641, filed Dec. 21, 1990.

LCR

Another method for amplification is the ligase chain reaction (“LCR”). LCR differs from PCR because it amplifies the probe molecule rather than producing amplicon through polymerization of nucleotides. In LCR, two complementary probe pairs are prepared, and in the presence of a target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750, incorporated herein by reference, describes a method similar to LCR for binding probe pairs to a target sequence.

Isothermal Amplification

An isothermal amplification method, in which restriction endonucleases and ligases are used to achieve the amplification of target molecules that contain nucleotide 5′-[α-thio]-triphosphates in one strand of a restriction site also may be useful in the amplification of nucleic acids in the present invention. In one embodiment, loop-mediated isothermal amplification (LAMP) method is used for single nucleotide polymorphism (SNP) typing.

Strand Displacement Amplification

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection.

Transcription-Based Amplification

Other nucleic acid amplification procedures include transcription-based amplification systems, including nucleic acid sequence based amplification. In nucleic acid sequence based amplification, the nucleic acids are prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer, which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Other Amplification Methods

Other amplification methods may be used in accordance with the present invention. In one embodiment, “modified” primers are used in a PCR-like, template and enzyme dependent synthesis. The primers may be modified by labeling with a capture moiety (e.g., biotin) and/or a detector moiety (e.g., enzyme). In the presence of a target sequence, the probe binds and is cleaved catalytically. After cleavage, the target sequence is released intact to be bound by excess probe. Cleavage of the labeled probe signals the presence of the target sequence.

In another approach, a nucleic acid amplification process involves cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Methods for Nucleic Acid Separation

It may be desirable to separate nucleic acid products from other materials, such as template and excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid. Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC. In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized with light exhibiting the appropriate excitatory spectra.

As described above, the specific methods or compositions used to detect polymorphisms (e.g., SNPS, RFLPs) are not significant. The invention employs virtually any method for detecting an allele variation that is known in the art.

### Microarrays

In another approach, the nucleic acid molecules of the invention, or fragments thereof, are useful as hybridizable array elements in a microarray for the detection of sequence alterations, including allele variants in CACNA1C, CACNB2, ALOX5, α-adducin (ADD1) gene, calcium activated potassium channel (KCNMB1) gene, β1-adrenergic receptor (ADRB1) gene, β2-adrenergic receptor (ADRB2) gene, and leukotriene A4 hydrolase (LTA4H) gene. In one preferred embodiment, such arrays include not only wild-type sequences, but also allele variants. The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference.

To produce a nucleic acid microarray oligonucleotides may be synthesized or bound to the surface of a substrate using a chemical coupling procedure and an ink jet application apparatus, as described in PCT application WO95/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, preferably as a bodily fluid (such as blood, saliva, or urine) or tissue sample (e.g. a buccal tissue sample obtained by swabbing, scraping, or washing). For some applications, cultured cells or other tissue preparations may be used. The mRNA is isolated according to standard methods, and cDNA is produced and used as a template to make complementary RNA suitable for hybridization. Such methods are described herein. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. Accordingly, the microarrays of the invention are useful for identifying the presence or absence of allele variants based on hybridization characteristics. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct sequences simultaneously (e.g., Heller et al., Proc. Natl. Acad. Sci. 94:2150-2155, 1997). Preferably, a scanner is used to determine the levels and patterns of fluorescence.

In other approaches, abnormalities of CACNA1C, CACNB2, ALOX5, α-adducin (ADD1) gene, calcium activated potassium channel (KCNMB1) gene, β1-adrenergic receptor (ADRB1) gene, β2-adrenergic receptor (ADRB2) gene, and leukotriene A4 hydrolase (LTA4H) gene that can be detected using the diagnostic methods of the invention include those characterized by, for example, (i) a gene encoding CACNA1C, CACNB2, ALOX5, α-adducin (ADD1), calcium activated potassium channel (KCNMB1), β1-adrenergic receptor (ADRB1), β2-adrenergic receptor (ADRB2), or leukotriene A4 hydrolase (LTA4H) gene containing a mutation that results in the production of an abnormal protein, (ii) an abnormal CACNA1C, CACNB2, ALOX5, α-adducin (ADD1), calcium activated potassium channel (KCNMB1), β1-adrenergic receptor (ADRB1), β2-adrenergic receptor (ADRB2), or leukotriene A4 hydrolase (LTA4H) polypeptide itself (e.g., a mutant protein), and (iii) a mutation in CACNA1C, CACNB2, ALOX5, α-adducin (ADD1), calcium activated potassium channel (KCNMB1), β1-adrenergic receptor (ADRB1), β2-adrenergic receptor (ADRB2), or leukotriene A4 hydrolase (LTA4H) gene that results in production of an abnormal amount of the protein. Detection of such abnormalities can be used to diagnose human diseases or conditions related to CACNA1C, CACNB2, ALOX5, α-adducin (ADD1), calcium activated potassium channel (KCNMB1), β1-adrenergic receptor (ADRB1), β2-adrenergic receptor (ADRB2), or leukotriene A4 hydrolase (LTA4H), such as those relating to a cardiovascular disease.

### Kits or Pharmaceutical Systems

The present compositions may be assembled into kits or pharmaceutical systems for use in diagnosing a cardiovascular condition, adverse cardiovascular outcome, or selecting a therapeutic agent or treatment regimen. In one embodiment, the kit provides agents that detect an allelic variant in CACNA1C, CACNB2, ALOX5, α-adducin (ADD1), calcium activated potassium channel (KCNMB1), 13′-adrenergic receptor (ADRB1), β2-adrenergic receptor (ADRB2), or leukotriene A4 hydrolase (LTA4H) gene. Such agents include probes that hybridize to a CACNA1C, CACNB2, ALOX5, α-adducin (ADD1), calcium activated potassium channel (KCNMB1), β1-adrenergic receptor (ADRB1), β2-adrenergic receptor (ADRB2), or leukotriene A4 hydrolase (LTA4H) allelic variant. In other embodiments, the kit provides primers that differentially amplify CACNA1C, CACNB2, ALOX5, α-adducin (ADD1), calcium activated potassium channel (KCNMB1), 81-adrenergic receptor (ADRB1), β2-adrenergic receptor (ADRB2), or leukotriene A4 hydrolase (LTA4H) allele variant.

Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the agents of the invention. In one example, the kit contains instructions for the use of the kit for the diagnosis of a cardiovascular disease or adverse cardiovascular outcome, or the propensity to develop a cardiovascular disease or adverse cardiovascular outcome. In another example, the kit contains instructions for the diagnosis of cardiovascular conditions or the propensity to develop cardiovascular conditions. In yet another example, the kit contains instructions for the use of the kit to monitor therapeutic treatment or dosage regimens.

### Combination Therapies

Optionally, a cardiovascular disease therapeutic (e.g., verapamil or atenolol) may be administered in combination with any other standard cardiovascular therapy; such methods are known to the skilled artisan and described herein. In particular, patients identified using a method of the invention as having hypertension that is not amenable to treatment using monotherapy, may be treated with one or more of the following agents amiloride (Midamor), bumetanide (Bumex), chlorothiazide (Diuril), chlorthalidone (Hygroton), furosemide (Lasix), hydrochlorothiazide (Esidrix, Hydrodiuril), indapamide (Lozol), spironolactone (Aldactone); ACE inhibitors including Benazepril (Lotensin), Captopril (Capoten), Enalapril (Vasotec), Fosinopril (Monopril), Lisinopril (Prinivil, Zestril), Moexipril (Univasc), Perindopril (Aceon), Quinapril (Accupril), Ramipril (Altace), and Trandolapril (Mavik); Angiotensin-2 Receptor Antagonists Candesartan (Atacand), Eprosartan (Teveten), Irbesartan (Avapro), Losartan (Cozaar), Telmisartan (Micardis), Valsartan (Diovan); Beta blockers including Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (Inderal), Sotalol (Betapace), Timolol (Blocadren); Calcium channel blockers including Amlodipine (Norvasc, Lotrel), Bepridil (Vascor), Diltiazem (Cardizem, Tiazac), Felodipine (Plendil), Nifedipine (Adalat, Procardia), Nimodipine (Nimotop), Nisoldipine (Sular), Verapamil (Calan, Isoptin, Verelan); Alpha Blockers including Doxazosin mesylate (Cardura), Prazosin hydrochloride (Minipress), Prazosin and polythiazide (Minizide), Terazosin hydrochloride (Hytrin); Central Alpha Agonists including Clonidine hydrochloride (Catapres), Clonidine hydrochloride and chlorthalidone (Clorpres, Combipres), Guanabenz Acetate (Wytensin), Guanfacine hydrochloride (Tenex), Methyldopa (Aldomet), Methyldopa and chlorothiazide (Aldoclor), and hydrochlorothiazide (Aldoril).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### ADD1 460Trp Polymorphism is Associated with Cardiovascular Risk

The study included 5,979 patients followed over an average of 2.8 years, was ethnically diverse, and had extensive patient level information including well controlled blood pressure and a rigorous adjudication process for adverse outcomes. In hypertensive coronary artery disease patients ADD1 variant carriers showed a 43% (95% CI 11-84%) excess risk for a primary outcome event. No evidence of effect modification of diuretic therapy, age, or other cardiovascular risk factors was observed. These findings identify a gene that contributes to cardiovascular risk. In view of this finding, the invention provides genetic screens that identify those at high risk for adverse cardiovascular outcomes by identifying variations in ADD1. This is the first study to document a main effect of ADD1 on cardiovascular outcomes, and this main effect was not modified by environmental or clinical factors.

Baseline demographic and study data for the INternational VErapamil SR-trandolapril STudy (INVEST)-GENES cohort by ADD1 460Trp polymorphism are summarized in Tables 1 and 2. Patients in the INVEST-GENES cohort included a large proportion of elderly, diabetic and female patients that were ethnically diverse. The patient cohort included 47% Hispanics, 38% whites and 11% blacks. The overall primary outcome event rate in the INVEST-GENES was 4.6%, and 68.0% of patients used diuretics at some time during the study.

Genotypes for the ADD1 polymorphism were determined for 5,661 (94.7%) of the 5,979 INVEST-GENES patients. These included all patients who experienced a primary outcome event. Primary outcome events are clinically meaningful end-points that specifically referred to non-fatal stroke, non-fatal myocardial infarction (heart attack) and death from all causes. Importantly, patients with missing genotype data show no statistically or clinically significant differences from the 5,979 patients for whom genetic samples were collected (P-values for all patient characteristics listed in Table 1>0.5).

TABLE 1
Baseline Characteristics by ADD1 carrier status*

| | INVEST-GENES | | | | | | |
|---|---|---|---|---|---|---|---|
| | with complete | | | | | | |
| | ADD1 | | ADD1 variant | | Wild type | | |
| | genotypes | | carrier | | homozygotes | | |
| | (n = 5661) | | (n = 1773) | | (n = 3888) | | P† |
| Demographic | | | | | | | |
| Age, mean (SD), y | 66.2 | (9.7) | 66.6 | (9.8) | 66.0 | (9.7) | .04 |
| >70 y | 1931 | (34.1) | 644 | (36.3) | 1287 | (33.1) | .02 |
| Women | 3167 | (55.9) | 984 | (55.5) | 2183 | (56.15) | .65 |
| Ethnicity | | | | | | | |
| White | 2154 | (38.1) | 814 | (45.9) | 1340 | (33.5) | |
| Black | 610 | (10.8) | 99 | (5.6) | 511 | (13.1) | <.001 |
| Hispanic | 2671 | (47.2) | 792 | (44.7) | 1879 | (48.3) | |
| Other | 226 | (4.0) | 68 | (3.8) | 158 | (4.1) | |
| BMI, mean (SD), kg/m² | 29.4 | (5.6) | 29.4 | (5.9) | 29.3 | (5.4) | .61 |
| Condition | | | | | | | |
| Myocardial Infarction | 1319 | (23.3) | 436 | (24.6) | 883 | (22.7) | .12 |
| Angina pectoris | 4207 | (74.3) | 1263 | (71.2) | 2944 | (75.7) | <0.01 |
| CABG ≧ 1 month ago | 808 | (14.3) | 282 | (15.9) | 526 | (13.5) | .02 |
| Stroke/TIA | 388 | (6.9) | 121 | (6.8) | 267 | (6.9) | .95 |
| Left ventricular hypertrophy | 849 | (15.0) | 267 | (15.1) | 582 | (15.0) | .93 |
| Unstable angina ≧1 mo ago | 548 | (9.7) | 184 | (10.4) | 364 | (9.4) | .23 |
| Arrhythmia | 394 | (7.0) | 136 | (7.7) | 258 | (6.6) | .15 |
| Heart failure (class I-III) | 192 | (3.4) | 69 | (3.9) | 123 | (3.2) | .16 |
| Peripheral vascular disease | 627 | (11.1) | 203 | (11.5) | 424 | (10.9) | .54 |
| Smoking | | | | | | | |
| Past | 2339 | (41.3) | 758 | (42.8) | 1581 | (40.7) | .14 |
| within last 30 d | 578 | (10.2) | 180 | (10.2) | 398 | (10.2) | .92 |
| Never | 3322 | (58.7) | 1015 | (57.2) | 2307 | (59.3) | |
| Diabetes | 1344 | (23.7) | 405 | (22.8) | 939 | (24.2) | .28 |
| Hypercholesterolemia | 3087 | (54.5) | 954 | (53.8) | 2133 | (54.9) | .46 |
| Renal impairment | 88 | (1.6) | 26 | (1.5) | 62 | (1.6) | .71 |
| Cancer | 229 | (4.1) | 73 | (4.1) | 156 | (4.0) | .85 |
| Medication | | | | | | | |
| Aspirin/other antiplatelet | 2592 | (45.8) | 857 | (48.3) | 1735 | (44.6) | <.01 |
| therapy | | | | | | | |
| Other NSAIDs | 1341 | (23.7) | 406 | (22.9) | 935 | (24.1) | 0.34 |
| Antidiabetic medication | 1344 | (23.7) | 405 | (22.8) | 939 | (24.2) | 0.28 |
| Any lipid-lowering agent | 2047 | (36.2) | 653 | (36.8) | 1394 | (35.9) | 0.48 |

Abbreviations:
SD, standard deviation;
BMI, body mass index;
CABG, coronary artery bypass graft;
TIA, transient ischemic attack;
BP, blood pressure;
HCTZ, hydrochlorothiazide;
NSAID, non-steroidal anti-inflammatory drug.
*Data are presented as number and percentage unless otherwise indicated;
†T-test for continuous and Chi-squared test for categorical variables

Of the 5,661 patients with genotype data, 1,773 (31.3%) were carriers of the ADD1 variant. ADD1 variant carrier status was more frequent among whites (38%) and less frequent among Hispanics (30%) and Blacks (16%) (Table 3). Baseline characteristics and study drugs of ADD1 variant carriers were generally comparable to those of wild type homozygotes as detailed in Tables 1 and 2. Among Hispanics and Blacks, the ADD1 variant was in Hardy Weinberg Equilibrium, but not among Whites (X²=7.48; P=0.006).

TABLE 2
Baseline Blood Pressure and Antihypertensive Medications,
study drugs and intermediate outcomes

| | ADD1 variant | | Wild type | | |
|---|---|---|---|---|---|
| | carrier | | homozygotes | | |
| | (n = 1773) | | (n = 3888) | | P† |
| Heart rate (SD), beats/min | 74.9 | (9.6) | 74.8 | (9.6) | .96 |
| Blood pressure at study entry, | | | | | |
| mean (SD), mmHg | | | | | |
| Systolic | 148.5 | (18.0) | 147.8 | (18.3) | .18 |
| Diastolic | 85.1 | (10.7) | 85.6 | (10.6) | .12 |
| No. (%) with blood pressure | | | | | |
| in control at study entry | | | | | |
| Systolic | 414 | (26.4) | 949 | (27.4) | .49 |
| Diastolic | 959 | (61.2) | 2008 | (57.9) | .03 |
| Both | 374 | (23.9) | 827 | (23.9) | .98 |
| No. of antihypertensive drugs | 1.7 | (0.8) | 1.6 | (0.8) | .42 |
| at study entry, mean (SD) | | | | | |
| 0 | 205 | (11.6) | 413 | (10.6) | |
| 1 | 813 | (45.9) | 1856 | (47.7) | |
| 2 | 534 | (30.1) | 1136 | (29.2) | .65 |
| 3 | 177 | (10..0) | 379 | (9.7) | |
| >3 | 42 | (2.4) | 96 | (2.5) | |
| Study arm and added drugs | | | | | |
| Verapamil SR-based strategy | 865 | (48.8) | 1901 | (48.9) | .94 |
| HCTZ | 1195 | (67.4) | 2654 | (68.3) | .52 |
| Trandolapril | 1314 | (74.1) | 2885 | (74.2) | .94 |
| No. of strategy drugs at end of | 1.87 | (1.06) | 1.92 | (1.06) | .11 |
| follow-up, mean (SD) | | | | | |
| Total No. of antihypertensive drugs | 2.66 | (1.31) | 2.68 | (1.36) | .64 |
| at end of follow-up, mean (SD) | | | | | |
| Intermediate outcomes | | | | | |
| Percent of visits with blood | 61.0 | (26.5) | 60.5 | (27.2) | .53 |
| pressure in control, mean (SD) | | | | | |
| SBP at 12 months, | 133.2 | (16.1) | 133.4 | (16.5) | .70 |
| mean (SD), mmHg§ | | | | | |
| DBP at 12 months, | 77.7 | (9.0) | 78.1 | (9.6) | .09 |
| mean (SD), mmHg§ | | | | | |
| BP in control at 12 month§, | 1023 | (62.5) | 2175 | (59.8) | .22 |
| No. (%) | | | | | |
| SBP at 24 months, | 131.9 | (15.2) | 131.7 | (15.5) | .68 |
| mean (SD), mmHg§ | | | | | |
| DBP at 24 months, | 77.3 | (8.8) | 77.3 | (9.1) | .92 |
| mean (SD), mmHg§ | | | | | |
| BP in control at 24 months§, | 984 | (65.3) | 2141 | (64.1) | .76 |
| No. (%) | | | | | |

Abbreviations:
SD, standard deviation;
BP, blood pressure;
ACE, angiotensin-converting enzyme;
HCTZ, hydrochlorothiazide;
SBP, systolic blood pressure;
DBP, diastolic blood pressure.
*Data are presented as number and percentage unless otherwise indicated.
†T-test for continuous and Chi-squared test for categorical variables.
‡BP in control as SBP < 140 mmHg and DBP < 90 mmHg
§N = patients at risk at 12 and 24 months, respectively.

TABLE 3
ADD1 Gly460Trp Genotype Frequency for INVEST-GENES
and by Ethnicity

| | Trp/Trp | Gly/Trp | Gly/Gly |
|---|---|---|---|
| INVEST-GENES | 152 (3%) | 1621 (29%) | 3888 (69%) |
| White | 70 (3%) | 744 (35%) | 1340 (62%) |
| Black | 7 (1%) | 92 (15%) | 511 (84%) |
| Hispanic | 64 (2%) | 728 (27%) | 1879 (70%) |
| Other | 11 (5%) | 57 (25%) | 158 (70%) |

### INVEST Treatment Effects

Consistent with the full INVEST study population (Pepine et al., Jama. 2003; 290:2805-16), there was no significant difference in primary outcome events between treatment strategies for patients included in the INVEST-GENES (HR 1.14; 95% CI 0.89-1.46, P=0.30).

Association of ADD1 Carrier Status with Risk of Clinical Outcomes

The incidence of primary outcome and its components by ADD1 variant carrier status, as well as adjusted and unadjusted hazard ratio for variant carrier status are shown in Table 4.

TABLE 4
Adjusted and unadjusted hazard ratios for ADD1 carrier status

| | | | Unadjusted HR | Adjusted HR |
|---|---|---|---|---|
| | Variant | Wild type | (95% CI) for | (95% CI) for |
| | carrier* | homozygotes* | variant carrier | variant carrier |
| | (n = 1773) | (n = 3888) | status | status |
| Primary outcome | 101 (5.7) | 157 (4.0) | 1.42 (1.10-1.82) | 1.43 (1.11-1.84) |
| event | | | | |
| Secondary outcomes | | | | |
| Death | 45 (2.5) | 54 (1.4) | 1.82 (1.23-2.71) | 1.83 (1.22-2.74) |
| CARDIOVASCULAR | 19 (1.1) | 26 (0.7) | 1.60 (0.89-2.89) | 1.59 (0.87-2.90) |
| death | | | | |
| Nonfatal MI | 26 (1.5) | 55 (1.4) | 1.04 (0.65-1.65) | 1.00 (0.63-1.61) |
| Nonfatal stroke | 35 (2.0) | 51 (1.3) | 1.50 (0.98-2.31) | 1.52 (0.98-2.37) |
| Fatal and nonfatal MI | 37 (2.1) | 71 (1.83) | 1.15 (0.77-1.70) | 1.11 (0.74-1.66) |
| Fatal and nonfatal | 37 (2.1) | 54 (1.4) | 1.50 (0.99-2.28) | 1.55 (1.01-2.38) |
| stroke | | | | |

Abbreviations:
HR, hazard ratio;
CI, confidence interval;
CARDIOVASCULAR, cardiovascular;
MI, myocardial infarction.
*number (percentage)

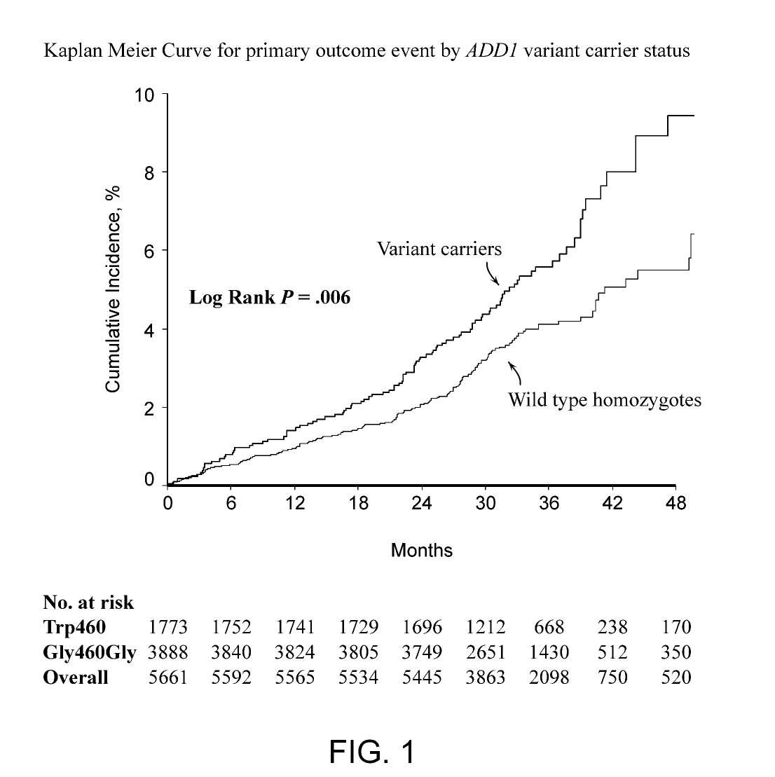

Unadjusted for baseline differences, ADD1 variant carriers had a 42% excess risk for a primary outcome event (P=0.007) and significantly shorter time to primary outcome event than non carriers (Log Rank P=0.006; FIG. 1). After adjusting for prespecified covariates, ADD1 variant carrier status remained associated with significant excess risk for a primary outcome event (HR 1.43, 95% CI 1.11-1.84, P=0.006). Calculated for components of the primary outcome, the adjusted model for ADD1 variant carrier status showed 83% excess risk of all cause death (P=0.003), 55% excess risk of total stroke (P=0.05), a trend toward higher risk for cardiovascular death (59%; P=0.13) and nonfatal stroke (52%; P=0.06) and no difference in the risk of nonfatal myocardial infarction (P=0.99) or total myocardial infarction (P=0.61). For unadjusted hazard ratios see table 4.

### Clinical Outcome Did not Differ Among ADD1 Variant Carriers Using Diuretics

The hypothesis that carriers of one or two copies of the ADD1 460 Trp allele (variant carriers), would be at higher risk of experiencing a primary outcome event and have a greater reduction in risk of a primary outcome event when treated with diuretics compared to ADD1 wild type homozygotes was tested.

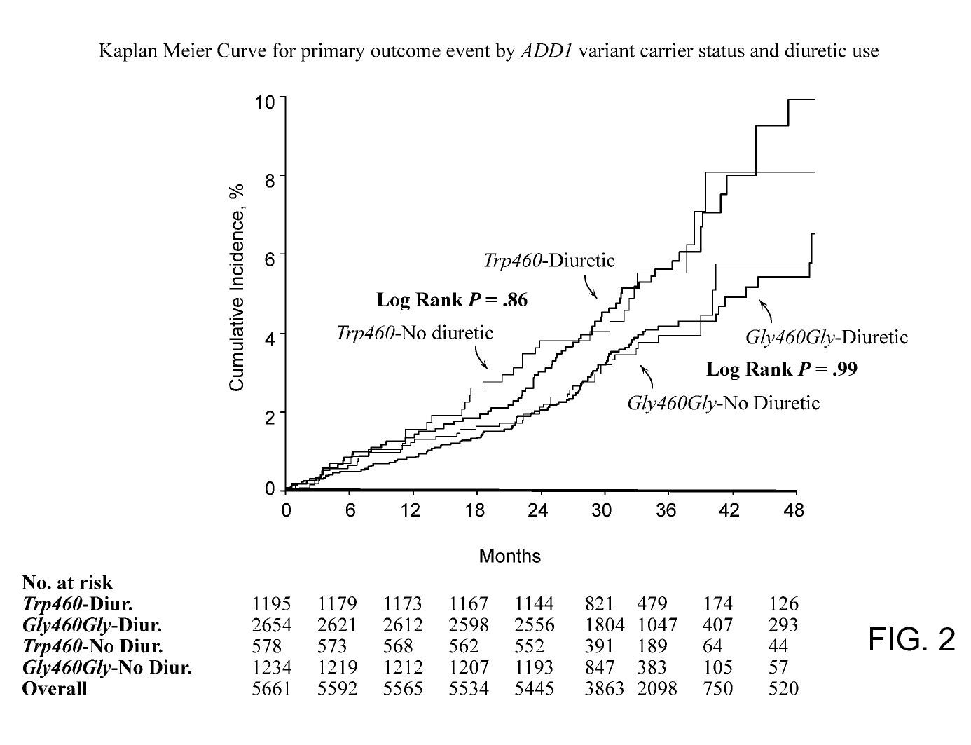

Time to primary outcome event was not different between users and non-users of diuretics regardless of ADD1 variant carrier status (Log Rank P, 0.86 and 0.99 for variant carriers and wild time homozygotes, respectively) (FIG. 2). Accordingly, the interaction between ADD1 carrier status and diuretics was not significant in the adjusted Cox PH model (HR 1.02, 95% CI 0.62-1.70, P=0.93). When calculated for nonfatal stroke or nonfatal myocardial infarction (the outcome used in a previous study showing an effect modification of diuretics by ADD1²⁶), the interaction term remained not significant (p=0.48). Additionally, the interactions of ADD1 carrier status with sex, ethnicity (Black vs. non-Black), age (>70y vs. ≦70y, as well as continuous), systolic blood presser (>160 mm Hg vs. ≦160 mm Hg, as well as continuous), and history of smoking were tested individually in the Cox proportional hazards model. The interaction term of ADD1 carrier status with ethnicity suggested a trend towards greater detrimental effect of ADD1 variant carrier status among Blacks as compared to other ethnicities (HR for interaction 1.98; P=0.09). None of the remaining interaction terms reached statistical significance (all P-values for interactions >0.3).

### Secondary Analyses

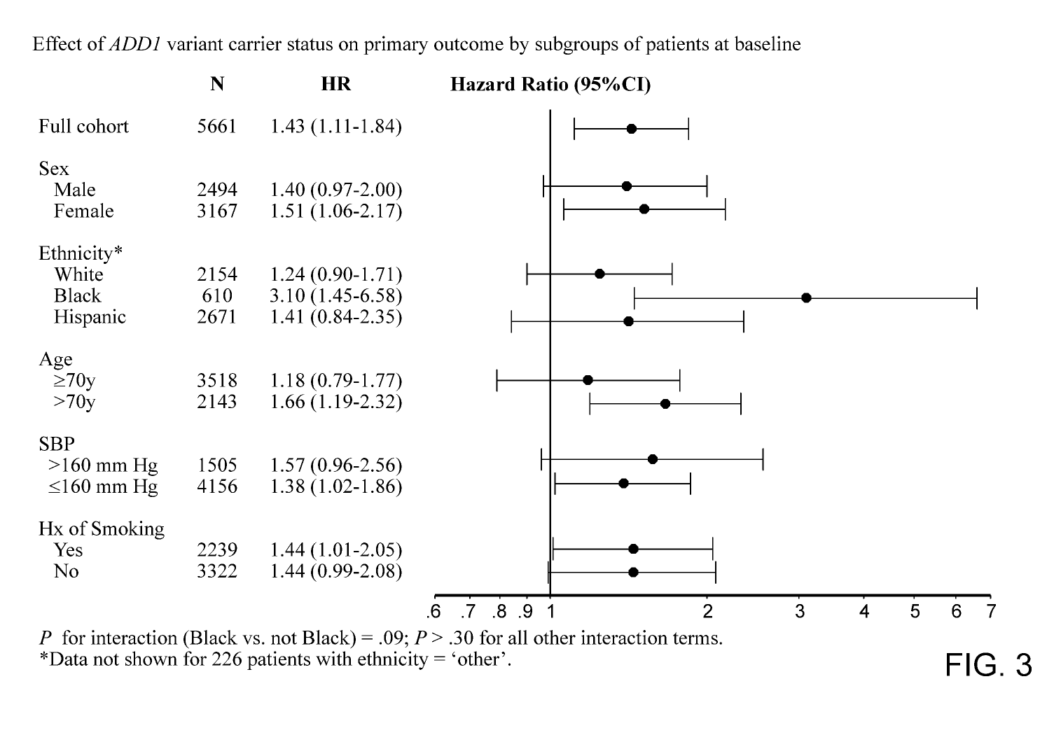

The hazard ratio for ADD1 variant carrier status remained consistent when the model additionally controlled for the percentage of trial visits with controlled blood pressure (HR 1.43, 95% CI 1.11-1.85, P=0.06). Adjusted hazard ratios for ADD1 variant carrier status stratified by sex, ethnicity, age (>70y vs. ≦70y), systolic blood pressure (>160 vs. ≦160), and smoking status are presented in FIG. 3.

Adjusted for significant covariates, an ANCOVA statistical analysis showed no difference between the number of strategy drugs, overall drugs, or diastolic blood pressure at the end of follow up between carriers of the ADD1 variant and wild type homozygotes (adjusted mean 1.88 vs. 1.92, P=0.19 for strategy antihypertensive drugs; adjusted mean 2.61 vs. 2.71, P=0.49 for total antihypertensive drugs; 76.9 vs. 76.9, P=0.93 for diastolic blood pressure). Carriers of the ADD1 variant had a slightly lower systolic blood pressure at the end of the study than wild type homozygotes (131.8 vs. 132.7, P=0.04).

The 3:1 ethnicity, age, and gender matched case-control dataset included 1,071 subjects compared to 5,979 subjects in the full INVEST-GENES cohort. Calculation of the ADD1 main-effect resulted in an adjusted hazard ratio/95% confidence intervals of 1.44 (1.12-1.86) for the case-control dataset, compared to hazard ratio/95% confidence intervals of 1.43 (1.11-1.84) for the full study cohort, suggesting that in future genetic association studies of the primary outcome within the INVEST-GENES, this case-control dataset may reduce genotyping requirements by a factor of >5 without apparent introduction of bias or meaningful loss in precision.

Results in Example 1 were obtained using the following methods and materials.

### Prospective Cohort

Prospective cohort study among 5,979 patients who participated in the INternational VErapamil SR-trandolapril STudy (INVEST) and provided genomic DNA. The INVEST randomized patients to either atenolol or verapamil SR, with diuretic (HCTZ) and/or trandolapril added if needed for blood pressure control or per treatment protocol. The primary outcome was defined as time to first occurrence of nonfatal stroke, nonfatal myocardial infarction, or all-cause death. Adjusted and unadjusted hazard ratios (HRs) and 95% confidence intervals (CIs) were calculated using Cox proportional hazards regression. 1,773 patients (31.3%) carried at least one copy of the ADD1 variant, 3,849 patients (68.0%) were exposed to diuretics, and 258 patients (4.6%) experienced a primary outcome event. ADD1 variant carriers were at higher risk for an event than wild type homozygotes (adjusted HR1.43; 95% CI 1.11-1.84; P=0.006). There was no interaction between the ADD1 gene and diuretic use (adjusted HR 1.02; 95% CI 0.2-1.70; P=0.93).

In hypertensive patients with cardiovascular disease, ADD1 variant carriers showed a 43% excess risk for a primary outcome event. The beneficial effect of diuretic use on risk of cardiovascular outcomes did not vary by ADD1 carrier status.

### Study Population and Interventions

The present study was pre-specified as part of INVEST-GENES, the GENEtic Substudy of the INternational VErapamil SR-trandolapril STudy (INVEST). After an extensive cardiovascular history and physical exam the INVEST randomly assigned 22,576 coronary artery disease patients ≧50 years old to either a verapamil SR- or an atenolol-based multidrug antihypertensive strategy. Trandolapril and hydrochlorothiazide (HCTZ) were specified as added agents, if needed for blood pressure control, with trandolapril added first in the verapamil SR strategy and HCTZ added first in the atenolol strategy. In both strategies, trandolapril was recommended for patients with heart failure, diabetes, or renal impairment. Between 1997 and 2003, 61,835 patient-years follow-up were accumulated and each strategy provided excellent blood pressure control (>70% of patients achieved blood pressure <140/90 mm Hg) without differences in blood pressure between the strategies. The strategies were equivalent in preventing the primary outcome defined as all-cause death, nonfatal myocardial infarction, or nonfatal stroke. All components of the primary outcome (death, nonfatal myocardial infarction, nonfatal stroke) were fully adjudicated by an independent adjudication committee (Pepine Jama. 2003; 290:2805-16). Further details on the design and results have been published (Pepine Jama. 2003; 290:2805-16; Pepine et al., J Am Coll Cardiol. 1998; 32:1228-37). Genetic samples were collected on 5,979 subjects from 184 sites in mainland US and Puerto Rico.

### Drug Exposure for Add-on Drugs

Drug exposure to primary strategy drugs (atenolol and verapamil SR) was defined by INVEST randomized treatment strategy. In contrast to the primary strategy drugs, patients were only exposed to added agents after failure to meet blood pressure goals or for trandolapril, if the patient had heart failure, renal insufficiency or diabetes. Therefore, exposure to HCTZ or trandolapril as part of the INVEST treatment protocol was operationalized as a time-varying covariate and coded 0 before exposure and 1 from the date of the first prescription to the end of the study follow up or event.

### Genotyping

Genomic DNA was isolated from buccal genetic samples by using commercially available kits (PureGene, Gentra Systems Inc., Minneapolis, Minn.) and normalized to 20 ng/ul. Genotyping for the ADD1 460Trp polymorphism was performed by polymerase chain reaction (PCR) followed by Pyrosequencing® (PSQ) (Langaee et al., Mutat Res. 2005; 573:96-102). The following primers were used for PCR reaction and Pyrosequencing: 5′-biotin-AAATACAGCGATGTGGAGGTTCC-3′ (PCR-Forward), 5′-CAGTTGGTAATACAG CTTGGCAC-3′ (PCR-Reverse) and sequencing primer 5′-TGCTTCCATTCTGCC-3′. The PCR mixture (12.50 μL) consisted of 6.25 μL HotStarTaq® Master Mix Kit (Qiagen Inc, Valencia, Calif.), 1 μL PCR primers (10 pmol/μL), 1 HL of dimethyl sulfoxide, 1.25 μL of H2O, and 40 ng of DNA. PCR was performed under the following conditions: 95° C. for 15 minutes; 40 cycles consisting of denaturation at 95° C. for 30 seconds, annealing at 62° C. for 40 seconds and extension at 72° C. for 30 seconds; and final extension for 7 minutes. Pyrosequencing was performed under standard conditions for sequence determination and allele designation in a Biotage PSQ HS 96 System, and data were captured with PSQ HS 96 SNP software. The genotype and primary event data have been deposited in the Pharmacogenomics Knowledge Base (www.pharmgkb.org; accession Nos. PS205467 and PS205547).

### Statistical Analysis

Initially, the analysis was conducted using three ADD1 genotypes, Gly460Gly, Gly460Trp, and Trp460Trp. There were no statistically significant differences between the effects of Gly460Trp and Trp460Trp on primary outcome (event rates were 5.7% (9 of 152) and 5.9% (92 of 1,621) for Gly460Trp and Trp460Trp, respectively; X²=0.016; p=0.90). Therefore, due to the rarity of the Trp460Trp genotype (2.7%, 152 of 5,661), study subjects were classified as either ADD1 wild-type homozygotes (Gly460Gly) or as ADD1 variant carriers (Gly460Trp or Trp460Trp). Baseline characteristics between ADD1 variant carriers and wild-type homozygotes were compared using t-test for continuous and Chi-squared test for categorical variables, respectively. Hardy-Weinberg equilibrium (HWE) within each ethnic group was tested with Chi-squared tests. Kaplan Meier curves were estimated to compare time to first primary outcome event by ADD1 variant carrier status as well as ADD1 variant carrier status and diuretic use. For hypothesis 1, the effect of ADD1 variant carrier status on incidence of primary outcome events was investigated using a Cox proportional hazards (PH) model. Adjusted and unadjusted hazard ratios (HRs) and 95% confidence intervals (CI) were calculated. The model controlled for the following covariates: Age, ethnicity, sex, INVEST treatment strategy, use of HCTZ and/or trandolapril, as well as previous myocardial infarction, stroke or transient ischemic attack (TIA), smoking, diabetes, renal insufficiency, heart failure, body mass index, and systolic blood pressure. Time-varying exposure was used for HCTZ and trandolapril since these drugs were added at different times for each individual patient. Additionally, the Cox PH model was used to calculate adjusted HRs and CIs for ADD1 variant carrier status by sex, ethnicity, age (>70y vs. ≦70y), systolic blood pressure (>160 mmHg vs. ≦160 mmHg), treatment strategy, smoking status, and for components of the primary outcome (all cause death, CARDIOVASCULAR death, non-fatal myocardial infarction, non-fatal stroke, total myocardial infarction, and total stroke).

The gene-diuretic interaction (hypothesis 2) was tested as the interaction term between ADD1 variant carrier status with time-varying exposure to diuretics in the Cox PH model. The interactions of the ADD1 variant with sex, ethnicity, age, systolic blood pressure, and smoking status were tested using an analogous approach.

In addition, analysis of covariance (ANCOVA) was used to test for differences in the number of strategy antihypertensive drugs, total antihypertensive drugs, as well as blood pressure at the end of follow up between carriers of the ADD1 variant and wild type homozygotes controlling for significant covariates.

In order to determine whether potential genotyping efficiencies could be realized for ongoing and future studies within the INVEST-GENES, we created a case-control dataset containing all patients with a primary outcome event (cases) and a 3:1 group of event free patients (controls), who were frequency matched to the cases on ethnicity, gender and age. Unadjusted and adjusted HRs for ADD1 carrier status were computed for the case-control dataset and compared to those of the full study cohort. SAS version 9.1.3 (SAS institute, Cary, N.C.) was used for data management and statistical analysis.

### Example 2

### KCNMB1 Genotype Influences Response to Verapamil SR and Adverse Outcomes

Baseline characteristics for all INVEST-GENES patients are shown in Table 5.

TABLE 5
Baseline characteristics among those with complete Val110Leu
genotype*

| | INVEST- | | | | | |
|---|---|---|---|---|---|---|
| | GENES | | Cases | | Controls | |
| Characteristic | (N = 5486) | | (N = 255) | | (N = 798) | |
| Age, mean (SD), years | 66.1 | (9.7) | 71.6 | (9.9) | 69.9 | (9.3) |
| Women | 3047 | (55.5) | 131 | (51.4) | 400 | (50.1) |
| BP, mean (SD), | | | | | | |
| mmHg | | | | | | |
| Systolic | 148.0 | (18.4) | 150.7 | (19.0) | 147.4 | (19.1) |
| Diastolic | 85.4 | (10.7) | 83.6 | (11.1) | 83.5 | (11.2) |
| Race/ethnicity | | | | | | |
| White | 2076 | (37.8) | 152 | (59.6) | 468 | (58.7) |
| Black | 588 | (10.7) | 32 | (12.6) | 93 | (11.7) |
| Hispanic | 2598 | (47.4) | 63 | (24.7) | 219 | (27.4) |
| Other/multiracial | 224 | (4.1) | 8 | (3.1) | 18 | (2.2) |
| BMI, mean (SD), | 29.3 | (5.5) | 27.5 | (4.8) | 29.1 | (5.6) |
| kg/m² | | | | | | |

| | | | |
|---|---|---|---|
| Lys65 allele frequency | n/a | 0.135 | 0.11 |
| Leu110 allele | 0.09 | 0.06 | 0.09 |
| frequency | | | |

Past Medical History

| | | | | | | |
|---|---|---|---|---|---|---|
| Myocardial infarction | 1281 | (23.4) | 95 | (37.3) | 236 | (29.6) |
| Angina pectoris | 4070 | (74.2) | 151 | (59.2) | 503 | (63.0) |
| Revascularization >1 | 783 | (14.3) | 62 | (24.3) | 149 | (18.7) |
| month ago | | | | | | |
| Stroke/TIA | 381 | (6.9) | 36 | (14.1) | 73 | (9.2) |
| Left ventricular | 823 | (15.0) | 45 | (17.7) | 139 | (17.4) |
| hypertrophy | | | | | | |
| Heart failure | 179 | (3.3) | 28 | (10.3) | 32 | (4.0) |
| (class I-III) | | | | | | |
| Peripheral vascular | 607 | (11.1) | 43 | (16.9) | 95 | (11.9) |
| disease | | | | | | |
| Smoking | | | | | | |
| Past | 2265 | (41.3) | 130 | (51.0) | 364 | (45.6) |
| Within 30 days | 558 | (10.2) | 32 | (12.6) | 81 | (10.2) |
| Never | 3221 | (58.7) | 125 | (49.0) | 434 | (54.4) |
| Diabetes‡ | 1542 | (28.1) | 107 | (39.5) | 232 | (29.1) |
| Hypercholesterolemia‡ | 2998 | (54.7) | 159 | (62.4) | 497 | (62.3) |
| Renal impairment† | 85 | (1.6) | 14 | (5.2) | 19 | (2.4) |
| Cancer | 222 | (4.1) | 20 | (7.8) | 46 | (5.8) |

Medication

| | | | | | | |
|---|---|---|---|---|---|---|
| Aspirin/other | 2493 | (45.4) | 159 | (62.4) | 457 | (57.3) |
| antiplatelet agent | | | | | | |
| Antidiabetic | 1351 | (24.6) | 85 | (33.3) | 195 | (24.4) |
| medication | | | | | | |
| Any lipid-lowering | 1985 | (36.2) | 104 | (40.8) | 338 | (42.4) |
| agent | | | | | | |
| Nitrates | 1540 | (28.1) | 91 | (35.7) | 243 | (30.5) |

Abbreviations:
n/a: not applicable;
BMI: body mass index;
Revascularization: CABG: coronary artery bypass graft or PTCA;
*Values expressed as number (percentage) unless otherwise indicated. Percentages may not equal 100 due to rounding.
‡History of or currently taking antidiabetic or lipid-lowering medications.
†History of or currently have elevated serum creatinine level but less than 4 mg/dL (<354 μmol/L).

As expected due to the entry criterion of ≧50 years of age, the patients were elderly with a mean age of 66 years. Over half of the patients were female, with a high percentage of Hispanics. Baseline characteristics were similar when compared by codon 110 genotypes, with the exception of the variant allele being slightly more frequent in Hispanic and Black patients than White patients. In addition, hypercholesterolemia was present in 55% of Val110Val patients compared to 51.3% of Leu110 carriers (p=0.03). Baseline characteristics for 1,071 patients selected as cases and controls are similar to the overall INVEST-GENES cohort (Table 5).

Genotyping was successful for codon 65 in 99% of the blood pressure response cohort and 97% of cases (98% overall). For codon 110, genotyping was conducted in the entire INVEST-GENES cohort and was successful in 99% of the blood pressure response cohort and 99% of cases (92% overall). For quality control purposes, 470 samples were genotyped in duplicate with a 99% concordance rate.

All but three of those patients missing genotypes for codon 110 were patients in the overall cohort who did not experience an event and additional efforts to successfully genotype these samples was deemed unnecessary given the limited power gained with the addition of 490 controls to the existing controls with genotype data. Codon 65 genotype frequencies did not deviate from Hardy-Weinberg equilibrium for any of the racial/ethnic groups for codon 65 (p>0.14 for all groups). Codon 110 genotypes did not deviate from Hardy-Weinberg equilibrium in Hispanic or Black patients. Codon 110 deviated among White individuals (p<0.01). Genotype frequencies are shown in Table 6.

TABLE 6
KCNMB1 genotype frequency by race/ethnicity and in INVEST-GENES

| | INVEST-GENES | White | Hispanic | Black | Other |
|---|---|---|---|---|---|
| Glu65Glu | 1412 (77%) | 743 (81%) | 466 (73%) | 153 (76%) | 50 (79%) |
| Glu65Lys | 392 (22%) | 165 (18%) | 169 (26%) | 45 (23%) | 13 (21%) |
| Lys65Lys | 20 (1%) | 9 (1%) | 9 (1%) | 2 (1%) | 0 |
| Lys65 allele | 0.12 | 0.10 | 0.14 | 0.13 | 0.10 |
| frequency | | | | | |
| Val110Val | 4591 (84%) | 1782 (86%) | 2143 (83%) | 480 (82%) | 186 (83%) |
| Val110Leu | 843 (15%) | 273 (13%) | 432 (17%) | 103 (18%) | 35 (16%) |
| Leu110Leu | 52 (1%) | 21 (1%) | 23 (1%) | 5 (1%) | 3 (1%) |
| Leu110 | 0.09 | 0.08 | 0.09 | 0.10 | 0.09 |
| allele | | | | | |
| frequency | | | | | |

The two SNPs were in strong linkage disequilibrium in White patients (D′=1), but weakly linked in Hispanic and Black patients (D′=0.52 and D′=0.55, respectively). Baseline demographic characteristics did not differ between the 5,486 individuals from INVEST-GENES in whom codon 110 genotyping was successful and the 493 individuals in whom genotyping was not successful.

Because sample sizes for the various analyses were determined by patients' participation in INVEST, statistical power was estimated post-hoc for each subgroup of interest. For the verapamil SR monotherapy patients, there was 80% power with a two-sided a of 0.05 to detect an 8 mmHg difference in treatment systolic blood pressure between variant carriers and homozygous wild-type individuals for codon 65 and a 9 mmHg difference for codon 110. For the entire blood pressure response cohort of patients, there was 80% power to detect a 5 mmHg difference in systolic blood pressure for both codon 65 and codon 110. For the case-control logistic regression, there was 80% power to detect an alternative hypothesis with an odds ratio of 1.58 for codon 65 and 1.6 for codon 110. For the entire INVEST-GENES cohort, there was 80% power to detect a hazard ratio of 1.55 for codon 110.

### BP Response to Verapamil SR

Among INVEST-GENES patients, 163 met criteria for verapamil SR monotherapy patients and 603 met criteria for being considered in the overall blood pressure response cohort. In general, the blood pressure response cohort was similar to the entire INVEST-GENES cohort with a few exceptions. White patients made up 47% of verapamil SR monotherapy patients compared to 39% of the overall blood pressure response cohort and 41% of overall INVEST-GENES. In addition, mean baseline blood pressure was slightly higher in verapamil SR monotherapy patients at 157/91±16/8 mmHg compared to 149/86±18/10 in the overall blood pressure response group and 148/85±18/11 in the entire INVEST-GENES cohort (148/85±18/11).

### Verapamil SR-Mediated Change in Blood Pressure

Among verapamil SR monotherapy patients, mean systolic blood pressure response to verapamil SR treatment at six weeks adjusted for baseline systolic blood pressure, age, sex, race, and body mass index was 139±1.3 mmHg in Glu65Glu patients, compared to β2±4.0 mmHg in Lys65 carriers, p=0.077. When compared by codon 110 genotype, treatment systolic blood pressure was 139±1.2 mmHg in Val110Val patients and 133±4.0 mmHg in Leu110 carriers, p=0.147.

When patients with stable background therapy were considered in addition to the verapamil monotherapy patients, systolic blood pressure response did not differ by codon 65 or codon 110 genotype. Systolic blood pressure was 136±0.9 mmHg in Glu65Glu patients, 135±1.8 mmHg in Lys65 carriers, p=0.595 and 135±0.7 mmHg in Val110Val patients, 135±1.9 mmHg in Leu110 carriers, p=0.764. A significant interaction between genotype and age or sex was not found.

When codon 65 and codon 110 were considered jointly as haplotypes, the analysis was less informative than either single SNP analysis.

### Time to Blood Pressure Control

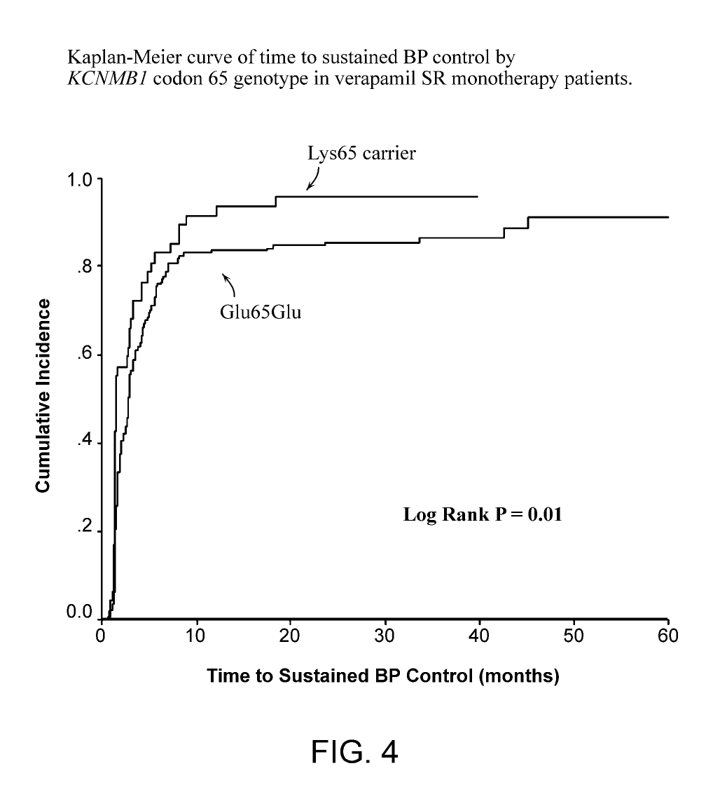

For the time to blood pressure control analyses, 218 patients were available in the verapamil SR monotherapy and 791 were available in the entire blood pressure response group. The median time to achieve blood pressure control (first visit with systolic blood pressure <140 and diastolic blood pressure <90 mmHg and 50% of visits thereafter) among verapamil SR monotherapy patients was 2.83 (IQR 4.17) months in Glu65Glu patients and only 1.47 (IQR 2.77) months in Lys65 carriers, p=0.01 (FIG. 4). Median time to blood pressure control in the entire blood pressure response cohort was 2.8 (IQR 1.33) months in Glu65Glu patients and 2.0 (IQR 1.40) months in Lys65 carriers, p=0.06. Time to blood pressure control did not differ by codon 110 genotype in verapamil SR monotherapy patients or among all blood pressure response patients (data not shown).

### Number of Drugs at Time of Blood Pressure Control

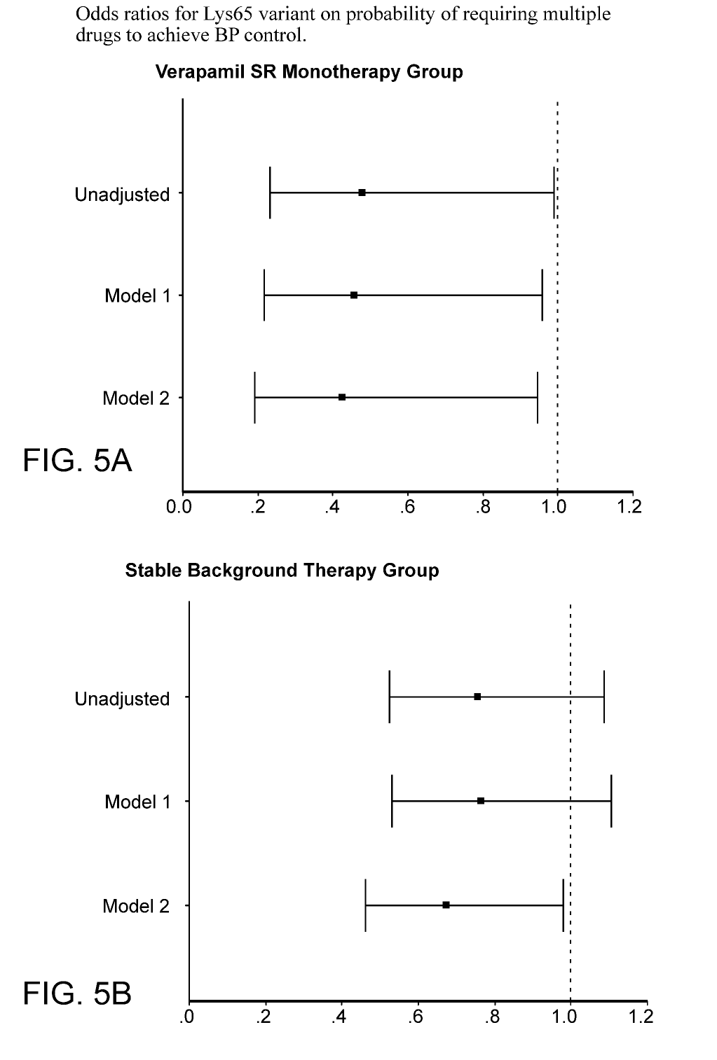

Consistent with the observation that less time was required to achieve blood pressure control, Lys65 variant carrier status was significantly associated with the need for fewer drugs to achieve blood pressure control (OR 0.48 95% CI 0.23, 0.99) in the verapamil SR monotherapy group (FIG. 5A). Additionally, as multiple covariates were added to the model including demographics and disease states, the effect of Lys65 variant carrier status remained significant. The effect of Lys65 had similar trends in the entire blood pressure response cohort, but was less pronounced than in the verapamil SR monotherapy group (FIG. 5B).

### KCNMB1Genotype Effected Primary Outcome

Case-Control Group

In the case-control population, the Leu110 variant was associated with a 33% reduced risk of primary outcome (OR 0.66, 95% CI 0.43-1.01). Adjustment for all pre-specified covariates did not affect this association (OR 0.64, 95% CI 0.41-1.01). When the ancestral information markers were included in the model, a similar association was found (OR 0.59, 95% CI 0.34-0.99), suggesting that these findings are not spurious due to population stratification. No significant association between codon 65 and occurrence of primary outcomes was observed (OR 1.15, 95% CI 0.83-1.61).

### Entire INVEST-GENES Cohort

In order to ensure that the case-control study design was representative of the entire INVEST-GENES cohort and that important associations were not missed because of lack of power, all the INVEST-GENES patients were genotyped for the Val110Leu polymorphism.

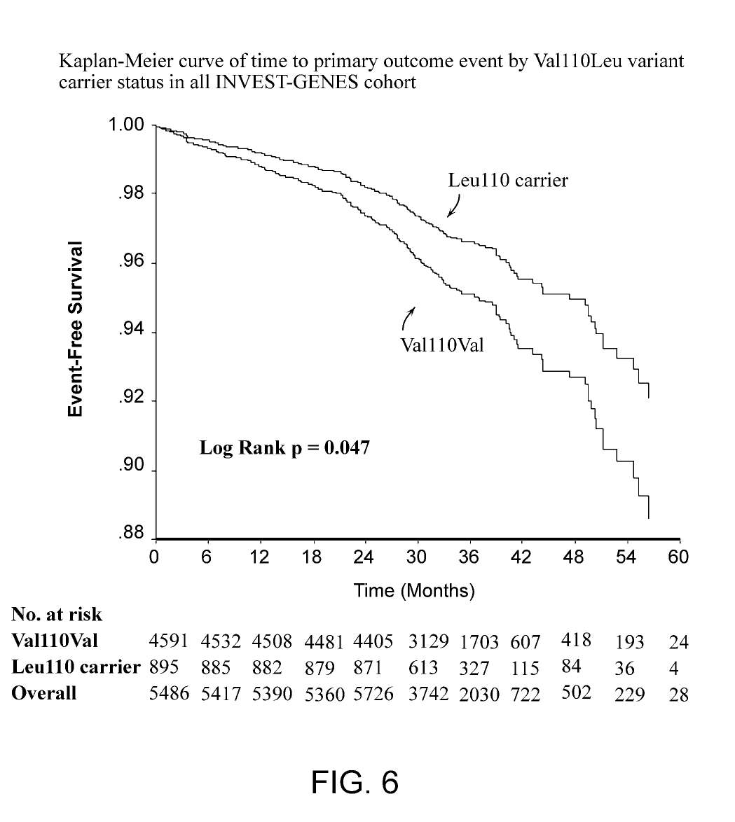

Unadjusted for differences in baseline characteristics, codon 110 variant carrier state was associated with a 32% lower risk of primary outcome than non carriers (HR 0.68 95% CI 0.47-0.99) (FIG. 6). After full adjustment for pre-specified covariates, this risk still trended toward significance (HR 0.72 95% CI 0.49-1.05) Results were consistent when the ancestral information markers were included in the modeling instead of the race/ethnicity term (HR 0.66 95% CI 0.42, 1.03). Haplotype analysis did not provide additional information over consideration of genotype alone. As a secondary analysis, the components of the primary outcome were tested individually and a significant reduction in the risk of nonfatal myocardial infarction in Leu110 variant carriers was found in the unadjusted model that trended toward significance in the fully adjusted model (Table 7).

TABLE 7
Primary and secondary outcomes by codon 110 genotype

| | Leu110 | | Unadjusted HR | Adjusted* HR |
|---|---|---|---|---|
| | carrier | Val110Val | (95% CI) for variant | (95% CI) for variant |
| | (n = 895) | (n = 4591) | carrier status | carrier status |
| Primary outcome | 30 (0.55) | 225 (4.1) | 0.682 (0.466, 0.998) | 0.719 (0.491, 1.053) |
| event | | | | |
| Secondary | | | | |
| outcomes | | | | |
| Death (all cause) | 13 (0.24) | 85 (1.6) | 0.792 (0.442, 1.419) | 0.814 (0.453, 1.463) |
| Nonfatal MI | 6 (0.11) | 74 (1.4) | 0.415 (0.181, 0.954) | 0.448 (0.195, 1.031) |
| Nonfatal stroke | 13 (0.24) | 72 (1.3) | 0.925 (0.513, 1.670) | 0.984 (0.544, 1.779) |

*Adjusted for: age, sex, race/ethnicity, body mass index, smoking, INVEST treatment strategy, previous myocardial infarction, previous stroke, heart failure, diabetes, renal insufficiency, baseline systolic blood pressure, diuretic use, and ACE inhibitor use.

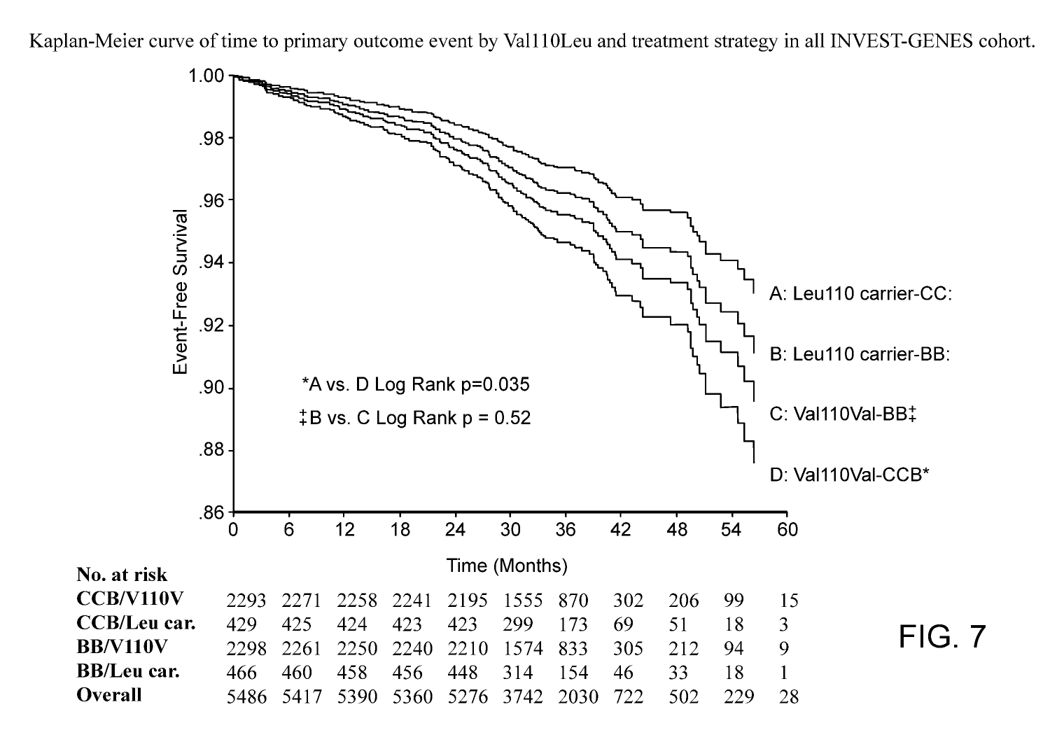

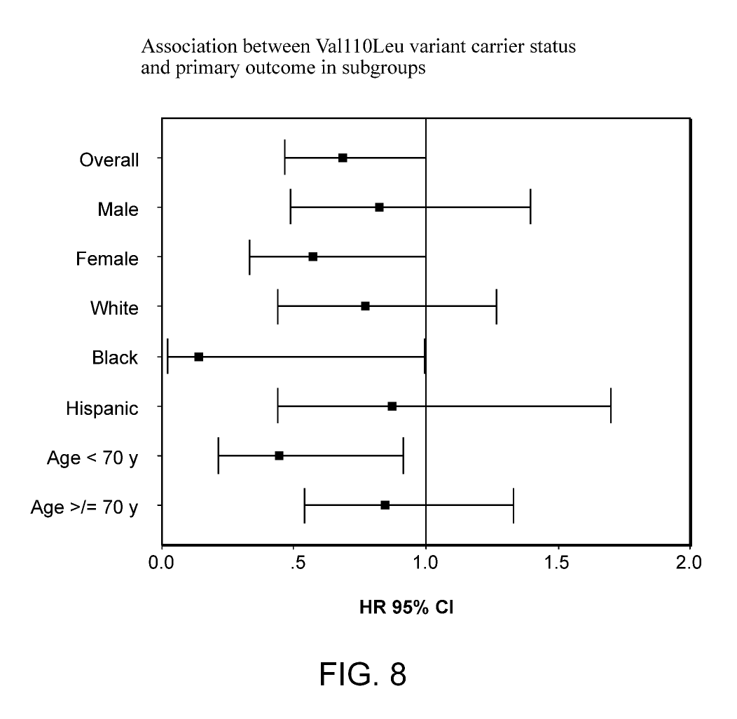

Although the interaction term was not significant between treatment strategy and codon 110 genotype, when the analyses were conducted separately by treatment strategy, the protective effect of the KCNMB1 variant allele appeared to be confined to those randomized to verapamil SR (HR 0.55 95% CI 0.31-0.97), with no benefit in the atenolol-treated group (HR 0.84 95% CI 0.51-1.41) (FIG. 7). The association between codon 110 genotype and adverse outcomes was more prominent in women (HR 0.57 95% CI 0.33-0.99), individuals <70 years of age (HR 0.45 95% CI 0.22-0.92), and Black individuals HR 0.14 95% CI 0.02-1.00) (FIG. 8).

### Val110Leu Functional Assessment

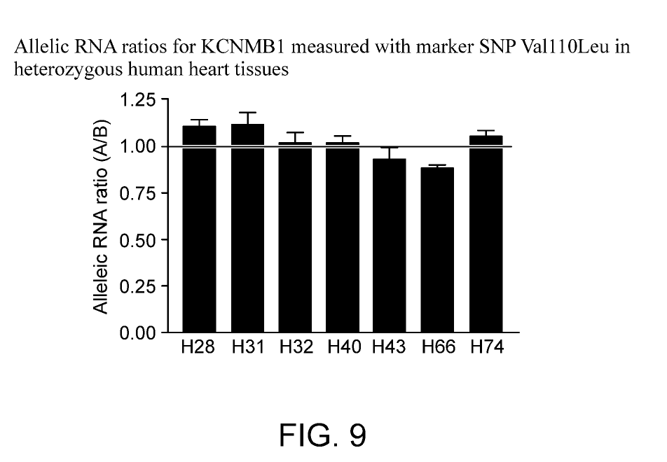

Of 65 heart tissue samples, 7 samples were heterozygous for Val110Leu. Allelic expression in these heterozygous DNA and RNA (converted to cDNA) samples was measured. The presence of any polymorphism that affects transcription or mRNA processing should result in discrepant DNA and cDNA allelic ratios (AEI). Shown in FIG. 9, RNA ratios from all 7 samples did not differ from DNA ratios when using Val110Leu as a marker, indicating that this non-synonymous SNP was not associated with different mRNA levels of KCNMB1 in cardiac tissues. Using another two SNPs in 3′-UTR (rs2656842, rs2656841) as marker SNPs, we confirmed this result in three samples also heterozygous for these two SNPs.

These findings indicate that in hypertensive patients with coronary artery disease, two non-synonymous polymorphisms in the KCNMB1 gene, Glu65Lys and Val110Leu, likely account for a component of the interpatient variability in blood pressure response to verapamil SR. Additionally, these findings indicated that the Val110Leu polymorphism is associated with adverse outcomes. These conclusions are based on results of several different types of analyses. First, Lys65 variant carriers achieved blood pressure goals more rapidly than Glu65 homozygote individuals in the verapamil SR monotherapy group and required fewer drugs in order to achieve blood pressure control. These results were consistent with functional studies of this polymorphism indicating that the variant BK channel has enhanced calcium sensitivity (Fernandez-Fernandez et al., J Clin Invest 2004; 113(7):1032-9) and would be predicted to have enhanced responsiveness to calcium channel blockers. All of the blood pressure response analyses (i.e. change in systolic blood pressure at six weeks, time to blood pressure control, and number of drugs needed for blood pressure control) consistently identified Lys65 variant carriers as having the most favorable blood pressure response to verapamil SR in patients on verapamil SR monotherapy.

The second major finding reported herein was that the Leu110 variant allele is associated with a decreased risk of adverse outcome among patients with hypertension and coronary artery disease. Interestingly, this finding was significant only in the patients randomized to a verapamil SR-based treatment strategy, suggesting a pharmacogenetic effect, with the majority of the protective effect of the variant occurring only when the patients are treated with a calcium channel blocker. These findings suggest that hypertensive coronary artery disease patients who are Leu110 carriers should receive a calcium channel blocker.

The subgroup analyses, suggested that greater protective effective effect associated with the Leu110 variant allele was found in women, individuals less than 70 years, and Black individuals.

These results indicated that variability in KCNMB1 is associated with the antihypertensive response to verapamil SR, and also adverse cardiovascular outcomes in hypertensive patients with coronary artery disease. Of particular interest regarding the latter finding is that the protective effect of the variant allele was largely confined to who received verapamil SR monotherapy. This is the first study evaluating the influence of KCNMB1 on the antihypertensive response to verapamil SR and highlights the importance of consideration of pharmacodynamic pathway genes.

Results obtained in Example 2 were carried out using the following materials and methods.

### INVEST Clinical Trial Design Overview

The INVEST evaluated blood pressure and adverse outcomes occurring with either an atenolol-based or a verapamil SR-based hypertension treatment strategy in 22,576 patients with documented coronary artery disease and hypertension (Pepine et al., J Am Coll Cardiol 1998; 32(5):1228-37). The design, protocol, and primary outcome have been published in detail elsewhere (Pepine et al., JAMA 2003; 290(21):2805-16; Pepine et al., J Am Coll Cardiol 1998; 32(5):1228-37). Briefly, the protocol required patients to be seen at baseline, 6, 12, 18, and 24 weeks, and then every 6 months thereafter until two years after the last patient was enrolled. At each visit, patients had blood pressure and heart rate measured, clinical assessment, and additional antihypertensive medications added as needed to meet JNC VI blood pressure goals (Pepine et al., JAMA 2003; 290(21):2805-16; Arch Intern Med 1997; 157(21):2413-46). The primary outcome was the first occurrence of death (all cause), nonfatal myocardial infarction, or nonfatal stroke. Clinical Trial Registration Identifier: NCT00133692, URL: http://clinicaltrials.gov/ct/gui/show/NCT00133692?order=5.

### INVEST-GENES

Genetic samples were collected from 5,979 INVEST patients residing in mainland United States and Puerto Rico. Genomic DNA was collected using buccal cells from mouthwash samples as previously described (Andrisin et al., Pharmacotherapy 2002; 22(8):954-60).

As in most recent hypertension trials conducted in high risk patients, the INVEST protocol permitted entry of patients receiving background antihypertensive therapy, as well as those receiving no drug treatment. For the blood pressure response to verapamil SR analysis, patients in whom changes in blood pressures were attributed to verapamil SR were genotyped. These were patients entering INVEST with untreated hypertension who were prescribed verapamil SR monotherapy at the first study visit (verapamil SR monotherapy group) and patients receiving antihypertensive therapy at entry which was maintained, with addition of verapamil SR as the only change to their antihypertensive regimen at the first study visit (stable background therapy group). Blood pressure response analyses were conducted in the entire blood pressure response cohort (verapamil SR monotherapy plus stable background therapy groups) and in the verapamil SR monotherapy patients separately.

### Tissue Samples

Given positive association studies with codon 110 and a lack of published functional data, allelic expression imbalance (AEI) studies were undertaken as an initial functional assessment. Allelic expression imbalance is observed in target tissues in subjects where the studied gene harbors a functional polymorphism that affects gene regulation and mRNA processing. Polymorphisms in coding regions (synonymous or nonsynomous) in a majority of cases affects mRNA folding, and hence has an inherent potential to affect mRNA processing and functions (Johnson et al., Pharmacol Ther 2005; 106(1):19-38). Alternatively, such polymorphisms could be in linkage disequilibrium with regulatory polymorphisms. Given the limited tissues of expression for this protein, and the difficulties in studying ion channel function in human target tissues, this approach was chosen as a starting point of the potential functional effects of this polymorphism.

Sixty-five left ventricle heart tissues were obtained from patients undergoing heart transplantation at Ohio State University Medical Center. Genomic DNA and total RNA were prepared from these tissues as described previously (Johnson et al., Pharmacol Ther 2005; 106(1):19-38; Pinsonneault et al., J Pharmacol Exp Ther 2004; 311(3):1088-96). Complementary DNA (cDNA) was generated from 0.5 μg total RNA using oligo-dT and gene specific primers (5′-GATTGGACTGGAAGAGTGGG) as described in (Johnson et al., Pharmacol Ther 2005; 106(1):19-38; Pinsonneault et al., J Pharmacol Exp Ther 2004; 311(3):1088-96).

### Nested Epidemiological Studies

Three studies within the INVEST-GENES patient population. First, a cohort study was conducted where the outcome of interest was systolic blood pressure response to verapamil SR at six weeks and the secondary outcomes were time to blood pressure control and number of drugs required at time of blood pressure control, compared by KCNMB1 genotype.

Second, a nested case-control study was conducted among the 258 INVEST-GENES patients who experienced a primary outcome event (death, nonfatal myocardial infarction, or nonfatal stroke) during study follow-up (cases). A total of 813 individuals who did not have an event during study follow-up were frequency matched in a ratio of approximately 3:1 to cases for age, sex, and race/ethnicity (controls).

Last, because codon 110 trended toward significance in the case control study, a cohort study of all INVEST-GENES patients was conducted to evaluate the association of codon 110 genotype with the occurrence of the primary outcome. This cohort study was conducted in order to evaluate the appropriateness of our case control sample and to estimate the probability of missing a significant relationship with codon 110, if indeed one existed, because of a lack of power.

### Genotyping

Genomic DNA was extracted from buccal cells collected in mouthwash samples according to standard protocols (Andrisin et al., Pharmacotherapy 2002; 22(8):954-60). The Glu65Lys and Val110Leu polymorphisms were genotyped by pyrosequencing (PSQ HS 96A) or Taqman® methods. The PSQ HS 96 genotyping platform (Biotage AB, Uppsala, Sweden) was used for the pyrosequencing assay (primer sequences available upon request). The codon 65 and codon 110 PCR reactions were carried out using HotStar Taq mix, 10 pmmol each of forward and reverse primers, water, and 20 ng of genomic DNA. The annealing temperature was 58° for codon 65 and 63° for codon 110. The Applied Biosystems 7900 HT SNP genotyping platform was used for the Taqman® assay. The PCR primers and probes for KCNMB1 codon 65 and 110 assays (IDs C_{—}302618_{—}10, and C_{—}3026206_{—}1) were purchased from Applied Biosystems (Applied Biosystems, Foster City, USA). 5 μL reactions in 384-well plate were prepared and the assays were performed and analyzed according to the manufacture's recommendations.

AIMs were genotyped using either allele-specific PCR with universal energy transfer labeled primers (Myakishev et al., Genome Res 2001; 11((1): 163-9) or competitive allele specific PCR at Prevention Genetics (Marshfield, Wis.).

Haplotypes were computationally derived and pairwise linkage disequilibrium (D′) calculated separately for each racial/ethnic group using Polymorphism and Haplotype Analysis Suite (http://ilya.wustl.edu/˜pgrn/programs.html).

Quantitative Analysis of Allelic Ratios in Genomic DNA and mRNA Using SNaPshot

In order to determine whether Val110Leu affects mRNA level in heart tissue, this SNP was used as a marker for measuring the amount of mRNA derived from one allele over the other (allelic RNA ratio) in heterozygous samples. Deviation of RNA ratios from DNA ratios demonstrated the presence of allelic expression imbalance, as an indicator of cis-acting functional polymorphisms affecting gene regulation and mRNA processing and turnover (Johnson et al., Pharmacol Ther 2005; 106(1):19-38; Pinsonneault et al., J Pharmacol Exp Ther 2004; 311(3):1088-96; Zhang et al., J Biol Chem 2005; 280(38):32618-24). Details of the SNaPshot assay procedure have been published (Johnson et al., Pharmacol Ther 2005; 106(1):19-38; Pinsonneault et al., J Pharmacol Exp Ther 2004; 311(3):1088-96; Zhang et al., J Biol Chem 2005; 280(38):32618-24). Briefly, a segment of DNA or cDNA (˜100 bp) surrounding the marker SNP was amplified by PCR using forward primer (5′-TGCTCCTACATCCCAGGCA) and reverse primer (5′-AATTTGGCTCTGACCTTCTCC). Then the PCR product was subjected to a primer extension assay (SNaPshot, Applied Biosystems) using an extension primer (5′-GCCGTCTGGTAATTGTCCA) designed to anneal to the amplified DNA adjacent to the SNP site. Allelic DNA ratios, normalized to 1, served as internal control. Allelic mRNA ratios were normalized by DNA ratios.

### Statistical Analysis

Baseline characteristics were compared by genotype using chi-square or ANOVA, as appropriate. Because of the low minor allele frequency for both the codon 65 and codon 110 SNPs, we decided a priori to divide patients assuming a dominant model of inheritance with heterozygote patients pooled with the homozygous variant patients for all analyses. Hardy-Weinberg equilibrium was calculated separately by race/ethnicity using chi-square test with one degree of freedom. All statistical analyses were conducted using SAS version 9.1 (Cary, N.C.) or SPSS version 11.5 (Chicago, Ill.). A p<0.05 (two-sided) was considered significant for all analyses. Systolic blood pressure response after six weeks of verapamil SR therapy was compared by genotype using a general linear model with genotype included as a fixed effect adjusted for prespecified baseline covariates of age, sex, race/ethnicity, blood pressure, and body mass index, all of which in univariable analysis had a p<0.2 in either verapamil SR monotherapy or all blood pressure response patients. Treatment blood pressure was estimated using least square means (adjusted for above covariates)±standard error are presented. Secondary blood pressure analyses included time to blood pressure control, defined as the time after receiving drug when blood pressure control (<140/90 mmHg) was achieved and maintained for at least 50% of subsequent visits, and number of drugs required at time of blood pressure control. Kaplan Meier analysis was used to estimate time to blood pressure control and comparison between genotype groups were made using a log-rank test. Cox regression modeling using forward inclusion was also performed with the prespecified covariates. Number of drugs at time of blood pressure control was assessed using a cumulative logit model with factors known to influence the number of antihypertensive medications required including the prespecified covariates plus history of renal insufficiency, heart failure, and diabetes. Percent change in systolic blood pressure in response to verapamil SR was compared among haplotypes (0, 1, or 2 copies) using test for trend.

Unadjusted and adjusted odds ratios (ORs) and 95% confidence intervals (CIs) for occurrence of the primary outcome were calculated using logistic regression for the case-control group. The model contained the following baseline covariates used for primary INVEST analyses: age, sex, race/ethnicity, body mass index, smoking, INVEST treatment strategy, previous myocardial infarction, previous stroke, heart failure, diabetes, renal insufficiency, baseline systolic blood pressure, diuretic use, and ACE inhibitor use, plus codon 65 and 110 genotype, and the interaction term between genotype and strategy assignment.

A Cox proportional hazards model using forward inclusion with all of the above variables as pre-specified covariates was used to evaluate the effect of codon 110 genotype on outcomes and Kaplan Meier curves were estimated. Because the INVEST protocol called for diuretic (HCTZ) and ACE inhibitor (trandolapril) therapy to be added to the primary study drugs for patients failing to achieve blood pressure goals with primary agents or to those with heart failure, renal insufficiency or diabetes (for trandolapril), these variables were treated as time-dependent covariates in the Cox proportional hazards model. The model was also conducted separately by study strategy (i.e. in the verapamil SR-based group and in the atenolol-based group) and in subgroups by age (<70 years and ≧70 years), sex, and race/ethnicity.

To control for the potential of population stratification in our racially and ethnically diverse population, a total panel of 87 ancestry informative markers (AIMs) was used, selected to show large allele frequency differences across three parental populations (West Africans, Indigenous Americans, and Europeans) selected from a large panel of over 10,000 SNPs (Shriver et al., Hum Genomics 2005; 2(2):81-9_. Maximum likelihood was then used to estimate each patient's individual genomic ancestry proportions on these three axes and these terms were included in statistical models instead of the race/ethnicity term.

### Example 3

### β₁-Adrenergic Receptor Polymorphisms Effect Antihypertensive Treatment

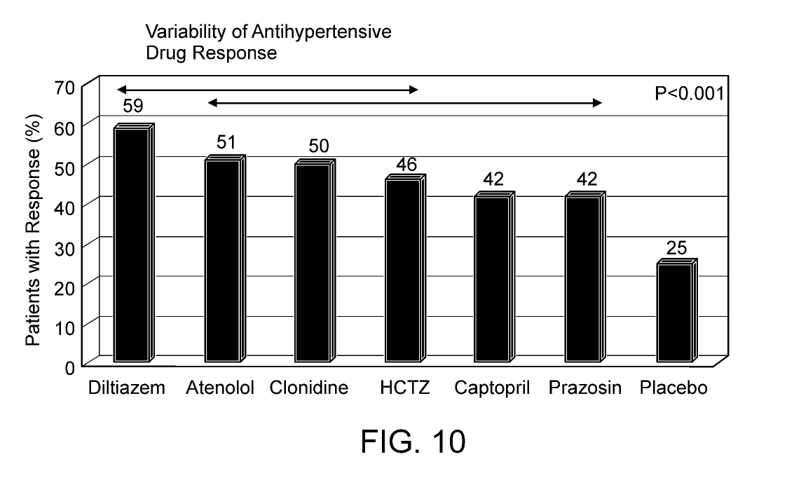

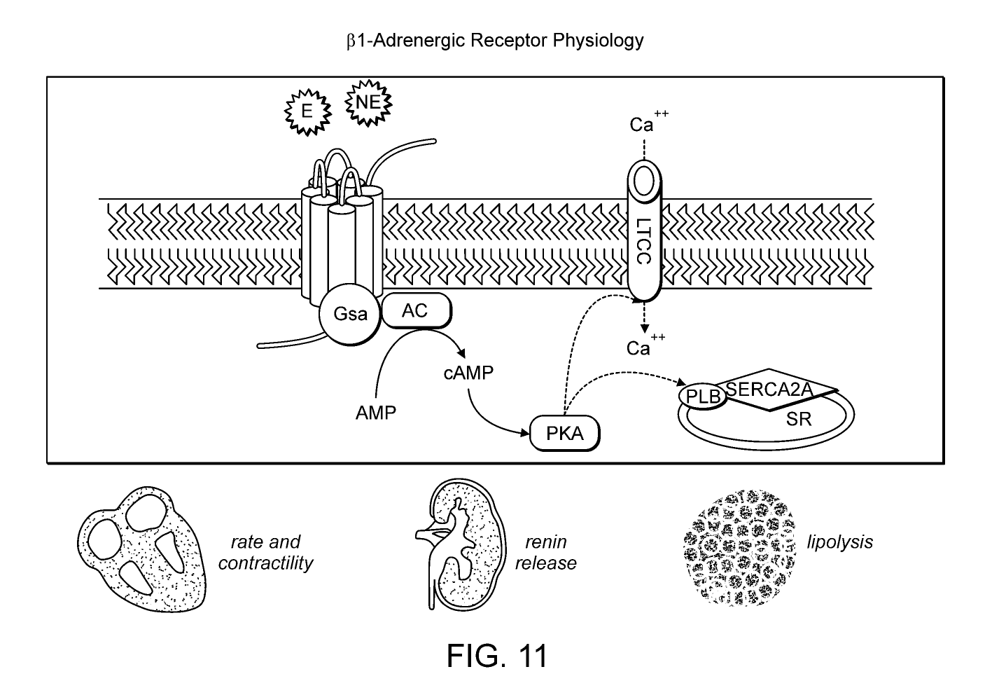

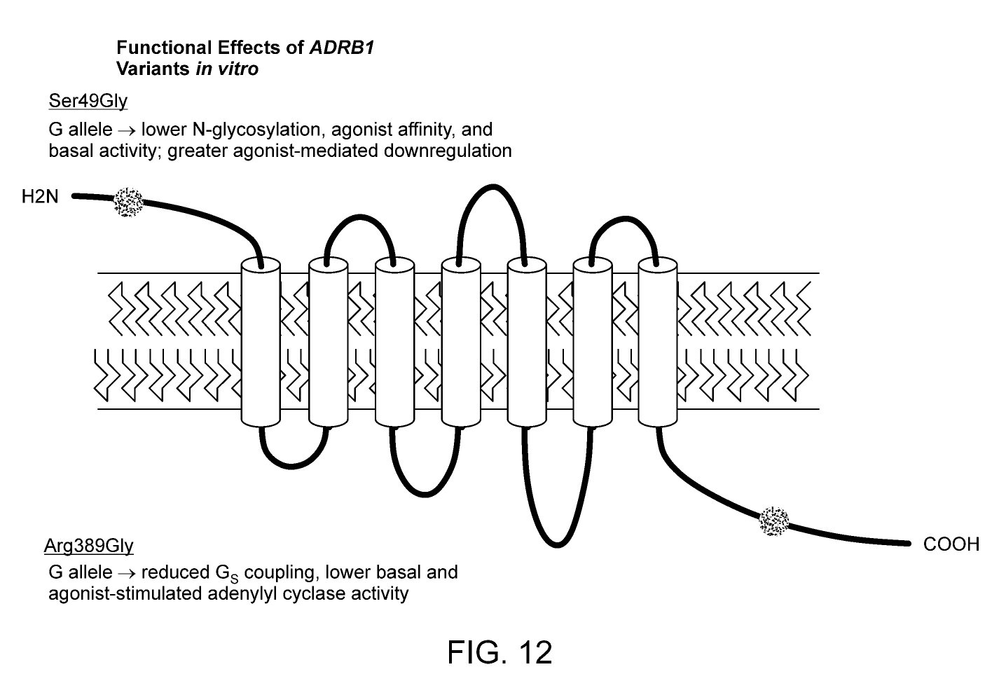

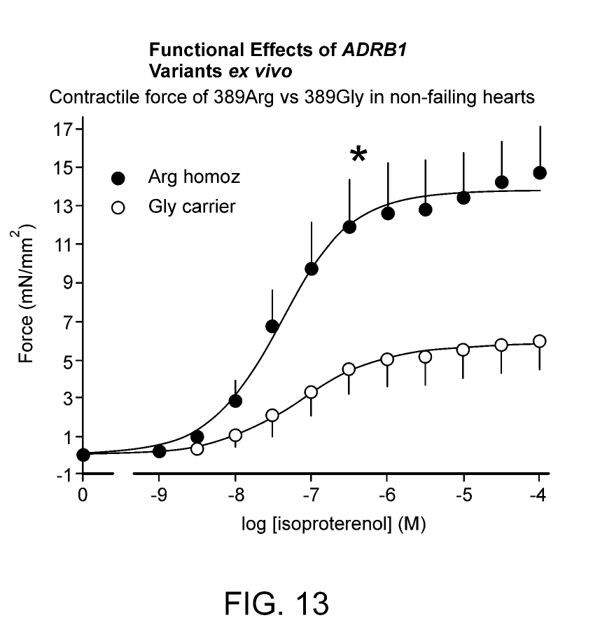

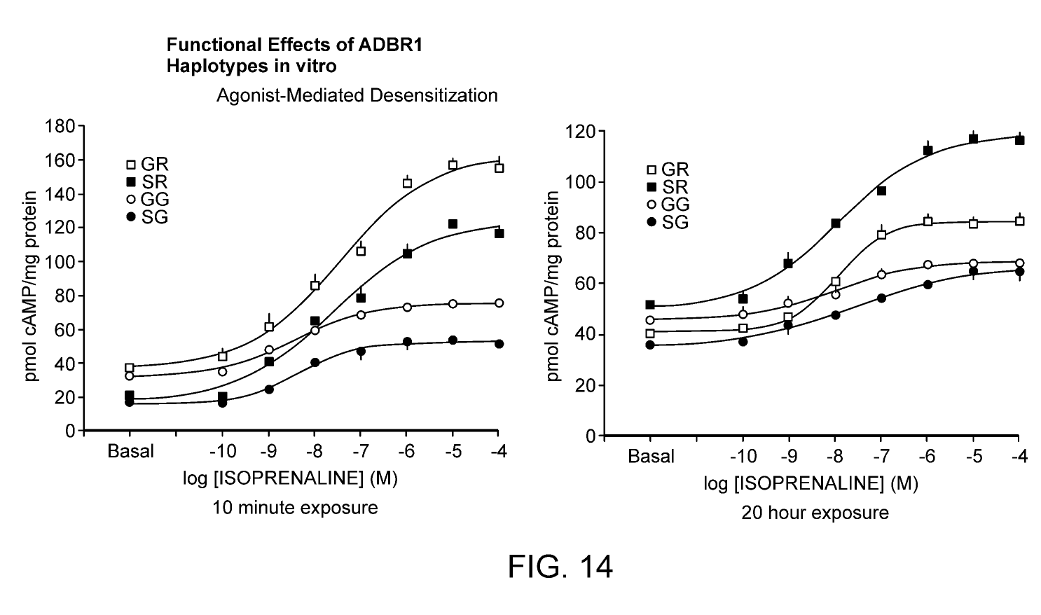

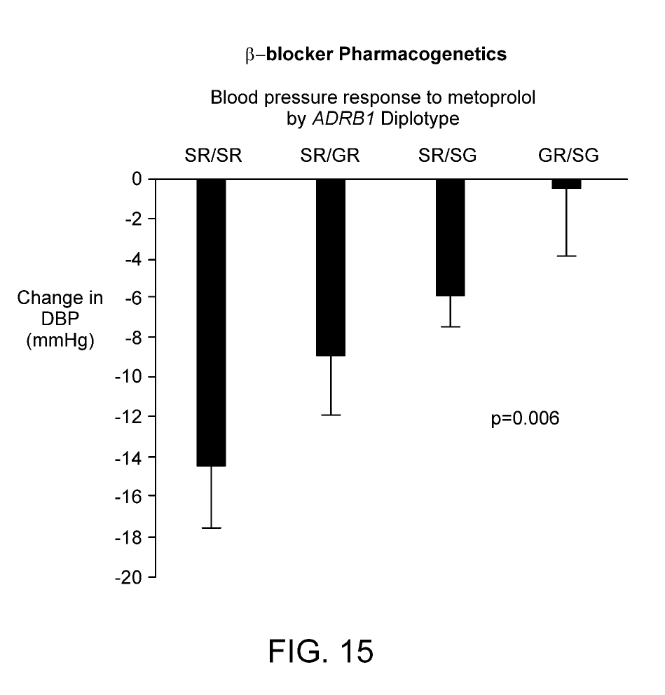

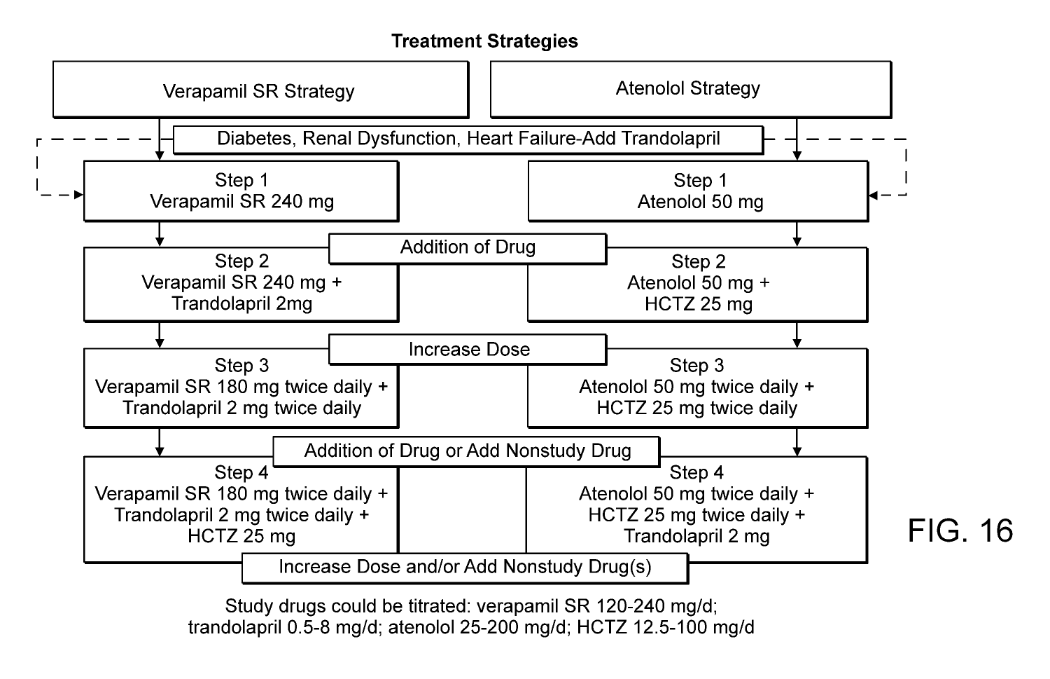

Patients treated with various antihypertensive therapies show variability in their responsiveness (FIG. 10). FIG. 11 shows the role of the β1-adrenergic receptor in physiology. Common polymorphisms in the β₁-adrenergic receptor gene (ADRB1) are associated with the response to drug therapy (FIGS. 12-14) and in particular to β-blocker therapy (FIG. 15). The aim of this study was to evaluate the impact of ADRB1 variants on clinical outcomes in patients with treated hypertension and explore their interaction with antihypertensive drug therapy. Treatment strategies with verapamil SR or atenolol are shown in FIG. 16.

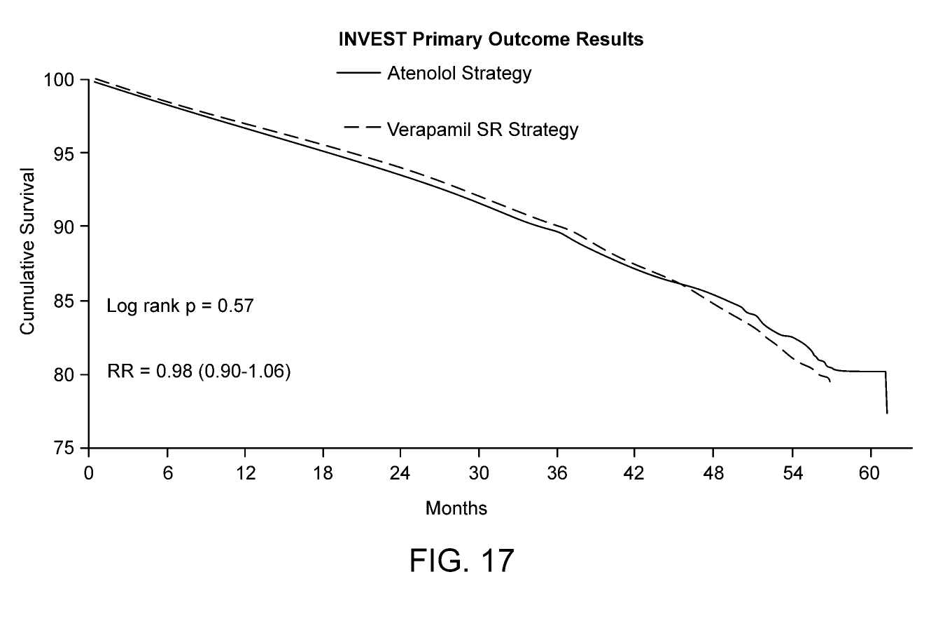

Hypertensive patients with documented coronary artery disease (n=5,979) were randomized to an atenolol- or verapamil SR-based antihypertensive strategy, with hydrochlorothiazide and/or trandolapril added as needed for blood pressure control (FIG. 16). These patients were genotyped for SNPs in ADRB1 (S49G, R389G). Cox regression, adjusted for demographic and clinical factors, was used to model the effects of drug exposure, ADRB1 haplotypes, and their interaction on the primary outcome, which was a composite of all-cause mortality, nonfatal myocardial infarction, and nonfatal stroke (FIG. 17).

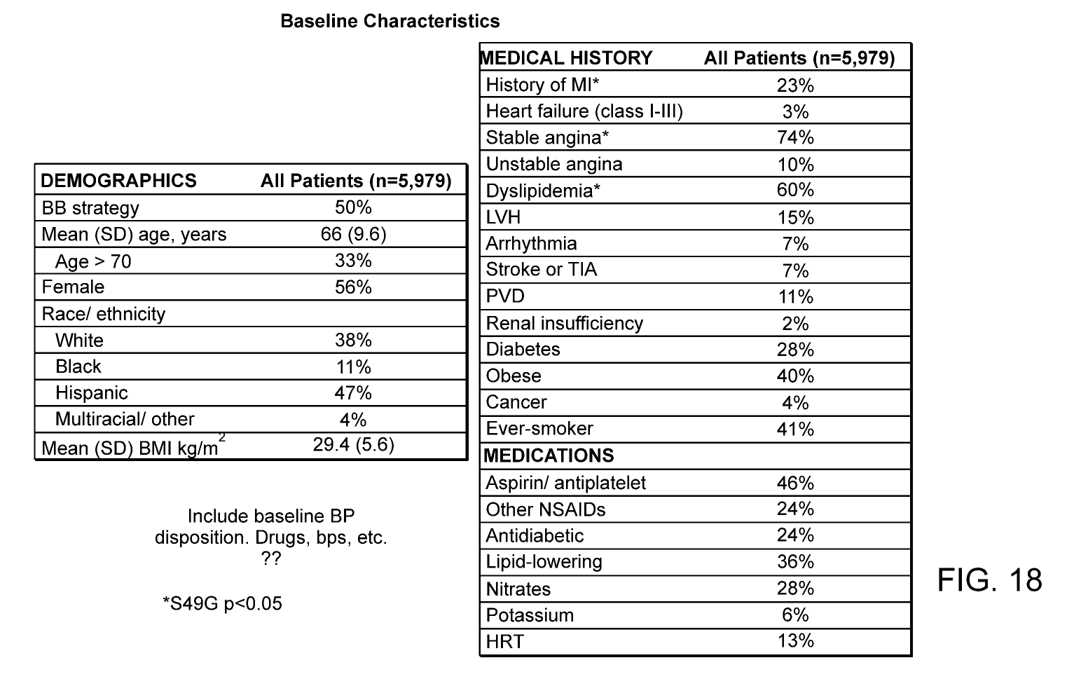

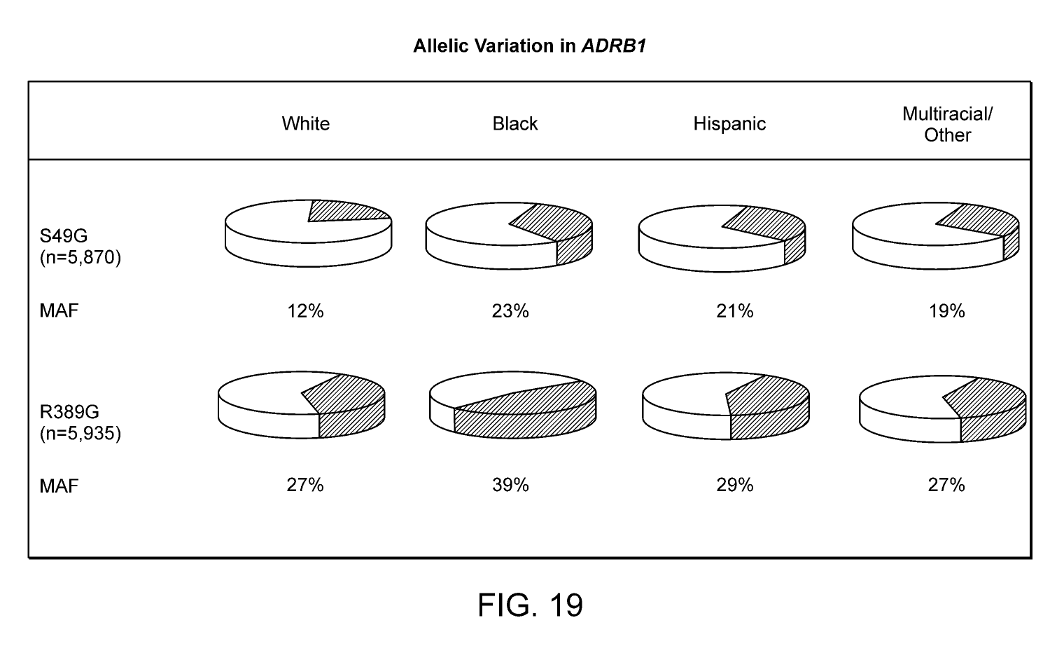

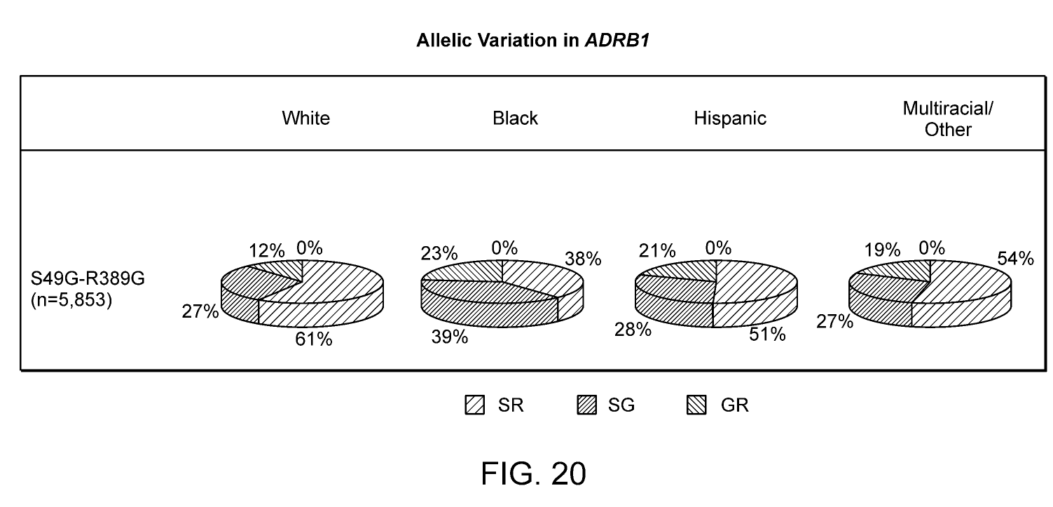

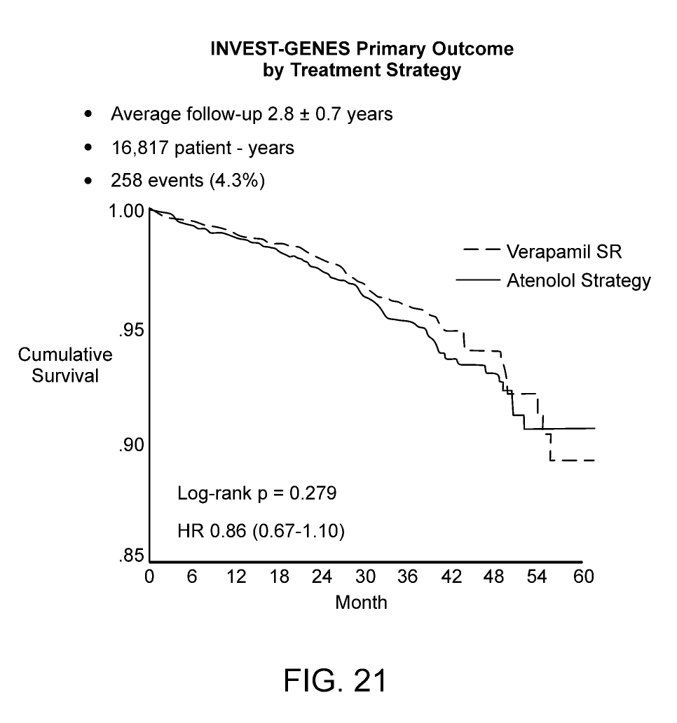

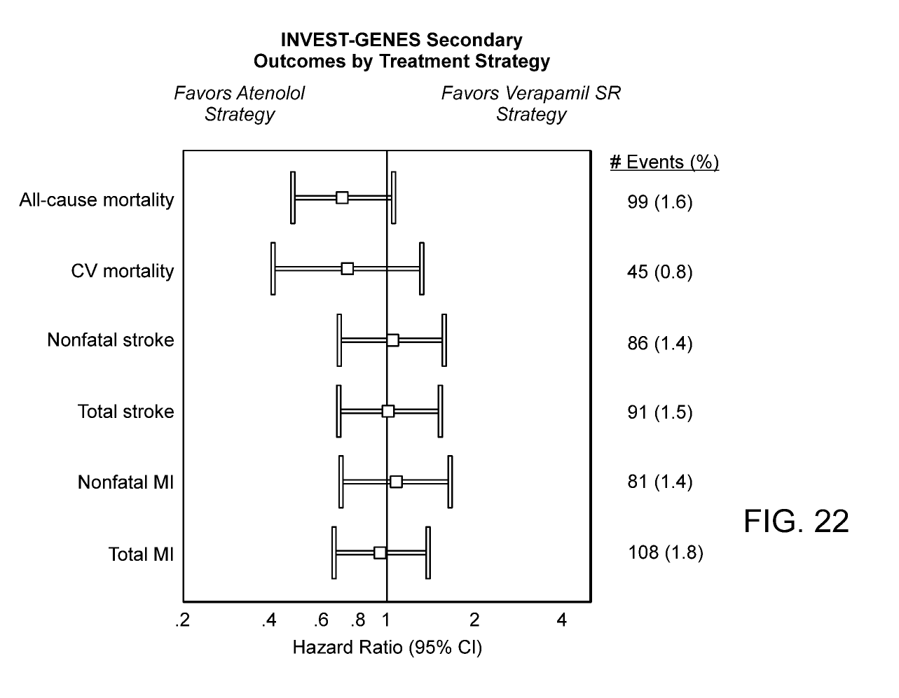

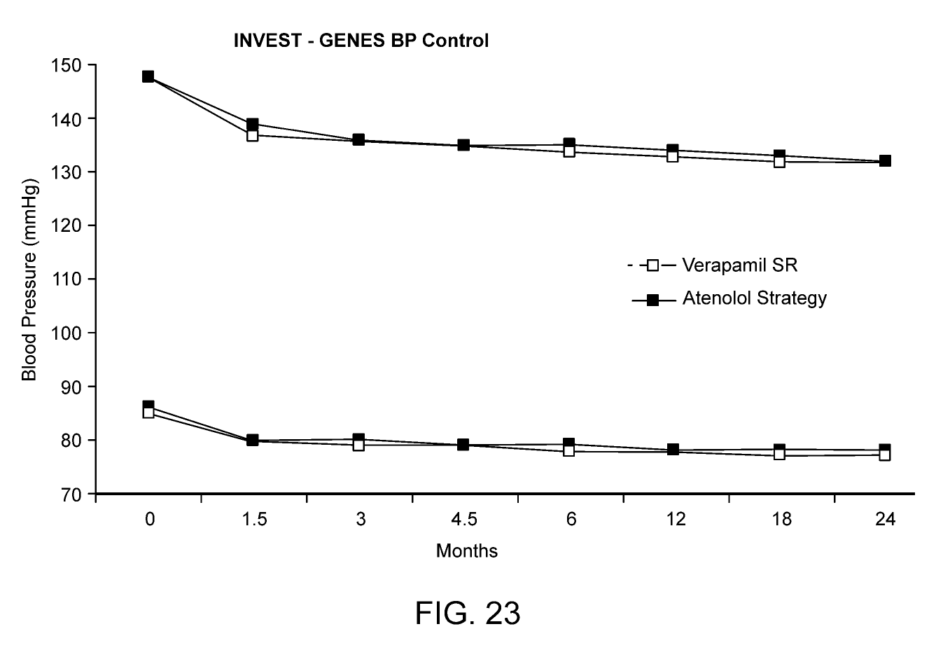

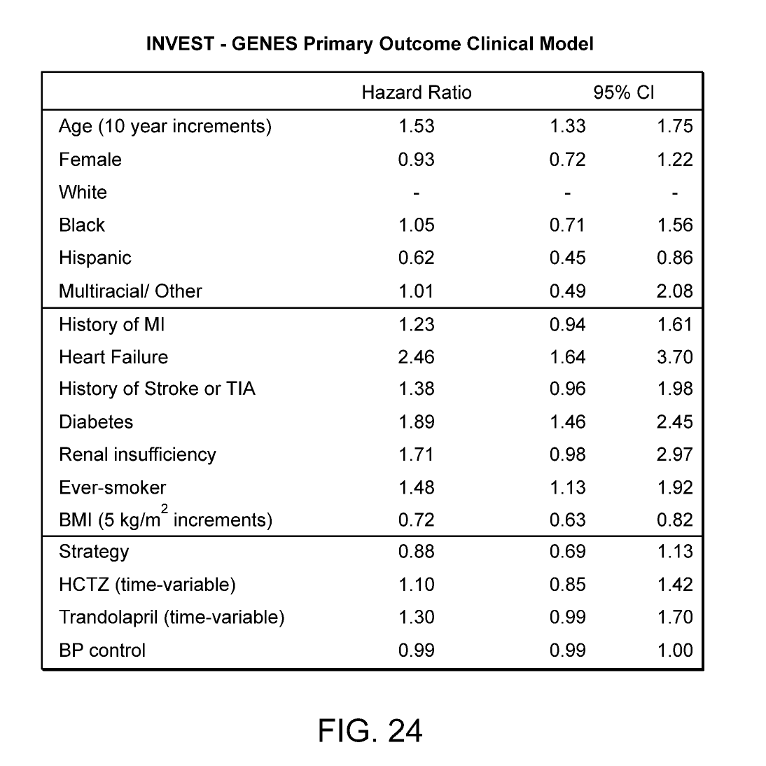

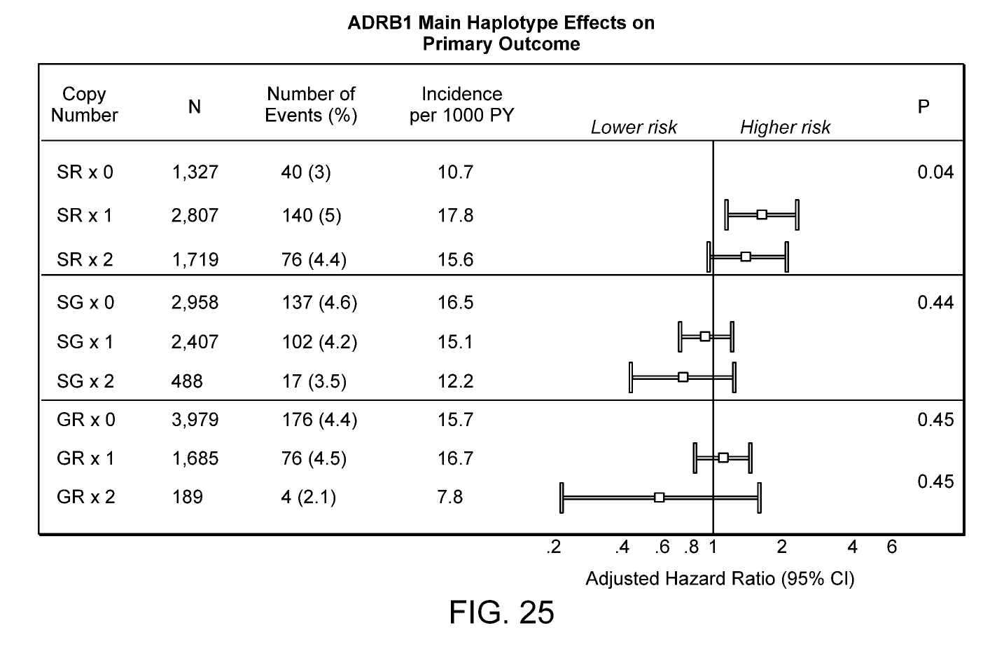

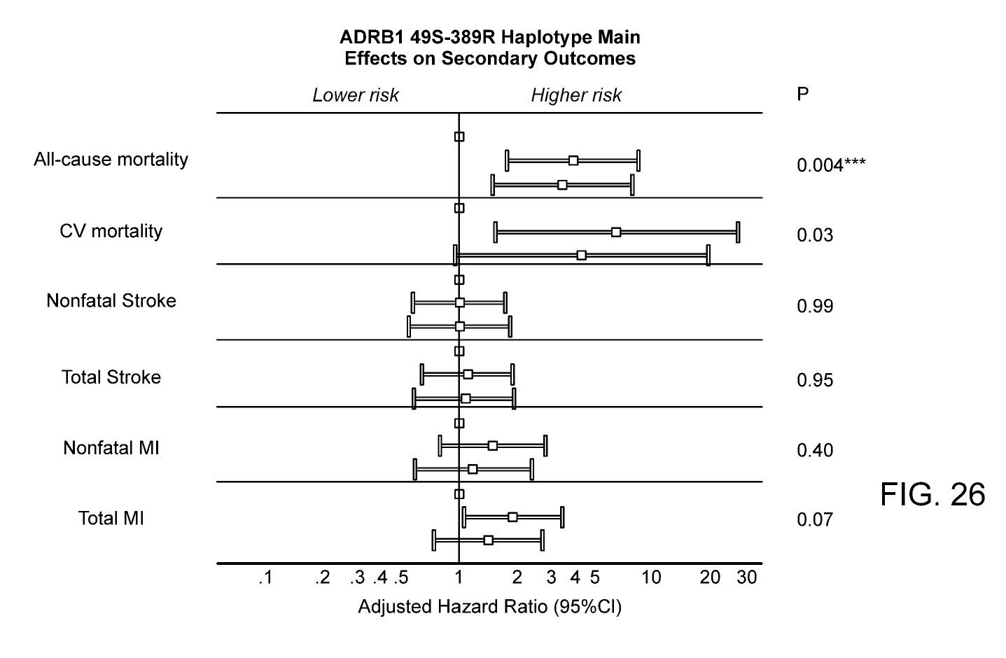

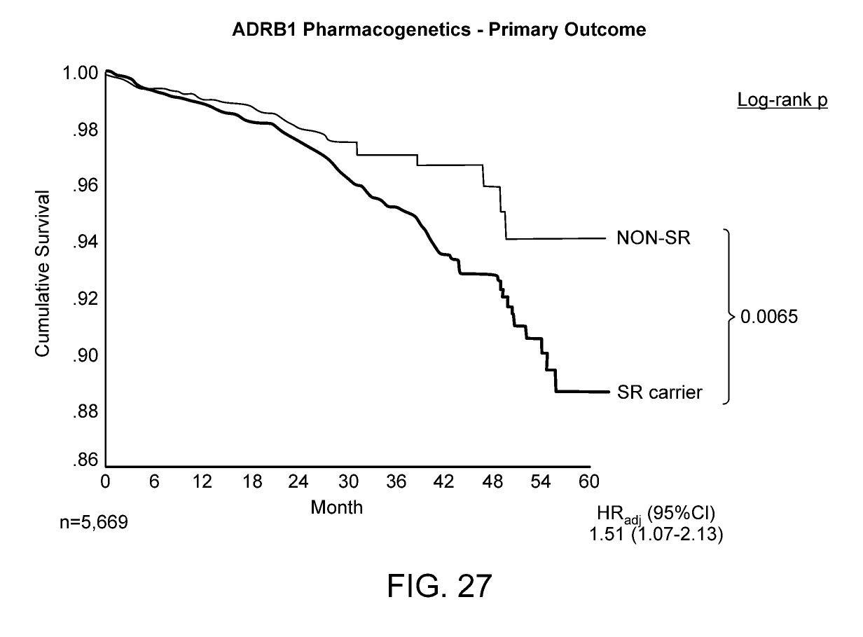

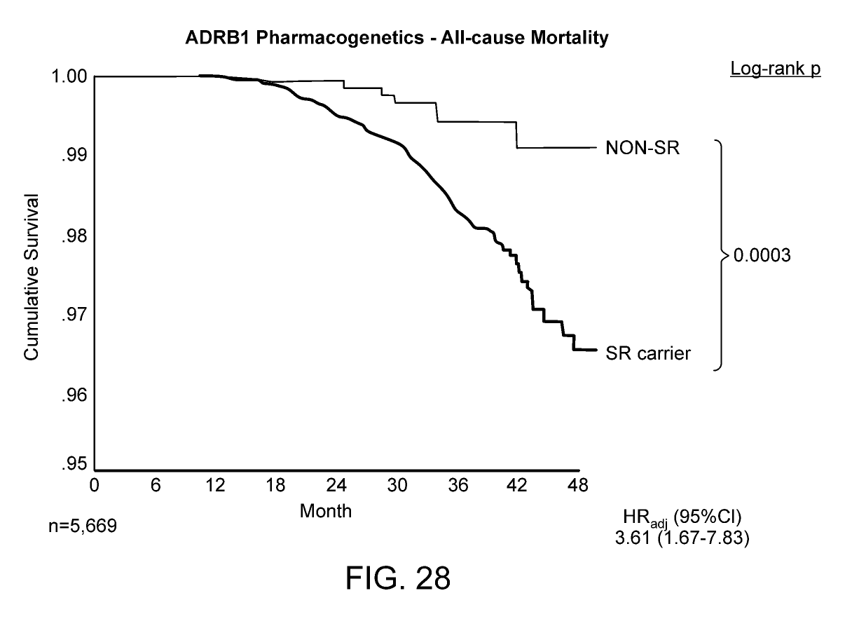

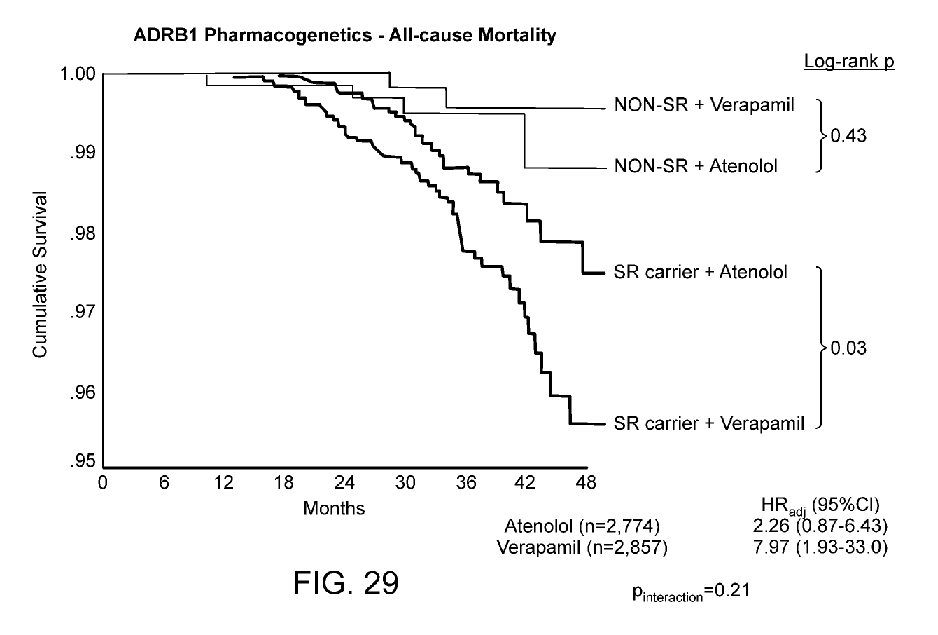

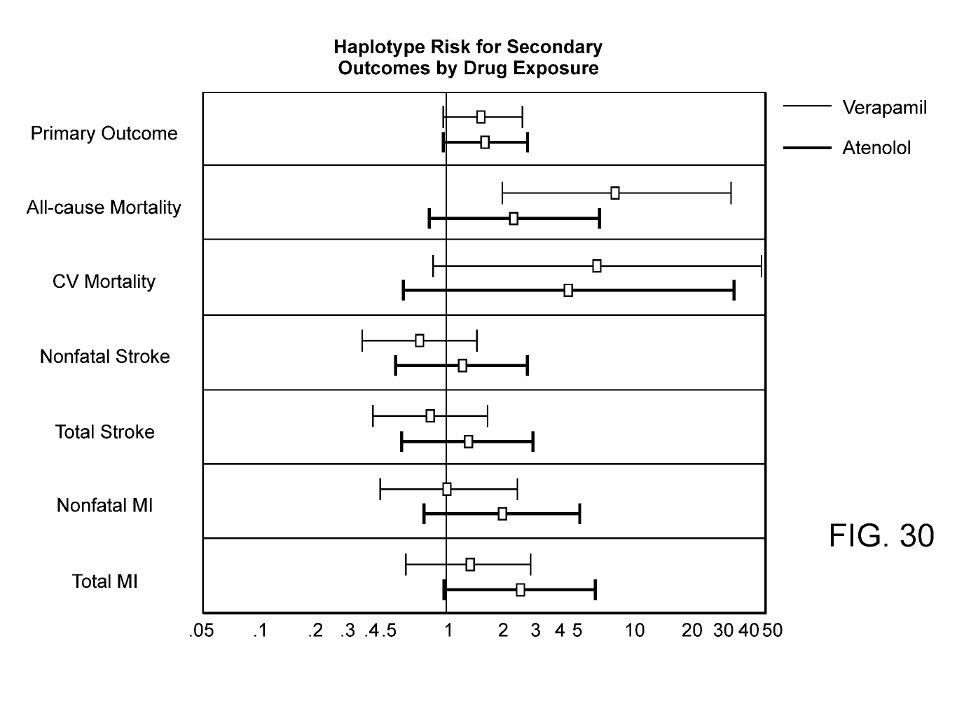

Baseline characteristics of patients in the study population are shown in FIG. 18. Allelic variation as a function of race/ethnicity is shown in FIGS. 19 and 20. After an average follow-up of 2.8 years, 258 patients (4.3%) experienced a primary event (FIG. 21). Secondary outcomes by treatment strategy are shown in FIG. 22. Systolic and diastolic blood pressure control did not differ among patients treated with verapamil and atenolol (FIG. 23). Treatment outcomes did not differ by randomized treatment strategy (adjusted hazard ratio [HR_{adj}] 0.91 [0.72-1.17]; p=0.49) (FIG. 24). ADRB1 variation at codons 49 and 389 was characterized by 3 common haplotypes. Carriers of the 49S-389R haplotype were at greater risk of having a primary outcome event (HR_{adj} 1.57 [1.12-2.20], p=0.02), which was driven by a higher mortality rate in this group (HR_{adj} 3.60 [1.68-7.82]; p=0.001) (FIGS. 25-28). The influence of this haplotype on mortality was less pronounced in patients treated with atenolol (HR_{adj} 2.32 [0.82-6.58]; p=0.11) as compared to verapamil SR (HR_{adj} 8.00 [1.93-33.07]; p=0.004), which indicated a pharmacogenetic relationship (FIGS. 29 and 30).

β₁-AR variants (49S-389R haplotype) are of prognostic importance in hypertensive patients with coronary artery disease. Antihypertensive drug therapy did not significantly alter adverse haplotype effects for the composite outcome, although β-blocker therapy attenuated the adverse effect of this haplotype on mortality.

### Example 4

### β₂-Adrenergic Receptor Polymorphisms

Population-based studies suggest that incident cardiovascular events are associated with polymorphisms in the β₂-adrenergic receptor gene (ADRB2). The aim of this study was to evaluate the impact of ADRB2 variants on clinical outcomes in patients with treated hypertension and explore their interaction with antihypertensive drug therapy in the setting of a clinical trial.

In INVEST, hypertensive patients with documented coronary artery disease were randomized to an atenolol- or verapamil SR-based antihypertensive strategy, with hydrochlorothiazide and/or trandolapril added as needed for blood pressure control. A subset of 5,979 genetic substudy participants were genotyped for polymorphisms in ADRB2 (46 A>G [R16G], 79 C>G [Q27E], 523 C>A). Cox regression, adjusted for demographic and clinical factors, was used to model the effects of ADRB2 haplotypes and their interaction with antihypertensive drugs on the primary outcome (composite of all-cause mortality, nonfatal myocardial infarction, and nonfatal stroke).

After an average follow-up of 2.8 years, 258 patients (4.3%) experienced a primary event. Treatment outcomes did not differ by randomized antihypertensive strategy. Carriers of the 46G-79C-523C haplotype were at greater risk of having a primary outcome event (adjusted hazard ratio 2.05 [1.39-3.00]; p=0.0003). Neither atenolol nor verapamil SR modified the adverse effect of this haplotype. Further pharmacogenetic analysis for other common haplotypes revealed that outcomes in atenolol- and verapamil SR-exposed patients varied as a function of the 46G-79G-523C haplotype (pᵢₙₜₑᵣₐₜᵢₒₙ=0.006).

TABLE 8

| | | | | | 95% |
|---|---|---|---|---|---|
| | | Events/1000 Patient-Years | | Adjusted | Confidence |
| | | Verapamil SR | Atenolol | Hazard Ratio | Interval |
| All | | 16.4 | 13.6 | 0.85 | 0.66-1.10 |
| Patients | | | | | |
| GGC | 0 | 18.0 | 10.6 | 0.59 | 0.40-0.86 |
| Copies | 1 | 14.4 | 15.1 | 1.07 | 0.71-1.60 |
| | 2 | 17.1 | 23.5 | 1.50 | 0.73-3.05 |

Hypertensive patients with stable coronary artery disease having the ADRB2 46G-79C-523C haplotype have an increased risk of adverse cardiovascular events relative to patients that do not carry this variation. The significant gene-drug interaction discovered in these studies indicates that ADRB2 variants may also modify the outcomes associated with antihypertensive therapy. Non-carriers of 46G-79G-523C may have better outcomes with atenolol, while verapamil SR may be preferred in 46G-79G-523C homozygotes.

### Example 5

### Arachidonate 5-Lipoxygenase Pathway Gene Polymorphisms Confer Race-Dependant Risk of Cardiovascular

Recent studies suggest a role for the arachidonic acid 5-lipoxygenase pathway in cardiovascular disease. Relationships between single nucleotide polymorphisms (SNP) and haplotypes of arachidonate 5-lipoxygenase pathway genes and cardiovascular events were tested. In INVEST, hypertensive subjects with coronary artery disease were randomized to either atenolol or verapamil-SR-based treatment strategies with trandolopril and hydrochlorothiazide added as needed for blood pressure control. A nested case-control study within INVEST was conducted, where subjects who experienced a primary outcome (all-cause death, nonfatal myocardial infarction or nonfatal stroke, n=256) were frequency-matched based on age, race and gender to event-free control subjects. A haplotype of ALOX5AP and SNPs was genotyped in both LTC4S and LTA4H, encoding 5-lipoxygenase activating protein, leukotriene C4 synthase and leukotriene A4 hydrolase respectively. The effects of these variants on the primary outcome using the X² test and logistic regression was analyzed.

The overall event risk was not affected for any variant tested. When tested within racial groups, variants in ALOX5AP and LTA4H showed both different genotype frequencies and significant associations with the primary outcome within a single racial group. The haplotype GTC in ALOX5AP was associated with decreased risk of events in Caucasians, in whom the haplotype is most frequent. For LTA4H, two SNPs were positively associated with event risk in African Americans. Further, the homozygous variant genotype frequency of each of these SNPs was lower in African Americans than in the entire group. No associations were seen with the LTC4S SNP.

| | | | | 95% |
|---|---|---|---|---|
| | | Genotype | Adjusted | Confidence |
| Genotype | Group tested | Frequency | Odds Ratio | Interval |
| ALOX5AP | Overall | 0.13 | 0.544 | 0.316-0.937 |
| GTC 2 copies | Caucasians | 0.18 | 0.409 | 0.213-0.788* |
| LTA4H | Overall | 0.32 | 1.023 | 0.742-1.410 |
| rs1978331 AA | AA | 0.06 | 6.567 | 1.806-23.880* |
| LTA4H | Overall | 0.45 | 0.927 | 0.684-1.255 |
| rs2247570 TT | AA | 0.30 | 5.306 | 2.018-13.949* |

AA = African Americans
*statistically significant

Race is a significant factor in determining the risk of cardiovascular events associated with arachidonate 5-lipoxygenase pathway polymorphisms. These results suggest that variants in arachidonate 5-lipoxygenase pathway genes significantly contribute to cardiovascular events in a high cardiovascular risk population.

### Example 6

### LTA4H Variant Confers Drug Therapy-Dependent Reduced Risk of Cardiovascular Events

Recent studies suggest a role for the arachidonic acid 5-lipoxygenase pathway in cardiovascular disease. Pharmacogenetic relationships were tested between a polymorphism of the LTA4H gene which encodes leukotriene A4 hydrolase and cardiovascular events based on drug-treatment. In INVEST, hypertensive subjects with coronary artery disease were randomized to either atenolol or verapamil-SR-based treatment strategies with trandolopril and hydrochlorothiazide added as needed for blood pressure control or other indications. A nested case-control study was conducted within INVEST, where subjects who experienced a primary outcome (all-cause death, nonfatal myocardial infarction or nonfatal stroke) were frequency-matched based on age, race and gender to event-free control subjects. The LTA4H SNP rs2660845 were genotyped and analyzed the effect and interaction of this variant with antihypertensive drug treatment on the primary outcome with the X² test and logistic regression.

There was no difference in the overall event risk between carriers of the LTA4H G-allele and AA homozygotes. Among AA homozygotes atenolol treatment was associated with a decreased risk for events as compared to verapamil SR treatment. Subjects who carried a LTA4H G-allele showed no modification of risk based on antihypertensive treatment.

TABLE 9

| | Total Number | | | |
|---|---|---|---|---|
| | of Events | | | |
| | | Verapamil | Adjusted | 95% Confidence |
| Genotype | Atenolol | SR | Odds Ratio | Interval |
| All subjects | 113 | 137 | 0.82 | 0.60-1.11 |
| (n = 935) | | | | |
| LTA4H | 63 | 60 | 1.20 | 0.79-1.83 |
| G-carriers | | | | |
| (n = 466) | | | | |
| LTA4H AA | 41 | 68 | 0.51 | 0.31-0.81 |
| (n = 469) | | | | |

Carriers of the LTA4H AA genotype (rs2660845) may have better cardiovascular outcomes when treated with atenolol, while carriers of the G-allele may have similar outcomes with either atenolol or verapamil SR. Pharmacologically, this effect may be mediated increased cysteinyl leukotriene production, leading to increased protein kinase C activation and subsequent β-adrenergic receptor modulation.

### Example 7

### Polymorphisms of β₁ and β₂ Adrenergic Receptor Genes (ADRB1, ADRB2) were Associated with Blood Pressure Response

This study tested the hypothesis that polymorphisms of β₁ and β₂ adrenergic receptor genes (ADRB1, ADRB2) were associated with blood pressure response and the number of antihypertensive drugs needed to reach blood pressure control. This was tested in International VErapamil-SR Trandolapril Study Genetic substudy participants randomized to the β-blocker strategy. Five polymorphisms were genotyped by Pyrosequencing in 2,830 patients who received atenolol: ADRB1: Arg389Gly, Ser49Gly, ADBR2: Gly16Arg, Gln27Glu, 523C>A. The ADRB2 haplotype Gly16-Glu27-523C (GEC) was associated with poor blood pressure response to atenolol and the need for more antihypertensive drugs to control blood pressure even after adjusting for covariates. Patients with 0, 1, 2 copies of GEC had covariate-adjusted systolic blood pressure reduction of −11.4, −7.9, −6.7 mmHg, respectively (p=0.028). Patients with 1 copy (odds ratio 1.22, 95% confidence interval [1.03, 1.44], p=0.022) and 2 copies (odds ratio 1.44 [1.08, 1.91], p=0.01) of ADRB2 haplotype GEC were more likely to require more drugs to control blood pressure than patients who are not carriers of GEC. Patients with higher baseline systolic blood pressure, diabetes, history of heart failure were also more likely to require more drugs to control blood pressure than those without these risk factors. These data indicated that ADRB2 haplotype GEC is associated with a poorer blood pressure response to atenolol therapy in older hypertensives with coronary artery disease, thus necessitating a greater number of drugs needed to attain blood pressure control.

### Baseline Characteristics and Drug Exposure

A total of 2,830 (94.0% of 3,012) patients in the β-blocker treatment strategy actually received atenolol during the study. Baseline characteristics, blood pressure and antihypertensive medications use of the patients in the β-blocker strategy are summarized in Table 10.

TABLE 10
Baseline characteristics, antihypertensive medications and intermediate
outcomes of Patients in atenolol-based Beta blocker strategy*.

| | Patients on | |
|---|---|---|
| | Atenolol | |
| | (N = 2,830) | |
| Demographic | | |
| Age, mean (SD), y | 66.0 | (9.7) |
| >70 y | 956 | (33.8) |
| Women | 1565 | (55.3) |
| Ethnicity | | |
| White | 1058 | (37.4) |
| Black | 312 | (11.0) |
| Hispanic | 1346 | (47.6) |
| Other/Multiracial | 114 | (4.0) |
| BMI, mean (SD), kg/m² | 29.4 | (5.6) |
| Baseline condition | | |
| Heart rate, (SD), beats/min | 74.7 | (9.6) |
| Blood pressure, mean (SD), mmHg | | |
| Systolic | 148.3 | (18.1) |
| Diastolic | 85.8 | (10.7) |
| No. (%) with blood pressure in control at study entry | | |
| Systolic | 784 | (27.7) |
| Diastolic | 1654 | (58.4) |
| Both | 678 | (24.0) |
| Added study drugs | | |
| HCTZ | 2099 | (74.2) |
| Trandolapril | 1967 | (67.5) |
| No. of strategy drugs at end of follow-up, mean (SD) | 1.9 | (1.0) |
| Total No. of antihypertensive drugs at end of | 2.7 | (1.3) |
| follow-up, mean (SD) | | |
| Intermediate outcomes | | |
| Percent of visits with blood pressure in | 59.4 | (27.5) |
| control‡, mean (SD) | | |
| SBP at 12 months, mean (SD), mmHg§ | 134.0 | (17.1) |
| DBP at 12 months, mean (SD), mmHg§ | 78.3 | (9.5) |
| BP in control at 12 months§, No. (%) | 1725 | (66.0) |
| SBP at 24 months, mean (SD), mmHg§ | 132.3 | (16.2) |
| DBP at 24 months, mean (SD), mmHg§ | 77.7 | (9.2) |
| BP in control at 24 months§, No. (%) | 1695 | (69.5) |

Abbreviations:
BMI: body mass index;
SD: standard deviation;
BP: blood pressure;
HCTZ: hydrochlorothiazide.
*Values expressed as number (percentage) unless otherwise indicated. Percentages may not add to 100 due to rounding.
‡BP in control as SBP < 140 mmHg and diastolic blood pressure (DBP) < 90 mmHg
§Percentage of patients at risk at 12 or 24 months.

Patients included a large proportion of elderly (mean (±SD) age was: 66±9.7 years), diabetic (29%) and female (55%) patients that were ethnically diverse with 47% Hispanics, 38% whites and 11% African Americans/Blacks. The initial atenolol daily dose was 25 mg daily in 353 (12.5%) patients, 50 mg in 2,104 (74.3%) patients and 100 mg in 252 (8.9%) patients. 2,163 (76.4%) patients continued atenolol therapy until the end of the study, with dose titration or addition of other antihypertensive medications as necessary to control blood pressure. At the end of the study, the median atenolol dose was 50 mg, with 188 (8.7%) patients on 25 mg/d, 649 (30.0%) on 50 mg/d, 40 (1.9%) on 75 mg/d, 1,124 (52.0%) on 100 mg/d, 56 (2.6%) on 150 mg/d, 106 (4.9%) patients on 200 mg/d of atenolol. The median duration of atenolol therapy was 2.75 years (interquartile range: 0.78 years), consistent with the median duration of follow-up in the entire INVEST cohort of 2.7 years.

### Genotypes and Haplotypes

2,772 (98%) patients were successfully genotyped for ADRB1 Arg389Gly, Ser49Gly, ADRB2 Gly16Arg, Gln27Glu and 523 C>A. The allele frequencies of the five SNPs in each race/ethnicity group are consistent with literature reports (shown in Table 11).

TABLE 11
ADRB1 and ADRB2 Polymorphism Allele frequencies and Haplotype
Frequencies by Race/ethnicity.

| | Frequency, % | | | | |
|---|---|---|---|---|---|
| | White | Hispanics | Black | Other/Multiracial | |
| | (n = 1,033) | (n = 1,320) | (n = 305) | (n = 114) | P value* |
| ADRB1 | | | | | |
| Ser49Gly | | | | | |
| Ser49 | 87.9 | 78.9 | 75.8 | 79.4 | <0.0001 |
| Gly49 | 12.1 | 21.1 | 24.2 | 20.6 | |
| Arg389Gly | | | | | |
| Arg389 | 71.4 | 71.2 | 61.8 | 71.1 | <0.0001 |
| Gly389 | 28.6 | 28.8 | 38.2 | 28.9 | |
| Haplotype | | | | | |
| H1 (Ser49-Arg389) | 59.5 | 50.4 | 38.0 | 50.4 | <0.0001 |
| H2 (Ser49-Gly389) | 28.4 | 28.5 | 37.8 | 29.0 | |
| H3 (Gly49-Arg389) | 11.9 | 20.9 | 24.2 | 20.6 | |
| H4 (Gly49-Gly389) | 0.2 | 0.2 | 0 | 0 | |
| ADRB2 | | | | | |
| Gly16Arg | | | | | |
| Gly16 | 59.3 | 54.3 | 52.6 | 53.9 | 0.0045 |
| Arg16 | 40.7 | 45.6 | 47.4 | 46.1 | |
| Gln27Glu | | | | | |
| Gln27 | 59.3 | 71.3 | 81.8 | 74.1 | <0.0001 |
| Glu27 | 40.7 | 28.7 | 18.2 | 25.9 | |
| 523C > A | | | | | |
| 523C | 78.8 | 70.5 | 60.2 | 69.3 | <0.0001 |
| 523A | 21.2 | 29.5 | 39.8 | 30.7 | |
| Haplotype | | | | | |
| H1 (Arg16-Gln27-523C) | 37.6 | 42.7 | 43.6 | 41.2 | <0.0001 |
| H2 (Gly16-Glu27-523C) | 35.5 | 24.2 | 13.8 | 24.1 | |
| H3 (Gly16-Gln27-523A) | 16.5 | 23.6 | 34.7 | 25.9 | |
| H4 (Gly16-Glu27-523A) | 3.3 | 3.7 | 2.6 | 0.9 | |
| H5 (Gly16-Gln27-523C) | 4.0 | 2.8 | 1.5 | 3.1 | |
| H6 (Arg16-Gln27-523A) | 1.3 | 2.2 | 2.0 | 3.9 | |
| H7 (Arg16-Glu27-523C) | 1.3 | 0.8 | 1.3 | 0.9 | |
| H8 (Arg16-Glu27-523A) | 0.2 | 0.5 | 0 | 0 | |

*The p values for the χ² test of the minor allele frequency and haplotype frequency by race/ethnicity.

All five SNPs were common SNPs with minor allele frequencies >10% in each race/ethnicity group. The minor allele frequencies were significantly different between race/ethnicity groups with the frequencies in Hispanics intermediate between Caucasians and African Americans for all SNPs (Table 11). The allele frequencies did not deviate from Hardy Weinberg Equilibrium except Gly16Arg in the Hispanic population. This deviation might be explained by the highly admixed population structure of our Hispanic patients. ADRB1 Ser49Gly and Arg389Gly are in strong linkage disequilibrium (D′=0.83 in Caucasians, 0.93 in Hispanics and 1.0 in African Americans and other/multiracial). As expected, less than 1% of the patients carry haplotype Gly49Gly389 (Table 11). The three variants in ADRB2 are also in linkage disequilibrium with pairwise D′ of 0.60-0.91. The structure and frequencies of the ADRB1 and ADRB2 haplotypes in Caucasians, African Americans and Hispanics are shown in Table 11. The haplotype frequencies were also significantly different among the three populations.

### ADRB1, ADRB2 Polymorphisms and Blood Pressure Response to Atenolol

In the 619 patients whose blood pressure change could be reliably attributed to atenolol, the mean±SD pre-treatment blood pressure was 150.8±16.4/86.6±10.5 mmHg and the mean blood pressure after 6 weeks of treatment was 139.7±18.3/80.4±9.9 mmHg, with the mean reduction in blood pressure being: −11.1±19.5/−6.2±10.3 mmHg.

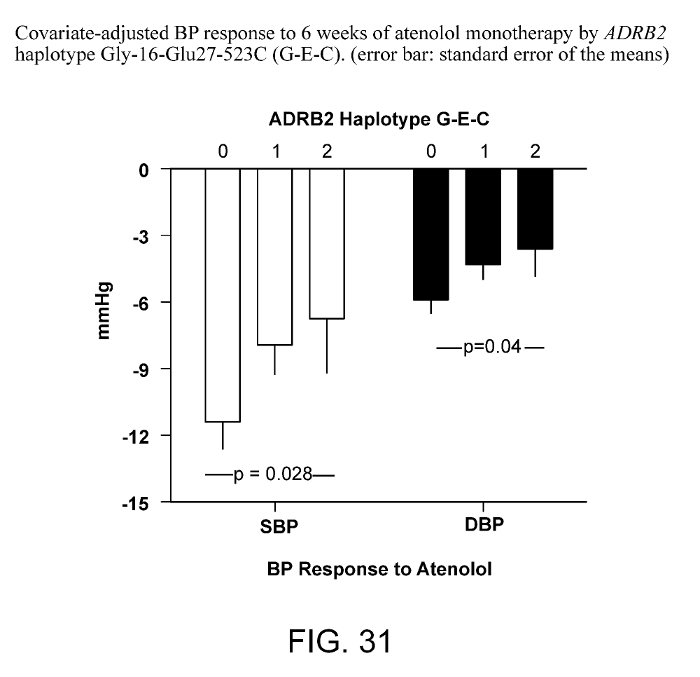

Haplotype analysis showed that ADRB2 haplotype H₂: Gly16-Glu27-C523 (GEC) was significantly associated with systolic blood pressure and diastolic blood pressure reduction. 39% of the patients were heterozygous (1 copy) and 9% were homozygous (2 copies) for this haplotype. Patients with 0 copies of this haplotype had adjusted systolic blood pressure change of −11.4 mmHg, 1 copy: −7.9 mmHg and 2 copies: −6.7 mmHg (p=0.028) (FIG. 31), consistent with a gene dose effect. There were similar patterns with the diastolic blood pressure response (covariate-adjusted diastolic blood pressure reduction p=0.04). Diplotype analysis had less predictive power than the haplotype analysis. ADRB1 haplotypes were not significantly associated with blood pressure response to atenolol (p values >0.2).

Individual ADRB2 SNP analysis showed that the treatment systolic blood pressure was significantly associated with ADRB2 Gln27Glu (p=0.048) and 523 C>A (p=0.021) after adjusting for pre-specified covariates such as pre-treatment systolic blood pressure, age, gender and race/ethnicity (Table 12).

TABLE 12
Blood pressure response by individual ADRB2 genotype.

| | | | Covariate-Adjusted Decline in blood | | | p |
|---|---|---|---|---|---|---|
| Gene | Polymorphism | BP | pressure (mmHg) by Genotype | | | genotype |
| ADRB2 | Gly16Arg | | GlyGly | GlyArg | ArgArg | |
| | | Systolic | −9.0 | −10.9 | −8.0 | 0.23 |
| | | Diastolic | −4.2 | −5.9 | −4.9 | 0.08 |
| | Gln27Glu | | GlnGln | GlnGlu | GluGlu | |
| | | Systolic | −11.2 | −8.8 | −6.0 | 0.048* |
| | | Diastolic | −5.9 | −4.8 | −3.4 | 0.07 |
| | C523A | | CC | CA | AA | |
| | | Systolic | −8.2 | −9.9 | −15.2 | 0.021* |
| | | Diastolic | −5.1 | −4.5 | −7.4 | 0.09 |

Patients who are homozygous for Gln27 or 523A had the greatest reduction in systolic blood pressure. Arg16Gly, Gln27Glu and 523C>A were also marginally associated with diastolic blood pressure response, with p values of between 0.07-0.09 (Table 12).

ADRB2 Haplotype was Associated with Number of Antihypertensive Drugs Needed to Reach Blood Pressure Control

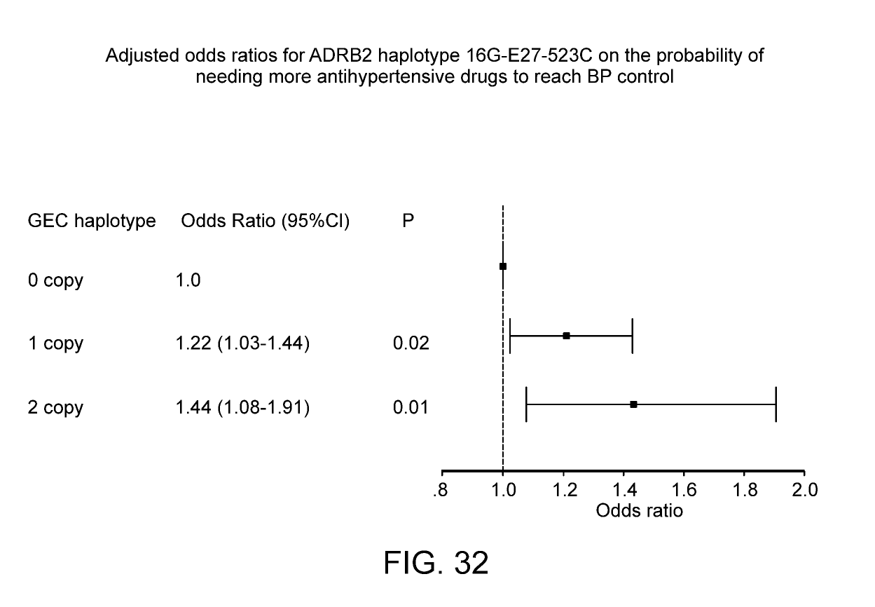

Of the 2,772 patients with complete genotype data, 2,078 (75.0%) patients achieved sustained blood pressure control (<140/90 mmHg, with at least 50% of the subsequent study visits also controlled) (sustained blood pressure control). Consistent with blood pressure response in the smaller cohort, ADRB2 haplotype H₂: Gly16-Glu27-523C (GEC) was associated with needing more antihypertensive drugs to control blood pressure. The haplotype frequencies of GEC were higher in patients who required greater number of antihypertensive drugs to control blood pressure (p for trend=0.02). The unadjusted odds ratio [95% confidence interval] of requiring more drugs for patients with 1 copy and 2 copies vs. 0 copies of this haplotype was 1.19 [1.01, 1.41] (p=0.038) and 1.49 [1.13, 1.97] (p=0.005), respectively. After adjusting for risk factors found associated with requiring more drugs to control blood pressure such as higher baseline systolic blood pressure, diabetes, history of heart failure, peripheral vascular disease, left ventricular hypertrophy, black race, body mass index >30, patients with 1 copy of GEC had 22% higher odds of requiring more antihypertensives to control blood pressure (1.22, [1.03, 1.44], p=0.022) than patients with 0 copies of this haplotype; patient with 2 copies were 44% more likely to require more drugs to control blood pressure (1.44 [1.08, 1.91], p=0.01) (FIG. 32). Subgroup analysis revealed similar associations in Caucasians and Hispanics, but the association was less evident in Blacks. This inconsistency is probably due to lack of power because of the small number of black patients who carry this haplotype. ADRB1 haplotypes were not associated with number of antihypertensive drugs to control blood pressure (p values >0.19).

When treating number of antihypertensive drugs as a binary variable, 844 (40% of the patients who achieved blood pressure control) patients needed three or more drugs to achieve sustained blood pressure control. After adjusting for multiple variables mentioned above, patients with 1 copy and 2 copies of ADRB2 haplotype GEC is 27% and 46% more likely to require 3 or more drugs to achieve sustained blood pressure control compared to patients with 0 copies of this haplotype, with odds ratios of 1.27 [1.02, 1.52] (p=0.03) and 1.46 [1.06, 2.03] (p=0.02), respectively.

### Sensitivity Analysis

The association between ADRB2 haplotype GEC and number of antihypertensives needed to control blood pressure was also assessed in the 619 blood pressure response study patients. 494 (79.8%) of these patients achieved sustained blood pressure control. Patients who are carriers of this haplotype responded poorly to atenolol in the first 6 weeks of treatment and also had 87% higher odds of requiring more antihypertensive drugs to control blood pressure after controlling for multiple confounding factors (1.87 [1.34, 2.61], p=0.0003).

For an additional sensitivity analysis, all the patients in the BB arm who received atenolol were included, including those patients who did not achieve sustained blood pressure control during the study, using the number of antihypertensive drugs taking at the last visit plus one as their number of drugs. A similar result was obtained, with odds ratios for requiring more antihypertensives to control blood pressure of 1.17 [1.02, 1.36] and 1.31 [1.02, 1.67] for patients with 1 copy and 2 copies vs. 0 copies of ADRB2 haplotype GEC, respectively.

As a final sensitivity analysis, the definition of blood pressure control for patients with co-morbid conditions was used according to the JNC VI guidelines (Arch Intern Med. 1997; 157:2413-2446), the then current guideline of INVEST, i.e. <130/85 mmHg for patients with diabetes and renal insufficiency and <140/90 mmHg for others. The association of ADRB2 haplotype GEC and number of antihypertensive drugs needed to control blood pressure remained the same.

These data suggest that carriers of ADRB2 haplotype GEC responded poorly to atenolol, as assessed by change in BP. Consistent with this finding, patients carrying this haplotype were more likely to require multiple antihypertensive drugs to control blood pressure than noncarriers.

Results in Example 7 were obtained using the following materials and methods.

### INternational VErapamil-SR Trandoparil Study (INVEST)

The INVEST rationale, design, inclusion and exclusion criteria, treatment strategies and main results have been published in detail elsewhere (Pepine et al., J Am Coll Cardiol. 1998; 32:1228-1237; Pepine et al., Jama. 2003; 290:2805-2816). Briefly, after an extensive cardiovascular history and physical examination, patients were randomly assigned to either verapamil SR-trandolapril or atenolol-hydrochlorothiazide (HCTZ) based antihypertensive strategy. Patients were evaluated every 6 weeks for the first 6 months and then biannually for at least 2 years, to assess blood pressure and adverse outcomes. The detailed blood pressure measurement method was published previously (Pepine et al., J Am Coll Cardiol. 1998; 32:1228-1237; Cooper-DeHoff et al., Clin Cardiol. 2004; 27:571-576). The INVEST online data system provided detailed tracking of study medication prescriptions (Cooper-DeHoff et al., Clin Cardiol. 2001; 24:V14-16). Between 1997 and 2003, 61,835 patient-years follow-up were accumulated and each strategy provided excellent blood pressure control (>70% of patients achieved blood pressure <140/90 mmHg) without differences in blood pressure between the strategies. The strategies were equally effective in preventing the composite primary outcome of all-cause death, nonfatal myocardial infarction (MI), or nonfatal stroke. The protocol was conducted in accordance with principles outlined in the Declaration of Helsinki, and institutional review boards and ethics committees at participating sites approved the protocol and patients provided written informed consent. Treatment for blood pressure and other medical care were defined per the then current sixth report of the Joint National Committee on Prevention, Detection, Evaluation, and Treatment of High Blood Pressure (JNC VI), (Arch Intern Med. 1997; 157:2413-2446) INVEST Clinical Trial Registration Identifier: NCT00133692; URL: http://clinicaltrials.gov/ct/gui/show/NCT00133692?order=5

### INVEST GENetic Substudy (INVEST GENES)

Genomic DNA samples were collected from 5,979 patients from 184 sites in the mainland United States and Puerto Rico. 3,012 of these patients were randomly assigned to the atenolol-based β-blocker strategy. If patients did not receive target blood pressure, HCTZ was given at step 2 with the rationale to maximize use of the combination of β-blocker and diuretic in this treatment strategy. Doses were increased in step 3 and trandolapril was added in step 4. Additional non-study antihypertensive drugs, except calcium channel blocker, could be added when needed to reach blood pressure targets defined by JNC VI guidelines (Arch Intern Med. 1997; 157:2413-2446).

To address the influence of β adrenergic receptor gene polymorphisms on the blood pressure response to atenolol, 619 INVEST-GENES patients were studied. These patients either entered the study untreated and then were given atenolol monontherapy or were on “stable background therapy” where addition of atenolol was the only change to their anti-hypertensive medication regimen. These patients formed the analysis group for blood pressure response as they are the only patients in whom changes in blood pressures could reliably be attributed to atenolol. The mean of two blood pressure measurements by an oscillometric device or by sphygmomanometry (at lease 5 min apart, with appropriate-sized cuff) was used for the blood pressure at each visit. Blood pressure change from baseline to visit 2 (6-week visit) was evaluated in these patients.

The number of study and nonstudy antihypertensive drugs needed to reach sustained blood pressure control was determined for each patient who received atenolol and had their blood pressure controlled. blood pressure control was defined as achieving, then maintaining a blood pressure <140/90 in at least 50% of subsequent study visits (sustained blood pressure control). Number of drugs needed to reach blood pressure control was used as an indicator of how difficult it was to control the individual's BP, the presumption being that subjects with a ‘β-blocker response’ genotype would respond well to atenolol and would require fewer titration steps (hence, fewer medications) to control BP. All subjects who received atenolol (n=2,830) were included in this analysis.

### Genotyping

Genomic DNA was collected using the buccal cells from mouthwash samples according to previously described methods (Andrisin et al., Pharmacotherapy. 2002; 22:954-960). Genotyping for the ADRB1 Arg389Gly, Ser49Gly, ADRB2 Gly16Arg, Gln27Glu, C523A polymorphisms was performed by polymerase chain reaction (PCR) followed by pyrosequencing (Langaee et al., Mutat Res. 2005; 573:96-102; Pyrosequencing, Uppsala, Sweden) using a PSQ HS96A SNP reagent kit according to the manufacturer's protocol (Biotage AB, Uppsala, Sweden). The PCR and the sequencing primers used for the pyrosequencing genotyping assays for Arg389Gly, Ser49Gly, Gly16Arg and Gln27Glu are described by Shin et al, Am J Cardiol January 2007, in press). The primers used for 523C>A were: 5′-GGA TCG CTA CTT TGC CAT TAC-3′ (PCR-forward), 5′-bio-GGC ATA GGC TTG GTT CGT G-3′ (PCR-reverse) and Sequencing primer: 5′-CAT TCA GAT GCA CTG GT-3′ (Forward). PCR mixture (12.5 μL) consisted of 6.25 μL HotStarTaq® Master Mix Kit (Qiagen Inc, Valencia, Calif.), 1 μL PCR primers (10 pmol/μL), 0.75 μL of dimethyl sulfoxide, 1.25 μL of H₂O, and 40 ng of DNA. PCR was performed under the following conditions: 95° C. for 15 min; 40 cycles consisting of denaturation at 95° C. for 30 sec, annealing at 57° C. for 40 sec and extension at 72° C. for 30 sec; and final extension for 7 min. Pyrosequencing was performed under standard conditions for sequence determination and allele designation and data were captured with PSQ HS96A SNP software.

### Statistical Analysis

Departure of the genotype frequencies from Hardy-Weinberg Equilibrium within each race/ethnicity group was tested using chi-squared test with one degree of freedom. HAP software (using imperfect phylogeny methods coupled with maximum likelihood model) was used to construct haplotypes of ADRB1 and ADRB2 for each race/ethnicity group (http://research.calit2.net/hap/WebServer.htm) (Halperin et al., Bioinformatics. 2004; 20: 1842-1849).

Baseline characteristics data were expressed as mean±standard deviation (SD) or number (%) as appropriate. Continuous variables not normally distributed were expressed as medians and interquartile ranges. Categorical data were reported as frequencies, and differences between groups were compared with Chi-squared test or Fisher's exact test as appropriate.

To minimize unnecessary multiple testing and maximize the predictive power analyses using ADRB1 and ADRB2 haplotypes were performed using a backward selection procedure. When haplotype analyses were significant, individual genotypes were analyzed in an attempt to ascertain specific SNPs most strongly influencing the haplotype association. The association between the ADRB1, ADRB2 haplotypes/genotypes and blood pressure response was assessed using analysis of covariance (ANCOVA) adjusting for baseline blood pressure and other covariates found associated with blood pressure response. Interactions between haplotypes and race were tested.

A cumulative logic model was used to assess the association between the ADRB1 and ADRB2 haplotypes and the number of antihypertensive drugs (ordinal variable) at the visit when sustained blood pressure control was achieved. As a secondary analysis and to provide some an easy interpretation for clinicians, the number of antihypertensives was treated as a binary variable of whether a patient needed three or more drugs to achieve sustained blood pressure control and multivariate logistic regression analysis was performed. For all analyses, p values <0.05 were considered statistically significant. All statistical analysis was performed in SAS version 9.1 (SAS Institute Inc, Cary, N.C.).

### Example 8

### ADRB1 or ADRB2 Genotype should Influence the Antihypertensive Drug Choice, Independent of Blood Pressure Responses

β-adrenergic receptor gene variants may be associated with cardiovascular risk and β-blocker responses in hypertension and heart failure. To evaluate the relationships between haplotype variation in the β₁- and β₂-adrenergic receptor genes (ADRB1 and ADRB2), incident cardiovascular events, and the outcome of antihypertensive treatment with β-blockers, a substudy cohort of 5895 patients from a randomized, blinded-endpoint trial of antihypertensive therapy in stable coronary artery disease was undertaken. Patients were randomly assigned to treatment with atenolol or sustained-release verapamil. Hydrochlorothiazide and/or trandolapril were added as needed to control blood pressure. Patients were genotyped for 145A>G and 1165C>G in ADRB1, and 46G>A, 79C>G, and 523C>A in ADRB2. The primary outcome was the first occurrence of nonfatal stroke, nonfatal myocardial infarction, or all-cause death. The primary outcome occurred in 256 patients (4.4%) during follow-up (mean 2.8 years). Primary outcome rates were higher in carriers of the ADRB1 145A-1165C haplotype due to higher rates of death (hazard ratio [HR] 3.62, p=0.001). Atenolol tended to reduce the risk of death relative to verapamil among ADRB1145A-1165C carriers (HR 0.64, p=0.04), but not in non-carriers (HR 1.35, p=0.7). Primary outcome rates did not differ across ADRB2 haplotypes in the overall population. Due to a beneficial effect on myocardial infarction and death, atenolol decreased primary outcome risk relative to verapamil in patients without the ADRB2 46G-79G-523C haplotype (HR 0.59, p=0.007), while treatment outcomes were similar in carriers of the 46G-79G-523C haplotype (HR 1.10, p=0.6). Based on this analysis, haplotype variation in ADRB1 was associated with mortality in patients with hypertension and coronary artery disease. β-blockers likely reduced cardiovascular risk to a greater extent than calcium channel blockers in subgroups of patients defined by ADRB1 or ADRB2 polymorphisms.

### Study Population and Baseline Characteristics

The INVEST-GENES was an ethnically diverse elderly population, including a large proportion of women and diabetics. Demographic and clinical characteristics did not differ significantly by treatment strategy (Table 13).

TABLE 13
Baseline characteristics and blood pressure at 24 months

| | Atenolol | | Verapamil SR | |
|---|---|---|---|---|
| | Strategy | | Strategy | |
| | (n = 2973) | | (n = 2922) | |
| Demographic | | | | |
| Age - mean (SD), years | 66 | (9.7) | 66 | (9.6) |
| Age > 70 | 1010 | (34.0) | 971 | (33.4) |
| Female, No. (%) | 1660 | (55.8) | 1637 | (56.0) |
| Race/ethnicity, No. (%) | 1188 | (40.0) | 1221 | (41.8) |
| White | | | | |
| Hispanic | 1438 | (48.4) | 1363 | (46.7) |
| Black | 347 | (11.7) | 338 | (11.6) |
| BMI - mean (SD), kg/m² | 29.5 | (5.6) | 29.3 | (5.6) |
| Medical History, No. (%) | | | | |
| History of MI | 673 | (22.6) | 689 | (23.6) |
| Heart failure (class I-III) | 106 | (3.6) | 95 | (3.3) |
| Stable angina | 2219 | (74.6) | 2168 | (74.2) |
| Unstable angina | 277 | (9.3) | 293 | (10.1) |
| Dyslipidemia | 1779 | (59.8) | 1743 | (59.7) |
| LVH | 463 | (15.6) | 423 | (14.5) |
| Arrhythmia | 191 | (6.4) | 211 | (7.2) |
| Stroke or TIA* | 183 | (6.2) | 228 | (7.8) |
| PVD | 327 | (11.0) | 328 | (11.2) |
| Renal insufficiency | 38 | (1.3) | 54 | (1.9) |
| Diabetes | 851 | (28.6) | 806 | (27.6) |
| Obese | 1226 | (41.2) | 1158 | (39.6) |
| Cancer | 108 | (3.6) | 131 | (4.5) |
| Ever-smoker | 1220 | (41.0) | 1213 | (41.5) |
| Medications, No. (%) | | | | |
| Aspirin/antiplatelet | 1355 | (45.6) | 1339 | (45.8) |
| Other NSAIDs | 718 | (24.2) | 682 | (23.3) |
| Antidiabetic | 736 | (24.7) | 661 | (22.6) |
| Lipid-lowering | 1049 | (35.3) | 1072 | (36.7) |
| Nitrates | 848 | (28.5) | 812 | (27.8) |
| Potassium | 172 | (5.8) | 175 | (6.0) |
| HRT | 393 | (13.2) | 366 | (12.5) |

Abbreviations:
BMI, body mass index;
LVH, left ventricular hypertrophy;
TIA, transient ischemic attack;
PVD, peripheral vascular disease;
NSAIDs, nonsteroidal anti-inflammatory drugs;
HRT, hormone replacement therapy
*p < 0.05 for verapamil SR vs. atenolol

Haplotypes were inferred for 5895 patients who were successfully genotyped at both ADRB1 loci (n=5817) or 2 of 3 ADBR2 loci (n=5877). The genotype and haplotype distributions differed significantly by race/ethnicity (Table 3). Other differences in baseline characteristics by genotype (p<0.05) were as follows: ABDR1145A>G was associated with a higher prevalence of stable angina in whites; ADRB1 1165C>G was associated with a higher prevalence of cancer in blacks and peripheral vascular disease in Hispanics; ADRB2 46G>A was associated with a higher prevalence of diabetes in Hispanics; ADRB2 79C>G lower prevalence of angina in whites and dyslipidemia in blacks; ADRB2 523C>A was associated with a higher prevalence of diabetes in blacks and arrhythmia in Hispanics. Low to moderate levels of LD were noted between the SNPs in ADRB1 (r² 0.04-0.19) and ADRB2 (r² 0.07-0.45).
ADRB1Associations with Primary and Secondary Outcomes

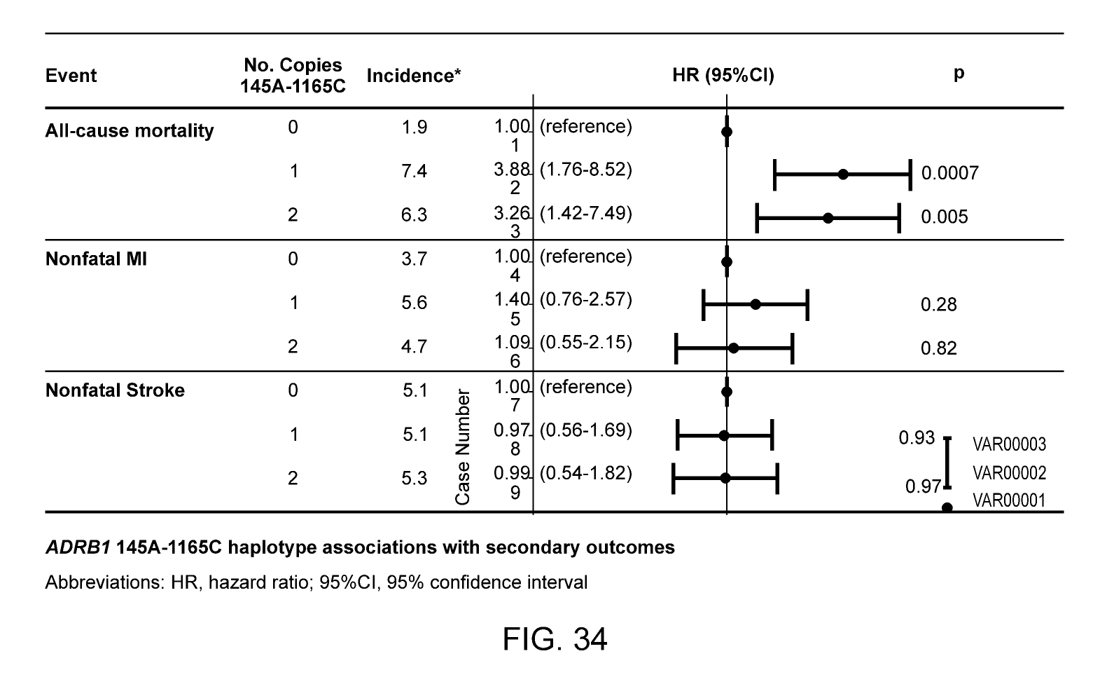

The 2 variant loci in ADRB1 formed 3 common haplotypes (Table 15). Systolic and diastolic blood pressures did not differ according to ADRB1 haplotype. The model based on the 145A-1165C haplotype best characterized the risk for the primary outcome (log-rank p=0.02). Patients with 1 or 2 copies of the 145A-1165C had a relatively higher risk for the primary outcome relative to non-carriers (145A-1165 carriers vs. non-carriers HR 1.51, 95% CI 1.07-2.12, p=0.02; Table 15). The 145A-1165C haplotype was significantly associated with mortality (145A-1165C carriers vs. non-carriers HR 3.66, 95% CI 1.68-7.99, p=0.001), while no association was noted for nonfatal MI or nonfatal stroke (FIG. 34). The increased risk of death among carriers of the 145A-1165C haplotype was consistent across racial/ethnic groups (145A-1165C carriers vs. non-carriers: white HR 2.42, 95% CI 0.88-6.70, p=0.09; Hispanic HR 3.02, 95% CI 0.95-10.28, p=0.06; black HR not calculated because no events occurred in non-carriers) and persisted after further adjustment for ancestry (145A-1165C carriers vs. non-carriers: white HR 2.32, 95% CI 0.84-6.44, p=0.1; Hispanic HR 2.95, 95% CI 0.89-9.78, p=0.08; black HR not calculated). Given the reduced power when evaluating race groups separately, all subsequent analyses are presented for the combined population, controlling for race/ethnicity. In genotype-based analyses, neither ADRB1 SNP was associated with the primary outcome, although homozygotes for the 1165G allele were at lower risk for death (C/G vs. C/C HR 0.21, 95% CI 0.51-1.18, p=0.23; G/G vs. C/C HR 0.21, 95% CI 0.05-0.84, p=0.03). The 145A>G SNP was not associated with any secondary outcomes (data not shown). These data suggest that ADRB1 haplotype is more informative than single SNP analysis.

ADRB2 Associations with Primary and Secondary Outcomes

The 2 variant loci in ADRB2 formed 3 common haplotypes (Table 14).

TABLE 14
Minor allele and haplotype frequencies by race/ethnicity

| | All | White | Hispanic | Black |
|---|---|---|---|---|
| ADRB1 (%) | n = 5817 | n = 2375 | n = 2766 | n = 676 |
| 145G | 17.8 | 12.2 | 20.8 | 23.2 |
| 1165G | 29.0 | 27.2 | 28.4 | 39.1 |
| 145A-1165C | 53.4 | 60.8 | 51.0 | 37.6 |
| 145A-1165G | 28.9 | 27.0 | 28.3 | 39.2 |
| 145G-1165C | 17.6 | 12.1 | 20.6 | 23.2 |
| ADRB2 (%) | n = 5877 | n = 2400 | n = 2795 | n = 682 |
| 46A | 43.4 | 38.7 | 45.3 | 51.0 |
| 79G | 32.2 | 41.8 | 28.7 | 16.3 |
| 523A | 25.2 | 19.3 | 27.4 | 34.8 |
| 46A-79C-523C | 41.1 | 35.9 | 43.3 | 48.2 |
| 46G-79G-523C | 29.7 | 39.9 | 25.7 | 14.3 |
| 46G-79C-523A | 22.2 | 16.8 | 23.8 | 32.4 |
| Other | 7.0 | 7.4 | 7.2 | 5.1 |

Systolic and diastolic blood pressures did not differ by ADRB2 haplotype. None of the ADRB2 haplotype models revealed differential risk for the primary outcome in the overall population (Table 15) or in any of the racial/ethnic subgroups.

TABLE 15
ADRB1 and ADRB2 haplotype associations with primary outcome

| | | | | | | Expanded | |
|---|---|---|---|---|---|---|---|
| | No. | No. Events/ | | Reduced Model^{†} | | Model | |
| Gene/Haplotype | Copies | Total | Incidence* | HR (95% CI) | p | HR (95% CI) | p |
| ADRB1 | | 255/5817 | 15.6 | | | | |
| 145A-1165C | 0 | 40/1319 | 10.8 | 1.00 | | 1.00 | |
| | 1 | 139/2788 | 17.8 | 1.59 (1.12-2.27) | 0.01 | 1.63 (1.14-2.32) | 0.007 |
| | 2 | 76/1710 | 15.7 | 1.35 (0.91-1.99) | 0.14 | 1.40 (0.95-2.06) | 0.09 |
| 145A-1165G | 0 | 137/2941 | 16.6 | 1.00 | | 1.00 | |
| | 1 | 101/2390 | 15.1 | 0.90 (0.70-1.17) | 0.43 | 0.93 (0.72-1.20) | 0.56 |
| | 2 | 17/486 | 12.3 | 0.73 (0.44-1.21) | 0.22 | 0.79 (0.48-1.31) | 0.36 |
| 145G-1165C | 0 | 175/3955 | 15.7 | 1.00 | | 1.00 | |
| | 1 | 76/1675 | 16.2 | 1.12 (0.86-1.48) | 0.41 | 1.13 (0.86-1.49) | 0.37 |
| | 2 | 4/187 | 7.9 | 0.63 (0.23-1.71) | 0.37 | 0.66 (0.24-1.78) | 0.41 |
| ADRB2 | | 255/5877 | 15.4 | | | | |
| 46A-79C-523C | 0 | 96/2033 | 16.7 | 1.00 | | 1.00 | |
| | 1 | 120/2812 | 15.2 | 0.94 (0.72-1.23) | 0.66 | 0.93 (0.71-1.22) | 0.62 |
| | 2 | 39/1032 | 13.4 | 0.87 (0.60-1.26) | 0.46 | 0.85 (0.58-1.24) | 0.39 |
| 46G-79G-523A | 0 | 166/3589 | 16.3 | 1.00 | | 1.00 | |
| | 1 | 78/1876 | 14.1 | 0.94 (0.72-1.23) | 0.64 | 0.93 (0.71-1.22) | 0.60 |
| | 2 | 11/312 | 12.7 | 0.88 (0.48-1.63) | 0.69 | 0.78 (0.42-1.45) | 0.44 |
| 46G-79G-523C | 0 | 121/2930 | 14.8 | 1.00 | | 1.00 | |
| | 1 | 101/2339 | 15.2 | 0.94 (0.72-1.23) | 0.66 | 0.98 (0.74-1.28) | 0.85 |
| | 2 | 33/608 | 18.9 | 1.07 (0.72-1.60) | 0.72 | 1.15 (0.77-1.71) | 0.49 |

Abbreviations:
HR, hazard ratio;
95% CI, 95% confidence interval
Crude incidence per 1000 patient-years
Adjusted for age, sex, race/ethnicity
Adjusted for age, sex, race/ethnicity and additional covariates selected by stepwise selection procedure

### Pharmacogenetic Associations

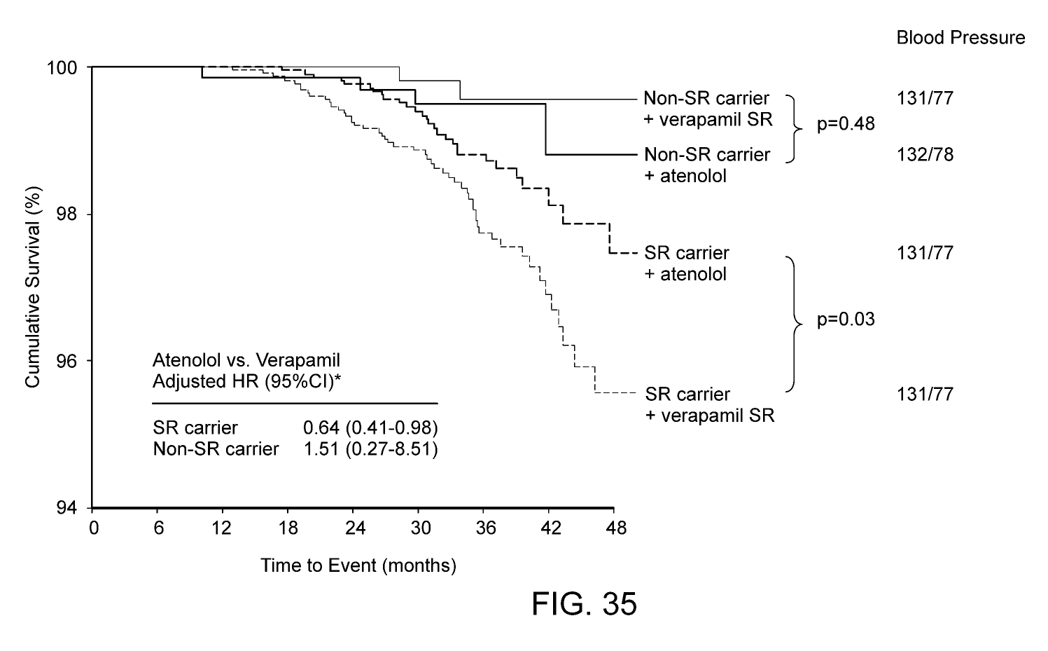

The treatment strategies did not differ in terms of the primary outcome (HR 0.81, 95% CI 0.63-1.05, p=0.12) or blood pressure. The risk for the primary outcome was similar between atenolol- and verapamil SR-treated patients within each 145A-1165C haplotype strata. For all-cause mortality, where the main ADRB1 haplotype effect was most pronounced, patients with 1 or 2 copies of the 145A-1 165C haplotype demonstrated significantly lower rates mortality rates with atenolol as compared to verapamil SR (FIG. 35). Alternatively, when evaluating haplotype risks within drug treatment groups, the risk for death associated with the 145A-165C haplotype was diminished in β-blocker treated patients, yet clearly prominent among those receiving verapamil SR, with and without adjustment for clinical covariates (FIG. 35). Blood pressures across treatment and haplotype groups were similar at the end of follow-up (FIG. 35).

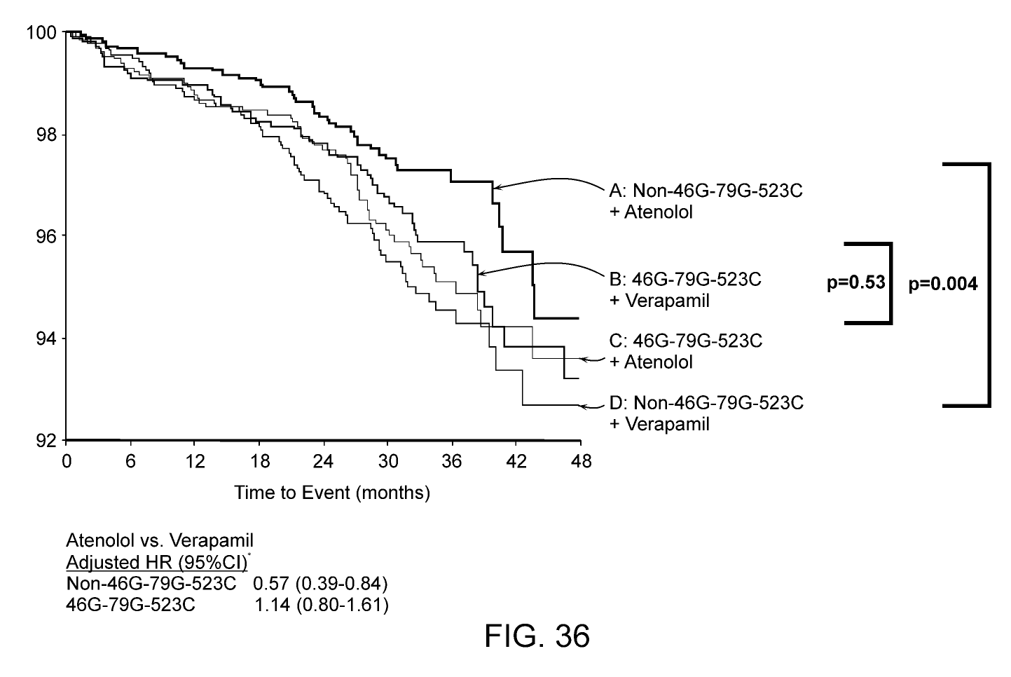

Pharmacogenetic analysis of common ADRB2 haplotypes also revealed a differential risk for the primary outcome between atenolol and verapamil SR across the 46G-79G-523C haplotype model (FIG. 36). Non-carriers of the haplotype were at lower risk for the primary outcome if treated with atenolol, whereas atenolol and verapamil SR did not differ in patients with 1 or 2 copies of the 46G-79G-523C haplotype (atenolol vs. verapamil SR: non-carriers HR 0.59, 95% CI 0.41-0.87, p=0.007; carriers HR 1.10 95% CI 0.78-1.56, p=0.58). The difference in drug-related outcomes among non-carriers of the 46G-79G-523C haplotype was driven by mortality and to a lesser extent, nonfatal MI (FIG. 36). Genotype-based analysis revealed that this was driven largely by the 523C>A genotype (P_{interaction}=0.02), but similar trends that were consistent with the haplotype association were also noted for the 46G>A and 79C>G SNPs, again supporting that the haplotype analysis was a more powerful. Again, blood pressure did not vary as a function of randomized drug therapy or ADRB2 haplotype, suggesting such differences are not predictable genetic associations with BP response (Table 16).

TABLE 16
Primary and secondary outcomes by ADRB2 46G-79G-523C haplotype and
antihypertensive drug therapy

| | No. Copies | Incidence* | | HR (95% CI)^{‡} | |
|---|---|---|---|---|---|
| Haplotype | 46G-79G-523C | Atenolol^{†} | Verapamil | Atenolol vs. Verapamil | p |
| Primary Outcome | 0 | 10.8 | 18.3 | 0.60 (0.41-0.87) | 0.007 |
| | 1 | 14.9 | 14.5 | 1.03 (0.69-1.54) | 0.89 |
| | 2 | 22.3 | 17.0 | 1.35 (0.68-2.67) | 0.39 |
| Pinteraction | | | | 0.05 | |
| All-cause Mortality | 0 | 3.2 | 7.3 | 0.45 (0.23-0.86) | 0.02 |
| | 1 | 5.7 | 7.0 | 0.81 (0.44-1.50) | 0.51 |
| | 2 | 7.7 | 4.2 | 1.80 (0.51-6.39) | 0.36 |
| Pinteraction | | | | 0.11 | |
| Nonfatal MI | 0 | 3.5 | 5.9 | 0.60 (0.31-1.16) | 0.13 |
| | 1 | 5.8 | 2.9 | 1.94 (0.89-4.20) | 0.09 |
| | 2 | 9.1 | 6.3 | 1.53 (0.51-4.57) | 0.45 |
| Pinteraction | | | | 0.06 | |
| Nonfatal Stroke | 0 | 4.2 | 5.1 | 0.85 (0.44-1.62) | 0.62 |
| | 1 | 4.8 | 4.7 | 1.01 (0.50-2.05) | 0.97 |
| | 2 | 5.1 | 6.3 | 0.79 (0.22-2.81) | 0.72 |
| Pinteraction | | | | 0.94 | |

Abbreviations:
HR, hazard ratio;
95% CI, 95% confidence interval
*Crude incidence per 1000 patient-years
^{†}Includes only patients ever-exposed to atenolol (90.8% in atenolol strategy)
^{‡}Hazard ratios based on reduced model adjusting for age, sex, race/ethnicity

Identifying genetic markers for cardiovascular risk is likely to improve cardiovascular risk stratification and identify those requiring more aggressive management of hypertension and related chronic diseases. Common SNPs in the genes encoding the β₁- and β₂-adrenergic receptors alter receptor activity and have physiological consequences. Consistent with the known functionality of the β₁-adrenergic receptor variants, an association was identified between ADRB1 haplotypes and the risk of death. More importantly, these data suggested that β-blockers offset this mortality risk, consistent with the observation that patients bearing the wild-type alleles are uniquely responsive to β-blocker therapy. ADBR2 variants were similarly associated with treatment outcomes. The pharmacogenetic evidence for β-blockers and adrenergic receptor genes was highly convincing, particularly for ADRB1, and these data suggested that a patient's genotype should influence the antihypertensive drug choice independent of blood pressure responses.

The results described in Example 8 were carried out using the following methods and materials.

### INVEST-GENES Design and Participants

The INVEST was a prospective, randomized, open-label, blinded-endpoint (PROBE) trial designed to compare antihypertensive treatment outcomes in 22576 patients. The INVEST-GENES cohort consisted of 5979 patients from 184 sites in the United States and Puerto Rico who provided DNA samples and additional written informed consent for genetic studies. The details of the INVEST methods and main outcomes were previously reported (Pepine et al., Jama. Dec. 3, 2003; 290(21):2805-2816). Briefly, the INVEST included hypertensive patients over the age of 50 with stable CAD, as defined by previous myocardial infarction, angiographic evidence of stenosis in at least one major coronary artery, myocardial ischemia detected by two methods, or stable angina. Patients were randomly assigned to receive either verapamil SR or atenolol. Trandolapril was recommended for all patients with heart failure, renal dysfunction, or diabetes. Hydrochlorothiazide and/or trandolopril were added as needed to achieve JNC VI blood pressure targets. Patients were followed every 6 weeks for the first 6 months, and every 6 months thereafter until 2 years after the last patient was enrolled. The extent of blood pressure control and cardiovascular outcomes were similar between the treatment strategies (Pepine et al., Jama. Dec. 3, 2003; 290(21):2805-2816)

### Outcomes

The primary outcome was a composite of the first occurrence of all-cause mortality, nonfatal myocardial infarction, and nonfatal stroke. Secondary outcomes included the individual components of the primary outcome. Events were adjudicated by a committee that was blinded to treatment strategy.

### Genotyping

Buccal tissue samples were obtained by mouthwash and genomic DNA was isolated using Gentra Systems PureGene kit. Patients were genotyped for 2 variants in ADRB1 (145A>G, rsl801252; 1165C>G, rsl801253) and 3 variants in ADRB2 (46G>A, rs1042713; 79A>G, rs1042714; 523C>A, rs1042718) using pyrosequencing (Biotage, Uppsala, Sweden) and TaqMan allelic discrimination (Applied Biosystems, Foster City, Calif.). Genotype accuracy was verified by genotyping 5% duplicate samples for each SNP on the alternate platform.

Ancestry informative markers (AIMs; n=87) were genotyped using either allele-specific PCR with universal energy transfer labeled primers or competitive allele-specific PCR at Prevention Genetics (Marshfield, Wis.).

### Statistics

Hardy-Weinberg equilibrium was tested for each racial/ethnic group using X² analysis. Haplotypes were reconstructed separately for each racial/ethnic group from raw genotype data using PHASE software (version 2.1) (Pritchard et al., Am J Hum Genet. July 2000; 67(1):170-181) for patients that were successfully genotyped at both ADRB1 loci and at least 2 ADRB2 loci. Each haplotype was coded based on the number of copies (0, 1, or 2). Linkage disequilibrium (LD; r²) between the SNPs in each racial/ethnic group was estimated using Haploview (Barrett et al., Bioinformatics. Jan. 15, 2005; 21(2):263-265). Demographic and baseline clinical characteristics were compared by haplotype and genotype using X² tests for categorical data, and t-tests, ANOVA, or a nonparametric equivalent for continuous data. Repeated measures analysis of variance was used to evaluate differences in blood pressure between treatment strategies and haplotypes. Main effects of each haplotype on the primary outcome were analyzed using Kaplan-Meier analysis with pooled log-rank tests adjusted across racial/ethnic strata Cox proportional hazards regression was performed to estimate hazard ratios (HR) and 95% confidence intervals (95% CI) for each copy of the haplotype or variant allele relative to absence of the haplotype or variant allele. The regression model initially adjusted for race/ethnicity, age, sex, and treatment strategy (reduced model). The following covariates were subsequently entered into the model using the stepwise procedure if p<0.1 and retained if p<0.05 (expanded model): history of heart failure, MI, diabetes, stroke or transient ischemic attack, renal insufficiency, dyslipidemia, left ventricular hypertrophy, peripheral vascular disease, stable angina, unstable angina, arrhythmia, cancer, ever-smoking, body mass index, and baseline systolic and diastolic blood pressures. Significant haplotype associations with the primary outcome were followed by analysis of the secondary endpoints, as well as genotype-based analysis. The threshold for significance was set at α=0.05. All statistical analyses were performed using SAS version 9.1 (SAS Institute, Cary, N.C.).

All common haplotypes were tested for pharmacogenetic associations with the primary outcome and for the secondary outcomes where significant main associations or pharmacogenetic associations were observed. Pharmacogenetic analyses focused on the randomized study drugs, verapamil SR and atenolol, the use of which was mutually exclusive. Atenolol and verapamil SR were started at baseline in 90.8% and 100% patients in the respective strategy, and as such, only exposed patients were included in the analysis, being classified as either ever-exposed or never-exposed to the primary study drugs. Pharmacogenetic associations were evaluated by Kaplan-Meier analysis with pairwise log-rank tests, and by testing interaction terms in the Cox proportional hazards regression models. In addition to adjusting for selected demographic and clinical covariates, pharmacogenetic analyses also adjusted for trandolapril and HCTZ exposures, which were modeled as time-varying covariates, using the average dose level prior to event or censoring to define exposure, due to the differences in time to initiation and overlapping use. The relative risk differences between atenolol and verapamil SR were estimated in haplotype stratified analyses. Similarly, haplotype associations were estimated in analyses stratified by the randomized study drugs.

To investigate the potential influence of population stratification, relative proportion of European, Native American, and West African ancestry proportions were determined based on the 87 AIMs using the Bayesian clustering algorithm implemented in STRUCTURE (version 2.0) (Pritchard et al., Am J Hum Genet. July 2000; 67(1):170-181). Separate regression models that adjusted for the proportion of West African and Native American ancestry were constructed for those patients with genotype data for more than 50% of the AIMs. All analyses were performed with and without stratification by race/ethnicity, but are presented for the combined population unless the results differed across racial/ethnic strata in the interest of maintaining power.

### Example 9

### Beta Blocker Therapy Had a Protective Effect in Subjects with rs10848683 and in Subjects with RS1051375

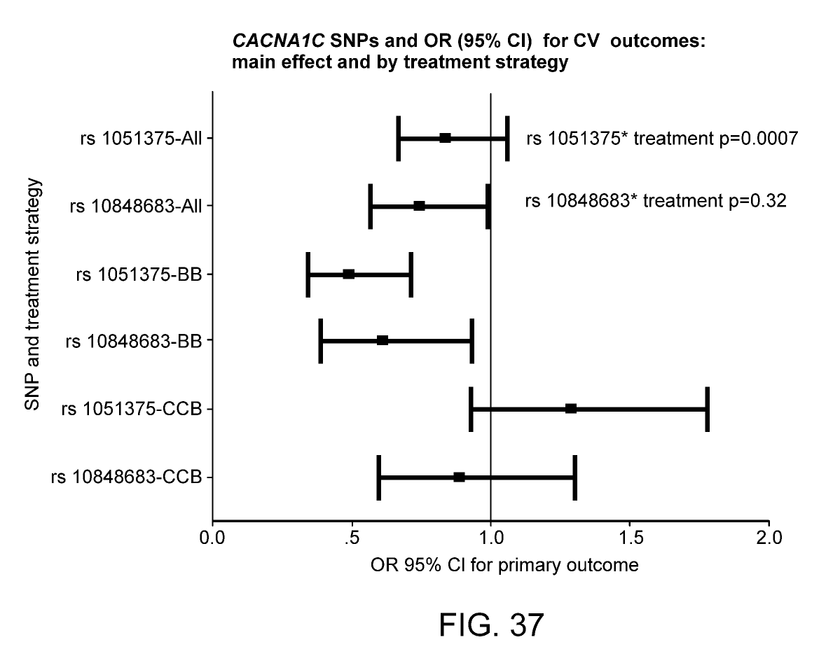

CACNA1C encodes the L-type calcium channel, which is the protein target for all marketed calcium channel blockers (CCBs). There was minimal linkage disequilibrium in CACNA1C and CACNB2. 8 SNPs in CACNA1C were examined in the case-control group. Two SNPs, NCBI reference assembly sequence: rs1051375 (Chromosome 12, position 2659140 (+); nucleic acid sequence AGCACA/GGTCAG) and NCBI reference assembly sequence: rs10848683 (Chromosome 12, position 2661391 (+); nucleic acid sequence: CGTTCC/TGA/GTGT) were associated with significantly better outcomes with β-blocker therapy (FIG. 37). There were no differences in outcomes with CCB therapy, and in one case (rs1051375), there was a highly significant gene*treatment interaction (p<0.0007). When considered separately by race, findings were similar. Findings by event type are shown in Table 17 for rs1051375 and suggest a general consistency of effect across event types, with data indicated increased risk with CCB therapy and protection with β-blockers (BB) among variant carriers for this SNP.

TABLE 17
CACNA1C rs1051375 * outcomes pharmacogenetics effects (i.e.
drug x gene interaction)

| | SNP effect in | SNP effect in | RR: BB vs | SNP × |
|---|---|---|---|---|
| Event | CCB arm | BB arm | CCB by | treatment p |
| type | (OR, 95% CI) | (OR, 95% CI) | genotype | value |
| Death | 1.28 (0.82, 2.0) | 0.64 (0.37-1.12) | 0.50 | 0.0198 |
| MI | 0.97 (0.61-1.53) | 0.38 (0.22-0.68) | 0.39 | 0.019 |
| Stroke | 1.83 (1.13-2.97) | 0.69 (0.40, 1.17) | 0.38 | 0.098 |

These data also indicated that there is a significantly increased risk for CACNA1C variant carriers for stroke with CCB therapy, and that there is significant protection for CACNA1C variant carrier for myocardial infarction with beta blocker therapy (FIG. 37). Table 18 shows data for rs10848683.

TABLE 18
CACNA1C rs10848683 * outcomes
pharmacogenetics effects

| Event | SNP effect in CCB arm | SNP effect in BB arm |
|---|---|---|
| type | (OR, 95% CI) | (OR, 95% CI) |
| Death | 0.659 (0.365, 1.192) | 0.661 (0.345, 1.266) |
| MI | 0.733 (0.415, 1.294) | 0.451 (0.232, 0.878) |
| Stroke | 1.340 (0.767, 2.341) | 0.706 (0.375, 1.329) |

Table 18 suggests that there is significant protection associated with beta-blocker therapy for variant CA CNA1C carriers.

### Example 10

### Treatment with Beta-Blocker Reduced Cardiovascular Risk Associated with CACNB2 SNP rs120036

In CACNB2, the presence of a single nucleotide polymorphism, rs2357928 was suggestive of a striking pharmacogenetic effect, and an effect on outcomes independent of drug therapy Specifically, the rs2357928 variant was protective for reducing risk of adverse cardiovascular events (OR: 0.70 [0.51-0.96], p=0.028). When considered by treatment strategy, there was a significant drug x genotype interaction (p=0.017). Specifically, among CCB-treated patients there was no effect by rs2357928 genotype on the primary outcome (OR: 1.00 [0.64-1.57], p=1.0), but there was a protective effect among β-blocker-treated patients (OR: 0.50 [0.31-0.79], p=0.003). This observation was consistent across racial groups. The data for the individual components of the composite outcome are shown in Table 19.

TABLE 19
CACNA1C rs120036 * outcomes
pharmacogenetics effects

| Event | SNP effect in CCB arm | SNP effect in BB arm |
|---|---|---|
| type | (OR, 95% CI) | (OR, 95% CI) |
| Death | 0.95 (0.51-1.78) | 1.08 (0.51-2.3) |
| MI | 0.91 (0.44-1.90) | 0.56 (0.27-1.12) |
| Stroke | 1.11 (0.54-2.24) | 0.38 (0.19-0.73) |

These data show that there is significant protection among rs2357928 variant carriers for the risk of stroke if treated with a beta-blocker. The data indicates protection against myocardial infarction among variant carriers treated with beta-blockers.

### Example 11

### Treatment with Beta-Blocker Reduced Risk Associated with ALOX5 Polymorphism

There is a tandem repeat promoter polymorphism in an Sp1 motif ALOX5 that has not yet been assigned an NCBI SNP accession (“rs”) number. The polymorphism location is shown below by underlining

### Goldenpath Position: Chr10:45,189,558-45,189,587 (for Wild-Type Allele):

GACACCTCGCTGAGGAGAGACCCAGGAGCGAGGCCCCTGCCCCGCCCGAG
GCGAGGTCCC
GCCCAGTCGGCGCCGCGTGAAGAGTGGGAGAGAAGTACTGCGGGGGCGGG
GGCGGGGGCG
GGGGCGGGGGCGGGGGCAGCCGGGAGCCTGGAGCCAGACCGGGGCGGGGC
CGGGACCGGG
GCCAGGGACCAGTGGTGGGAGGAGGCTGCGGCGCTAGATGCGGACACCTG
GACCGCCGCG
CCGAGGCTCCCGGCGCTCGCTGCTCCCGCGGCCCGCGCCATGCCCTCCTA
CACGGTCACC

As shown in Table 20 below, the ALOX5 polymorphism was strongly associated with risk for adverse cardiovascular outcomes. Treatment with a beta-blocker reduced the risk associated with this polymorphism. The effect (odds ratio) was slighly increased in those who received verapamil therapy.

TABLE 20

| | | | Chi Square- | |
|---|---|---|---|---|
| | OR | CI | Type 3 | p-value |
| Variant Carriers | | | | |
| Overall | 1.782 | 1.291-2.459 | 12.3511 | 0.0004 |
| Drug Interaction | | | 0.0309 | 0.8605 |
| By Drug Therapy | | | | |
| atenolol | 1.577 | 0.975-2.553 | | |
| verapamil | 1.964 | 1.261-3.057 | | |

### Example 12

### Atenolol Therapy is Protective for Diabetes in ADRB1 Gly389 Homozygotes

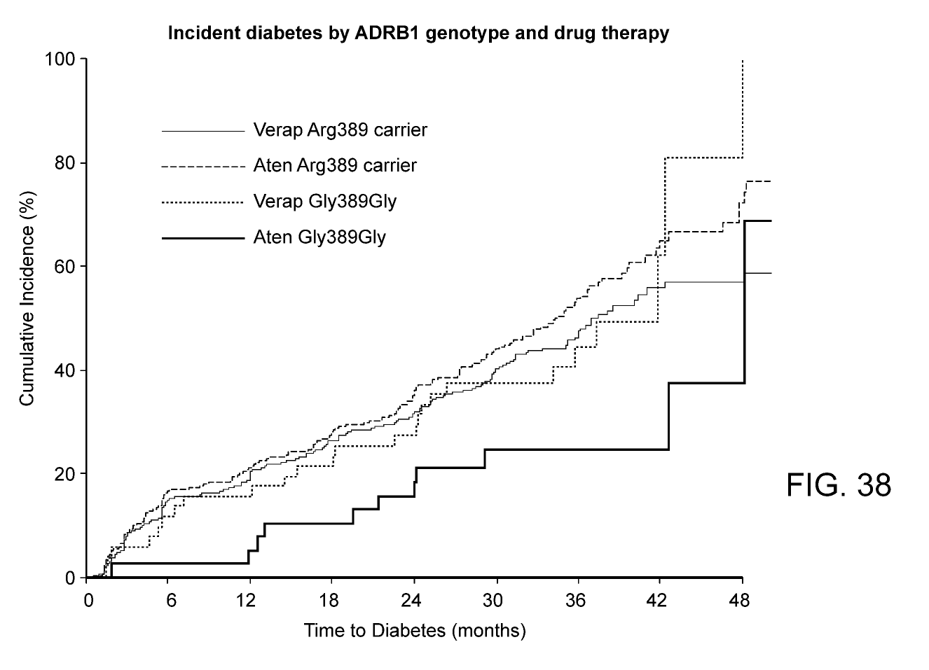

ADRB1 codon 389 genotype influences the risk for development of diabetes during antihypertensive therapy (FIG. 38) Specifically, among Arg389 carriers there was a trend toward increased risk for development of diabetes during treatment with atenolol versus verapamil (HR 1.20, 95% CI: 0.98-1.46). However, among ADRB1Gly389 homozygotes, atenolol therapy appeared to be protective compared to verapamil therapy, (HR: 0.43, 95% CI: 0.21-0.86). This represented a significant gene-drug interaction (p=0.016), with all analyses adjusted for race/ethnicity. Considered a different way, the risk of developing diabetes was significantly different by genotype among atenolol-treated patients (p=0.02), but was not different by genotype among verapamil-treated patients (p=0.29). Thus, identification of a hypertensive subject as homozygous for ADRB1Gly389 indicates that the subject's hypertension should be treated with atenolol rather than verapamil.

Published single nucleotide polymorphisms described herein are available in the NCBI SNP database, which is incorporated herein in its entirety. Table 21 provides a list of SNPs that are useful for assessing cardiovascular risk and treatment selection.

TABLE 21

| | | Chromosomal | |
|---|---|---|---|
| | SNP Acc. No. | Pos'n | |
| 1 | rs7908738 | 18467752 | |
| 2 | rs7900001 | 18467854 | |
| 3 | rs7900124 | 18467913 | |
| 4 | rs34418804 | 18467976 | |
| 5 | rs7909119 | 18467990 | |
| 6 | rs10764319 | 18468421 | genotyped |
| 7 | rs12764271 | 18469413 | |
| 8 | rs12572321 | 18470226 | |
| 9 | rs7069292 | 18586994 | |
| 10 | rs2482107 | 18587337 | |
| 11 | rs2489221 | 18587384 | |
| 12 | rs10828387 | 18587789 | genotyped |
| 13 | rs10828388 | 18588488 | |
| 14 | rs11013218 | 18588495 | |
| 15 | rs7099380 | 18589022 | |
| 16 | rs1106380 | 18589121 | genotyped |
| 17 | rs2357928 | 18589647 | |
| 18 | rs10828542 | 18667291 | |
| 19 | rs12259560 | 18667826 | |
| 20 | rs10828545 | 18669609 | |
| 21 | rs6482385 | 18728889 | genotyped |
| 22 | rs3829133 | 18730674 | |
| 23 | rs16917273 | 18730705 | genotyped |
| 24 | rs3750592 | 18731024 | genotyped |
| 25 | rs4485000 | 18829730 | genotyped |
| 26 | rs12357063 | 18843427 | genotyped |
| 27 | rs4314963 | 18856639 | genotyped |
| 28 | rs2228645 | 18868377 | genotyped |
| 29 | rs7079776 | 18870675 | |
| 30 | rs12267405 | 18870907 | |

The sequence of particular SNPs is provided below:

rs1042718
TCTTGCCCATTCAGATGCACTGGTAC[A/C]GGGCCACCCACCAGGAAGC
CATCAA
rs2656842
GGGAGGGCAGGTGGAGAAGGCATTGT[G/T]CTGCAAGTGGGGAGCAGCC
CTGGGG
rs2656841
TGGGAGGGCAGGTGGAGAAGGCATTG[G/T]GCTGCAAGTGGGGAGCAGC
CCTGGG
rs2247570
GAtaagtgctttaaatgcattcttgt[A/G]tctaatccttacagtaacc
ctgctg
rs1978331
rs1978331 [Homo sapiens]
TGGTATAACACTGTGTTGATCCACAC[C/T]GTTCACTGTTCTATGTAAG
GTAGTT
rs1051375
CCTCGCCCCGCCGGCTACCCCAGCAC[A/G]GTCAGCACTGTGGAGGGCC
ACGGGC
rs10848683
CTGTCCCAGCAGGGAAAGGCACGTTC[C/T]GGTGTGTGAGGATCTGGAG
CTCAGG
rs1801252
CCTCGTTGCTGCCTCCCGCCAGCGAA[A/G]GCCCCGAGCCGCTGTCTCA
GCAGTG
rs1801253
GCCCCGACTTCCGCAAGGCCTTCCAG[C/G]GACTGCTCTGCTGCGCGCG
CAGGGC
rs2247570
GAtaagtgctttaaatgcattcttgt[A/G]tctaatccttacagtaacc
ctgctg
rs1978331
TGGTATAACACTGTGTTGATCCACAC[C/T]GTTCACTGTTCTATGTAAG
GTAGTT
rs2660845
ACTTCATAGTGTCTACCACTGGCCCC[A/G]CGGGGCTCTGCAGCTTCCA
CTTGAG
rs1051375
CCTCGCCCCGCCGGCTACCCCAGCAC[A/G]GTCAGCACTGTGGAGGGCC
ACGGGC
rs10848683
CTGTCCCAGCAGGGAAAGGCACGTTC[C/T]GGTGTGTGAGGATCTGGAG
CTCAGG

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of identifying a subject as having an increased or decreased propensity to have an adverse cardiovascular event comprising analyzing a subject sample for an alteration in the nucleic acid sequence of at least one gene selected from the group consisting of α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), arachidonate 5-lipoxygenase-activating protein (ALOX5AP), CACNA1C, CACNB2, and ALOX5 gene relative to a wild-type reference sequence, the presence or absence of the alteration indicating propensity to have an adverse cardiovascular event.

2. (canceled)

3. The method of claim 2, wherein the adverse cardiovascular event is a fatal or nonfatal myocardial infarction or a stroke.

4. The method of claim 2, where the alteration in the nucleic acid sequence is selected from the group consisting of:
in the ADD1 gene the ADD1 460Trp polymorphism;
in the β1-adrenergic receptor gene the 46G-79C-523C haplotype, the 49S-389R haplotype, the 49S-389R haplotype, 46G-79G-523C haplotype;
in the LTA4H gene the rs2247570 TT or rs1978331 AA variant;
in the KCNMB1 gene the Lys65 variant or the Leu110 variant; and
in the ALOX5AP gene the GTC haplotype.

5-11. (canceled)

12. The method of claim 1, wherein the method further comprises determining one or more factors selected from the group consisting of the subject's age, race or ethnic group, sex, body mass index, blood pressure, smoking status, low density lipid (LDL) or high density lipid (HDL) cholesterol level, systolic blood pressure, dyastolic blood pressure, history of heart failure, diabetes, renal insufficiency, or left ventricular hypertrophy.

13. A method for identifying a subject as having an allele variant correlated with increased or decreased efficacy of a treatment, said method comprising analyzing a nucleic acid sample obtained from said subject to determine whether said sample comprises at least one allele variant in a gene selected from the group consisting of α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), arachidonate 5-lipoxygenase-activating protein (ALOX5AP), CACNA1C, CACNB2, and ALOX5 gene relative to a wild-type reference sequence, said variant being correlated to increased or decreased responsiveness to a treatment for a cardiovascular disorder.

14. The method of claim 13, wherein the treatment is β1-blocker therapy or verapamil SR therapy.

15. (canceled)

16. The method of claim 13, wherein the allele variant Lys65 in the calcium activated potassium channel (KCNMB1) gene or the allele variants 46G-79C-523C in the β2-adrenergic receptor identifies the subject as responsive to verapamil therapy.

17. The method of claim 13, wherein the allele variant Leu110 in the calcium activated potassium channel (KCNMB1) gene identifies the subject as responsive to a calcium channel blocker.

18. (canceled)

19. The method of claim 13, wherein the absence of the allele variants 46G-79C-523C in the β2-adrenergic receptor gene or the homozygous allele variants AA in the LTA4H gene identifies responsiveness to atenolol therapy.

20. The method of claim 13, wherein the allele variants 49S-389R in the β1-adrenergic receptor gene identifies responsiveness to β-blocker therapy.

21. (canceled)

22. The method of claim 13, wherein the G-allele variant in the LTA4H gene identifies equal responsiveness to verapamil SR or atenolol therapy.

23. The method of claim 13, wherein the method further comprises determining one or more factors selected from the group consisting of the subject's age, race, sex, body mass index, blood pressure, smoking status, low density lipid (LDL) or high density lipid (HDL) cholesterol level, systolic blood pressure, dyastolic blood pressure, history of heart failure, diabetes, renal insufficiency, or left ventricular hypertrophy.

24. The method of claim 13, wherein the method identifies an allelic variant selected from the group consisting of:
a CACNB1 single nucleotide polymorphism (SNP) that is NCBI reference assembly sequence rs1051375 or rs10848683,
CACNB2 single nucleotide polymorphism identified by NCBI Ref Seq: rs120036,
an allelic variant in ALOX5, and
an allelic variant at Gly 389 of ADRB1.

25. A method for identifying a cardiovascular therapy for a subject, said method comprising:
(a) analyzing a nucleic acid sample obtained from said subject to determine whether the sample comprises at least one allele variant of a gene selected from the group consisting of α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), CACNA1C, CACNB2, and ALOX5 and arachidonate 5-lipoxygenase-activating protein (ALOX5AP), wherein the variant is correlated to increased or decreased responsiveness to a treatment for a cardiovascular condition; and
(b) identifying a treatment regimen for the subject having said allele.

26-40. (canceled)

41. The method of claim 40, wherein the biological fluid sample is saliva, urine, or blood.

42-51. (canceled)

52. A nucleic acid microarray for use in the method of claim 1, comprising a nucleic acid molecule comprising at least a fragment of two or more genes selected from the group consisting of CACNA1C, CACNB2, ALOX5, α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), and arachidonate 5-lipoxygenase-activating protein (ALOX5AP) gene.

53. (canceled)

54. A primer set for use in the method of claim 1, comprising primers that bind to two or more genes selected from the group consisting of CACNA1C, CACNB2, ALOX5, α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), and arachidonate 5-lipoxygenase-activating protein (ALOX5AP) gene.

55-58. (canceled)

59. A kit for use in the method of claim 1, the kit comprising a nucleic acid sequence that hybridizes to a gene selected from the group consisting of CACNA1C, CACNB2, ALOX5, α-adducin (ADD1) gene, calcium activated potassium channel (KCNMB1) gene, β1-adrenergic receptor (ADRB1) gene, β2-adrenergic receptor (ADRB2) gene, leukotriene A4 hydrolase (LTA4H), and arachidonate 5-lipoxygenase-activating protein (ALOX5AP) gene.

60-73. (canceled)
